(19) United States
(12) Patent Application Publication
(10) Pub. No.: US 2020/0276318 A1
(43) Pub. Date: Sep. 3, 2020

(54) ENGINEERED CELLS AND AGENT COMPOSITIONS FOR THERAPEUTIC AGENT DELIVERY AND TREATMENTS USING SAME

(71) Applicants: UNIVERSITY OF WASHINGTON, Seattle, WA (US); SEATTLE CHILDREN'S RESEARCH INSTITUTE, Seattle, WA (US); COMMONWEALTH SCIENTIFIC AND INDUSTRIAL RESEARCH ORGANISATION, Acton, Australian Capitol Territory (AU)

(72) Inventors: STAYTON S. Patrick, Seattle, WA (US); Katherine MONTGOMERY, Seattle, WA (US); Courtney CRANE, Seattle, WA (US); Michael JENSEN, Seattle, WA (US); John CHIEFARI, Acton (AU); James MACDONALD, Acton (AU); Maarten DANIAL, Acton (AU); Fei HUANG, Acton (AU); Almar POSTMA, Acton (AU); Kathleen TURNER, Acton (AU)

(73) Assignees: UNIVERSITY OF WASHINGTON, Seattle, WA (US); SEATTLE CHILDREN'S RESEARCH INSTITUTE, Seattle, WA (US); COMMONWEALTH SCIENTIFIC AND INDUSTRIAL RESEARCH ORGANISATION, Acton, Australian Capitol Territory (AU)

(21) Appl. No.: 16/491,869

(22) PCT Filed: Mar. 6, 2018
(86) PCT No.: PCT/US18/21201

§ 371 Date: Sep. 6, 2019

Related U.S. Application Data
(60) Provisional application No. 62/467,667, filed on Mar. 6, 2017.

(51) Int. Cl. A61K 47/58; A61K 35/17; A61K 35/15; A61K 47/69; A61K 47/54; A61K 31/4453

(52) U.S. Cl. CPC A61K 47/58; A61K 35/17; A61K 31/4453; A61K 47/6901; A61K 47/545; A61K 35/15

(57) ABSTRACT

Provided herein are engineered cells and methods for engineering cells to deliver a therapeutic agent, e.g., a small molecule, peptide or other drug, to a cell or tissue to be treated.

### CROSS-REFERENCE TO RELATED APPLICATION

This application claims benefit under 35 U.S.C. § 119(e) of the U.S. Provisional Application No. 62/467,667 filed Mar. 6, 2017, the contents of which are incorporated herein by reference in their entirety.

### GOVERNMENT SUPPORT

This invention was made with government support under Grant No. HDTRA1-13-1-0047, awarded by the Defense Threat Reduction Agency. The government has certain rights in the invention.

### FIELD OF THE INVENTION

The field of the invention relates generally to drug delivery; particular embodiments relate to the treatment of autoimmune disease, infectious disease and/or cancer.

### BACKGROUND

Small molecule drugs and other therapeutic agents are most often delivered systemically, e.g., by oral or IV administration, and a variety of approaches and formulations for sustained delivery or controlled release are known in the art. These can include polymers that essentially entrap the drug and release it over time as the polymer naturally degrades. Localized delivery of therapeutic agents using these approaches relies, most often, on injection at a given site, or implantation of a delivery device or therapeutic agent depot at or near the desired site of action. Localized delivery can have advantages over systemic delivery in achieving a high local concentration of drug while minimizing potential for systemic side effects. Often the total amount of therapeutic agent required to be administered is considerably smaller than when systemic administration is used. However, such delivery using conventional approaches is often invasive and relies upon detailed knowledge of the location to be targeted—when the target is dispersed, e.g., as for tumor metastases, the precise number and location of target areas are not necessarily known.

Adoptive cell transfer (“ACT”) refers to the process of treating disease in a patient by infusing autologous or allogeneic cells of various cell lineages to treat disease. For example, hematopoietic stem cell (“HSC”) transplantation involves the infusion of autologous or allogeneic stem cells to reestablish hematopoietic function in patients whose bone marrow or immune system is damaged or defective. It also allows the introduction of genetically modified HSCs, for example to treat congenital genetic diseases. In typical HSC transplantation, the HSCs are obtained from the bone marrow, peripheral blood or umbilical cord blood.

Another example of adoptive cell transfer is the infusion of autologous or allogeneic T-cells that are selected and/or engineered ex vivo to target specific antigens (e.g., tumor-associated antigens). The T lymphocytes are typically obtained from the peripheral blood of the donor by leukapheresis. In some T cell immunotherapy methods, donor T-lymphocytes are engineered ex vivo to express chimeric antigen receptors (“CAR”s) of predetermined specificity. CARs typically include an extracellular domain, such as the binding domain from an scFv, that confers specificity for a desired antigen, a transmembrane domain, and/or an intracellular domain(s) that trigger T-cell effector functions (e.g., CD28 and/or 4-IBB (Jensen and Riddell, Immunological Reviews 257: 127-144 (2014)). In still other T cell immunotherapy methods, T lymphocytes obtained from the donor are engineered ex vivo to express T cell receptors (“TCR”s) that confer desired specificity for antigen presented in the context of specific HLA alleles (Liddy et al, Nat. Med. 18(6):980-988 (2012)).

### SUMMARY

The methods, compositions and treatments described herein provide improved treatment of diseases, such as cancer, infection and autoimmune disease by modifying cells to comprise and deliver at least one agent that is effective against the disease. Such methods, compositions and treatments take advantage of, among other things, cells' ability to home to a particular location for delivery of an agent, and cells' capacity to effect treatment results in addition to or in synergy with the effect of the agent delivered. The methods, compositions and treatments described herein rely, in part, upon the use of drug copolymer compositions (alternately referred to herein as “drugamer” compositions) that bear ligands that permit their association with cells bearing cell-surface polypeptides that bind those ligands. In this manner, cells loaded with or carrying drug copolymer compositions are generated, which can, in some embodiments, track to a desired location and deliver drug from the drug copolymer composition at that location. In certain embodiments, described more particularly in the following, the cells are engineered to express further heterologous polypeptides that assist in targeting the cell to a given location or microenvironment, or in providing another beneficial or therapeutic function to the cell, or both.

In one aspect, provided herein is a composition comprising: a) a genetically engineered cell that expresses on its cell surface at least one heterologous ligand-binding polypeptide; and b) at least one copolymer drug composition, wherein each of the at least one copolymer drug composition comprises a ligand that specifically binds the heterologous ligand-binding polypeptide described in (a), such that the at least one copolymer drug composition is displayed on the surface of the genetically engineered cell, wherein the engineered cell further comprises a nucleic acid construct encoding a polypeptide, operably linked to a regulatory nucleic acid sequence that renders expression of the polypeptide modulated by the presence or absence of the at least one drug comprised by the copolymer drug composition.

In an alternative aspect, provided herein is a composition comprising: a) a genetically engineered cell that expresses on its cell surface at least one heterologous ligand-binding polypeptide; and b) a first copolymer drug composition, wherein each of the first copolymer drug composition comprises a ligand that specifically binds the heterologous ligand-binding polypeptide described in (a), such that the first copolymer drug composition is displayed on the surface of the genetically engineered cell, wherein the engineered cell further comprises a nucleic acid construct encoding a polypeptide, operably linked to a regulatory nucleic acid sequence that renders expression of the polypeptide modulated by the presence or absence of the first drug comprised by the first copolymer drug composition.

In one embodiment of this aspect and all other aspects described herein, the genetically engineered cell further comprises a second copolymer drug composition.

In another embodiment of this aspect and all other aspects provided herein, the first drug comprised by the first copolymer drug composition is different from the second drug comprised by the second copolymer drug composition.

In one embodiment of this aspect and all other aspects described herein, the second drug does not bind to or act on the regulatory nucleic acid sequence.

In another embodiment of this aspect and all other aspects provided herein, the second drug comprises a therapeutic agent that acts on a target cell (e.g., a cancer cell, a macrophage), a cellular microenvironment (e.g., tumor microenvironment), etc.

In one embodiment of this aspect and all other aspects described herein, the at least one heterologous ligand-binding polypeptide comprises an antigen binding domain of an antibody that binds the ligand comprised by a copolymer drug composition.

In one embodiment of this aspect and all other aspects described herein, the genetically-engineered cell further expresses a heterologous receptor that binds a cell-surface ligand on a target cell.

While applicable to any of a wide range of cell types, in another embodiment of this aspect and all other aspects described herein, the genetically engineered cell is a T cell, a macrophage or a stem cell.

In another embodiment of this aspect and all other aspects described herein, the stem cell is a hematopoietic stem cell or a neuronal stem cell.

In another embodiment of this aspect and all other aspects described herein, the heterologous receptor that binds a cell-surface ligand on a target cell binds a tumor antigen expressed on a target cell.

In another embodiment of this aspect and all other aspects described herein, the heterologous receptor that binds a cell-surface ligand on a target cell comprises a chimeric T cell receptor.

In another embodiment of this aspect and all other aspects described herein, the heterologous receptor that binds a cell-surface ligand on a target cell comprises the antigen-binding domain of an antibody.

In another embodiment of this aspect and all other aspects described herein, the drug comprised by the at least one copolymer drug composition comprises a small molecule drug.

In another embodiment of this aspect and all other aspects described herein, the drug comprised by the copolymer drug composition is selected from the group consisting of doxycycline, and tetracycline.

In another embodiment of this aspect and all other aspects described herein, the drug comprised by the copolymer drug composition is required for the expression of the polypeptide

In another embodiment of this aspect and all other aspects described herein, the copolymer drug composition comprises 4-hydroxytamoxifen, and/or CMP-8.

In another embodiment of this aspect and all other aspects described herein, the nucleic acid construct comprises a sequence encoding a riboswitch that responds to a drug comprised by the copolymer drug composition. Riboswitch-mediated gene regulation involves direct small molecule target recognition and binding by an RNA, where target binding modulates the function, e.g., translation activity of the RNA. Riboswitches are reviewed, for example, in Sherwood & Herkin, Ann. Rev. Microbiol. 70:361-374 (2016).

In another embodiment of this aspect and all other aspects described herein, the nucleic acid construct is regulated by chemically-induced dimerization, and wherein the copolymer drug composition comprises a chemical inducer of the chemically-induced dimerization.

In other embodiments of this aspect and all other aspects described herein: a) the chemically-induced dimerization comprises FKBP homodimerization and the copolymer drug composition comprises FK1012; b) the chemically-induced dimerization comprises FKBP dimerization with Calcineurin A and the copolymer drug composition comprises FK506; c) the chemically-induced dimerization comprises FKBP dimerization with CyP-Fas and the copolymer drug composition comprises FKCsA; d) the chemically-induced dimerization comprises FKBP dimerization with the FRB domain of mTOR and the copolymer drug composition comprises rapamycin; e) the chemically-induced dimerization comprises GyrB homodimerization and the copolymer drug composition comprises couermycin; or f) the chemically-induced dimerization comprises GAI heterodimerization with GID1 and the copolymer drug composition comprises gibberellin. Other examples are known to those of skill in the art.

In another embodiment of this aspect and all other aspects described herein, the expression of the polypeptide is repressed by the drug comprised by the copolymer drug composition.

In another embodiment of this aspect and all other aspects described herein, the polypeptide promotes the death of the genetically engineered cell. In this manner, the cell can be selectively eliminated after it has served its therapeutic purpose.

In another embodiment of this aspect and all other aspects described herein, the polypeptide modulates an activity of a target cell. In one embodiment, the polypeptide comprises an immunomodulator, an inhibitor of a growth factor or a growth factor receptor.

In another embodiment of this aspect and all other aspects described herein, the immunomodulator comprises an immune checkpoint inhibitor, a cytokine, a chemokine, or a polypeptide that influences macrophage or T cell polarization.

In another embodiment, the immunomodulator comprises an inhibitor of an immune checkpoint polypeptide selected from, for example, PD-1, PD-L1, TIM-3, CTLA4, TIGIT, KIR, LAG3, and/or DD1-α.

In another embodiment, the immunomodulator comprises a cytokine or chemokine. In one embodiment, the cytokine or chemokine is selected from the group consisting of: IL-1, IL-6, IL-7, IL-12, IL-15, IL-17, IL-18, IL-21, IL-23, GM-CSF, TNFa, Type I and II interferons. Other immunomodulators include, for example, checkpoint blockades (PD-1, CTLA-4, B7-H4), CD28 agonist, 41BBL, and 2B4. As but one example, a cytokine expressed by the cell can include IL-15, which supports T cell activity. When used in combination with an anti-tumor drug carried by a drug copolymer composition, such a cell can promote immune attack on the tumor while attacking one or more additional tumor growth pathways. Other immunomodulators that can be expressed by the cell carrying the drug copolymer composition can include, for example, TLR agonists, e.g., agonists of TLR5, TLR7 and/or TLR8. A combination of expression of TLR5 or a TLR5 agonist with delivery of, e.g., a small molecule TGF-β inhibitor via a drug copolymer composition is also contemplated for additive or synergistic effects in tumor therapy.

In another embodiment of this aspect and all other aspects described herein, the polypeptide comprises an antigen-binding domain of an antibody.

In another embodiment of this aspect and all other aspects described herein, the composition comprises at least two different copolymer drug compositions. In this instance, one of the copolymer drugs can regulate expression of a transgene, and the other can, for example, treat a target disease or disorder. Thus, in one embodiment, at least one of the at least two different copolymer drug compositions comprises a kinase inhibitor, a growth factor receptor inhibitor, a chemotherapeutic, an anti-inflammatory agent, an immunosuppressant, an estrogen receptor (ER) ligand, a Toll-Like Receptor (TLR) antagonist, an indoleamide 2,3dioxygenase inhibitor, a TGFβ receptor I (TβRI) inhibitor, a corticosteroid (e.g., betamethasone, budesonide, cortisone, dexamethasone, hydrocortisone, methylprednisolone, prednisolone, and/or prednisone) and a cyclic dinucleotides (CDNs) STING agonist.

In another embodiment of this aspect and all other aspects described herein, the composition comprises at least two different copolymer compositions as recited in (b).

In another embodiment of this aspect and all other aspects provided herein, only one of the at least two different copolymer drugs binds to the regulatory nucleic acid sequence.

In another embodiment of this aspect and all other aspects provided herein, another of the at least two different copolymer drugs is a therapeutic agent.

In another embodiment of this aspect and all other aspects provided herein, one of the at least two different copolymer drugs is a therapeutic agent.

In another embodiment of this aspect and all other aspects described herein, wherein the composition further comprises a gel or matrix comprising one or more agents that acts upon the genetically engineered cell or a target cell thereof.

Another aspect provided herein relates to a method of administering a polypeptide of interest to an individual in need thereof, the method comprising administering to the individual a composition as described herein, wherein a drug comprised by the copolymer drug composition is released from the copolymer after the composition is administered, and wherein that drug induces expression of the polypeptide such that the expression of the polypeptide continues only while that drug is present.

In one embodiment of this aspect and all other aspects described herein, the at least one heterologous ligand-binding polypeptide comprises an antigen binding domain of an antibody that binds the ligand comprised by a copolymer drug composition.

In another embodiment of this aspect and all other aspects described herein, the genetically engineered cell is contacted with the at least one copolymer drug composition before the genetically engineered cell is administered.

In another embodiment of this aspect and all other aspects described herein, the genetically-engineered cell further expresses a heterologous receptor that binds a cell-surface ligand on a target cell.

In another embodiment of this aspect and all other aspects described herein, the target cell is a tumor cell and wherein the polypeptide of interest promotes tumor cell death.

In another embodiment of this aspect and all other aspects described herein, the polypeptide of interest comprises a toxin, an immunomodulator, a TRAIL polypeptide, or an inhibitor of a growth factor or growth factor receptor.

In another embodiment of this aspect and all other aspects described herein, the cell comprises at least two different copolymer drug compositions. In one embodiment, at least one of the at least two different copolymer drug compositions comprises a kinase inhibitor, a growth factor receptor inhibitor, a chemotherapeutic, an anti-inflammatory agent, an immunosuppressant, an estrogen receptor (ER) ligand, a Toll-Like Receptor (TLR) antagonist, an indoleamide 2,3dioxygenase inhibitor, a TGFβ receptor I (TβRI) inhibitor, a corticosteroid (e.g., betamethasone, budesonide, cortisone, dexamethasone, hydrocortisone, methylprednisolone, prednisolone, and/or prednisone), and a cyclic dinucleotides (CDNs) STING agonist.

While applicable to any of a wide range of cell types, in another embodiment, the genetically engineered cell is a T cell, a macrophage or a stem cell.

In another embodiment of this aspect and all other aspects described herein, the stem cell is a hematopoietic stem cell or a neuronal stem cell.

In another embodiment of this aspect and all other aspects described herein, the heterologous receptor that binds a cell-surface ligand on a target cell binds a tumor antigen expressed on a target tumor cell.

In another embodiment of this aspect and all other aspects described herein, the heterologous receptor that binds a cell-surface ligand on a target cell comprises a chimeric T cell receptor.

In another embodiment of this aspect and all other aspects described herein, the heterologous receptor that binds a cell-surface ligand on a target cell comprises the antigen-binding domain of an antibody.

In another embodiment of this aspect and all other aspects described herein, the at least one copolymer drug composition comprises a small molecule drug.

In another embodiment of this aspect and all other aspects described herein, the small molecule drug is selected from the group consisting of doxycycline, and tetracycline.

In another embodiment of this aspect and all other aspects described herein, a drug comprised by the copolymer drug composition is required for the expression of the polypeptide of interest.

In another embodiment of this aspect and all other aspects described herein, the polypeptide of interest modulates an activity of a target cell.

In another embodiment of this aspect and all other aspects described herein, the polypeptide of interest comprises an immunomodulator, an inhibitor of a growth factor or a growth factor receptor.

In another embodiment of this aspect and all other aspects described herein, the immunomodulator comprises an immune checkpoint inhibitor, a cytokine, a chemokine, a polypeptide that influences macrophage or T cell polarization.

In another embodiment of this aspect and all other aspects described herein, the immunomodulator comprises an inhibitor of an immune checkpoint polypeptide, for example, selected from the group consisting of PD-1, PD-L1, TIM-3, CTLA4, TIGIT, KIR, LAG3, and DD1-α.

In another embodiment, the immunomodulator comprises a cytokine or chemokine. In one embodiment, the cytokine or chemokine is selected from the group consisting of: IL-1, IL-6, IL-7, IL-12, IL-15, IL-17, IL-18, IL-21, IL-23, GM-CSF, TNFa, Type I and II interferons. Other immunomodulators include, for example, checkpoint blockades (PD-1, CTLA-4, B7-H4), CD28 agonist, 41BBL, and 2B4. As but one example, a cytokine expressed by the cell can include IL-15, which supports T cell activity. When used in combination with an anti-tumor drug carried by a drug copolymer composition, such a cell can promote immune attack on the tumor while attacking one or more additional tumor growth pathways. Other immunomodulators that can be expressed by the cell carrying the drug copolymer composition can include, for example, TLR agonists, e.g., agonists of TLR5, TLR7 and/or TLR8. A combination of expression of TLR5 or a TLR5 agonist with delivery of, e.g., a small molecule TGF-β inhibitor via a drug copolymer composition is also contemplated for additive or synergistic effects in tumor therapy.

In another embodiment of this aspect and all other aspects described herein, the polypeptide of interest comprises an antigen-binding domain of an antibody.

In another embodiment of this aspect and all other aspects described herein, the composition comprises at least two different copolymer compositions as recited in (b).

In another embodiment of this aspect and all other aspects provided herein, only one of the at least two different copolymer drugs binds to the regulatory nucleic acid sequence.

In another embodiment of this aspect and all other aspects provided herein, another of the at least two different copolymer drugs is a therapeutic agent.

In another embodiment of this aspect and all other aspects provided herein, one of the at least two different copolymer drugs is a therapeutic agent.

In another embodiment of this aspect and all other aspects provided herein, the therapeutic agent comprises a kinase inhibitor, a growth factor receptor inhibitor, a chemotherapeutic, an estrogen receptor (ER) ligand, a Toll-like Receptor (TLR) antagonist, an indoleamide 2,3-dioxygenase inhibitor, a TGF-beta (TβR1) inhibitor, and a cyclic dinucleotides (CDNs) STING agonist.

In another embodiment of this aspect and all other aspects described herein, the genetically engineered cell is administered in a gel or matrix comprising one or more agents that acts upon the genetically engineered cell or a target cell thereof.

Also provided herein, in another aspect, is a method of limiting the duration of a cell-mediated therapy in an individual in need thereof, the method comprising administering to the individual a composition comprising: a) a genetically engineered cell that expresses on its cell surface at least one heterologous ligand-binding polypeptide; and b) at least one copolymer drug composition, wherein each of the at least one copolymer drug composition comprises a ligand that specifically binds a heterologous ligand-binding polypeptide described in (a), such that the at least one copolymer drug composition is displayed on the surface of the genetically engineered cell, wherein the engineered cell further comprises a nucleic acid construct encoding a first polypeptide of interest, operably linked to a regulatory nucleic acid sequence that renders expression of the first polypeptide of interest sensitive to the presence or absence of a drug comprised by a copolymer drug composition described in (b), wherein the engineered cell further comprises a nucleic acid construct encoding a second polypeptide of interest, wherein a drug comprised by a copolymer drug composition as described in (b) is released from the copolymer after the composition is administered, wherein a drug released from a copolymer composition represses the expression of the first polypeptide of interest, wherein the second polypeptide of interest is a therapeutic polypeptide, and wherein the first polypeptide of interest promotes the death of the genetically engineered cell.

In one embodiment of this aspect and all other aspects described herein, the first polypeptide comprises a toxin polypeptide.

In another embodiment of this aspect and all other aspects described herein, the toxin polypeptide comprises a restriction endonuclease, a cytolytic peptide, Apoptosis-Inducing Factor (AIF), a caspase polypeptide, or diphtheria toxin A fragment.

In another embodiment of this aspect and all other aspects described herein, the drug that represses expression of the first polypeptide is selected from the group consisting of tetracycline, and doxycycline.

Another aspect provided herein relates to a composition comprising: a) a genetically engineered cell that expresses on its cell surface at least one heterologous ligand-binding polypeptide; and b) a first copolymer drug composition, wherein the first copolymer drug composition comprises a ligand that specifically binds the heterologous ligand-binding polypeptide described in (a), such that the first copolymer drug composition is displayed on the surface of the genetically engineered cell, wherein the engineered cell further comprises a nucleic acid construct encoding a polypeptide, operably linked to a regulatory nucleic acid sequence that renders expression of the polypeptide modulated by the presence or absence of a first drug comprised by the first copolymer drug composition.

In one embodiment of this aspect and all other aspects provided herein, the genetically engineered cell further comprises a second copolymer drug composition.

In another embodiment of this aspect and all other aspects provided herein, the first drug comprised by the first copolymer drug composition is different from the second drug comprised by the second copolymer drug composition.

In another embodiment of this aspect and all other aspects provided herein, second drug does not bind to or act on the regulatory nucleic acid sequence.

In another embodiment of this aspect and all other aspects provided herein, the second drug comprises a therapeutic agent that acts on a target cell (e.g., a cancer cell, a macrophage), a cellular microenvironment (e.g., tumor microenvironment), etc.

### Definitions

The terms “heterologous ligand-binding polypeptide” and “heterologous receptor that binds a cell-surface ligand” are used herein to refer to classes of polypeptide that have similar characteristics, but perform different functions in the context of the methods and compositions described herein. Each of these polypeptides is expressed from a recombinant nucleic acid construct introduced to the cell and is heterologous to the cell on which it is expressed, in that it is not normally expressed on the surface of the cell to which it is introduced, or, at a minimum, is not normally expressed on the surface of such a cell at the level driven by the recombinant construct. Each of these polypeptides includes a domain that specifically binds a target ligand, and this domain in each of these polypeptides can include, but is not limited to an antigen-binding domain of an antibody, most often, but not necessarily, an scFv. A “heterologous ligand-binding polypeptide” as the term is used herein, includes a ligand-binding domain that specifically binds a cognate ligand molecule included in a copolymer drug composition as described herein. In this manner, a cell engineered to express such a ligand-binding polypeptide on its surface will bind and display the copolymer drug composition that includes its cognate ligand. A “heterologous receptor that binds a cell-surface ligand,” as the term is used herein, is expressed on a cell engineered to express a heterologous ligand-binding polypeptide as described herein, and binds a ligand expressed on the surface of another cell. The heterologous receptor that binds a cell-surface ligand can permit the localization and binding of the cell that expresses it to a particular target cell in a chosen microenvironment, e.g., a tumor microenvironment. This approach can facilitate localization of an engineered cell as described herein to any of a wide variety of target cell types; however, in one embodiment, the heterologous receptor binds a tumor antigen and mediates binding of the cell to a tumor cell. In this manner, a drug included in a copolymer drug composition as described herein can be delivered to a targeted location, including but not limited to a tumor location. In one embodiment, the heterologous receptor includes a T cell receptor, or a chimeric antigen receptor, e.g., a chimeric T cell antigen receptor or CAR. Further considerations for heterologous ligand-binding polypeptides and heterologous receptors as those terms are used herein are discussed herein below.

As used herein, the term “copolymer drug composition” refers to a composition comprising a copolymer and at least one small molecule drug or therapeutic agent bound to the copolymer that effectively delivers its drug or therapeutic agent by timely release of the drug or therapeutic agent.

The term “ligand” as used herein refers to a molecule or substrate that binds specifically to e.g., a receptor to form a complex and/or target another substance. Examples of ligands include epitopes on antigens, or molecules that bind to receptors, substrates, inhibitors, hormones, and activators. “Ligand binding domain” as the term is used herein refers to a region or portion of e.g., a receptor that recognizes and binds to a certain ligand. Examples of ligand binding domains include antigen binding portions of antibodies, extracellular domains of receptors, and active sites of enzymes. Where a ligand necessarily binds to a binding partner (e.g., to a receptor), one can also consider any ligand as a member of a ligand:ligand binding partner (e.g., ligand:receptor) pair.

“Chimeric receptor” as used herein refers to a synthetically designed receptor comprising a ligand binding domain of an antibody or other protein sequence that binds to a cell-surface molecule on a target cell (e.g., a cancer cell or a virally-infected cell, among others) and is linked via a spacer domain to one or more intracellular signaling domains of a T cell or other receptors, such as a costimulatory domain. A chimeric receptor can also be referred to as an artificial T cell receptor, chimeric T cell receptor, chimeric immunoreceptor, or chimeric antigen receptor (CAR). These provide engineered receptors that can graft an arbitrary specificity onto an immune cell receptor. Chimeric antigen receptors are considered by some investigators to include the antibody or antibody fragment, the spacer, signaling domain, and transmembrane region. However, due to the surprising effects of modifying the different components or domains of the CAR, such as the epitope binding region (for example, antibody fragment, scFv, or portion thereof), spacer, transmembrane domain, and/or signaling domain), in some contexts, in the present disclosure, the components of the CAR are described independently. The variation of the different elements of the CAR can, for example, lead to stronger binding affinity for a specific epitope.

These artificial T-cell receptors, or CARs, can be used in a therapy for cancer or a viral infection using adoptive cell transfer. In this process, T-cells are removed from a patient and modified so that they express receptors specific for a molecule displayed on a cancer cell or a virus, or a virus-infected cell. The genetically engineered T-cells, which can then recognize and kill the cancer cells or the virus infected cells or promote clearance of the virus, are reintroduced into the patient. In some embodiments, a method of treating, inhibiting, or ameliorating a disease in a subject in need thereof is provided. In other embodiments, a method of augmenting an immune response to a desired target (e.g., tumor microenvironment, cancer cell, microbe, fungi etc.) is provided.

A “subject” or subjects that can be provided the compositions described herein includes humans and other primate subjects, such as monkeys and apes for veterinary medicine purposes; however, the technology is also contemplated for use with domestic animals, such as horses, pigs, sheep, cattle, and goats, as well as, companion animals, such as dogs and cats. The subjects can be male or female and can be of any suitable age, including infant, juvenile, adolescent, adult, and geriatric subjects.

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

As used herein, the term “specifically binds” refers to the ability of a ligand-binding molecule or a receptor to bind a ligand in a selective manner. A ligand-binding molecule or receptor specifically binds a ligand if the dissociation constant, K_{D} is 10⁻⁵ M or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of a ligand binding molecule for its ligand, and their relative concentrations. The person of ordinary skill in the art can determine appropriate conditions under which ligand binding molecules as described herein selectively bind their target ligands using any suitable methods, such as titration of a polypeptide agent in a suitable cell binding assay. A ligand-binding molecule specifically bound to a target ligand is not displaced by a non-similar competitor. In certain embodiments, an antibody, antigen-binding portion thereof, or CAR is said to specifically bind an antigen when it preferentially recognizes its target antigen in a complex mixture of proteins and/or macromolecules.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIGS. 1A-1D Schematic representations of exemplary compositions described herein including: a targeted cell comprising a drugamer (i.e., a drug/polymer complex) (FIG. 1A), a genetically modified and targeted cell comprising a drugamer (FIG. 1B), an exemplary embodiment of a composition as described herein for the treatment of a tumor (FIG. 1C), and incorporation of a cell composition as described herein in a scaffold (FIG. 1D).

FIGS. 2A-2D An exemplary anti-cancer agent (CMP8) copolymerized with solubilizing carboxybetaine and an engineered cell receptor targeting fluorescein moiety (FIG. 2A). FIG. 2B, An exemplary synthesis protocol for the composition depicted in FIG. 2A. FIG. 2C, An exemplary ¹H NMR spectrum of the composition in FIG. 2A to show formation of the desired product. FIG. 2D, Mole and weight percentage calculations based on NMR for the composition of FIG. 2A.

FIGS. 3A-3B A schematic representation and data showing binding of a drugamer to genetically engineered macrophages (GEMs) expressing anti-fluorescein receptor is shown in FIG. 3A.

FIG. 3B shows data that indicate macrophages retain payload on the surface for 72 hours, although MFI is reduced.

FIGS. 4A-4C An illustration of a synthesis protocol for Fluorescein monomer (FIG. 4A). An exemplary ¹H-NMR spectrum of Fluorescein monomer is shown in FIG. 4B. ESI-Mass spectrum of Fluorescein monomer data is shown in FIG. 4C.

FIG. 5 An exemplary ¹H NMR spectrum of a carboxybetaine monomer (protected form).

FIGS. 6A-6B A Schematic illustration of an exemplary method for Rhodamine-HEMA monomer synthesis is shown in FIG. 6A. An exemplary ¹H-NMR spectrum of Rhodamine monomer is shown in FIG. 6B.

FIGS. 7A-7D A schematic illustration of an exemplary synthesis protocol for galunisertib monomer. FIG. 7A shows the chemical structures depicting synthesis of galunisertib. FIG. 7B shows ¹H-NMR of galunisertib monomer. FIG. 7C ¹³C-NMR (100 MHz) of galunisertib monomer. FIG. 7D ESI-MS of galunisertib monomer.

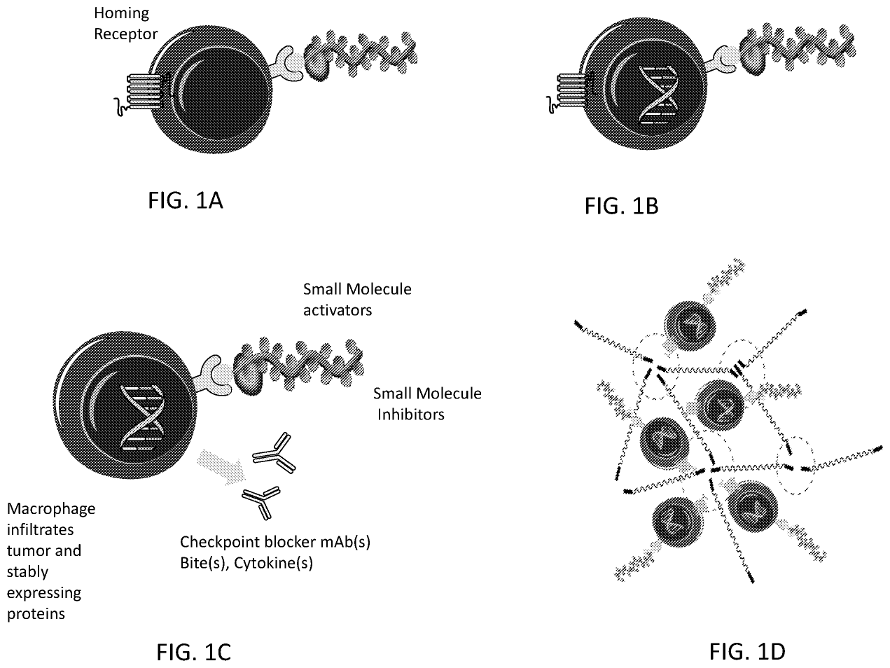

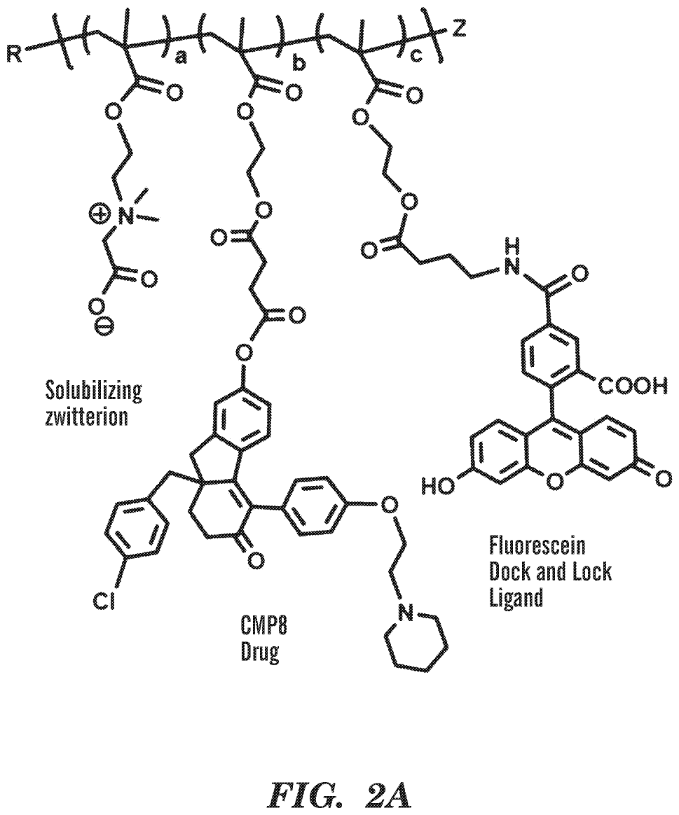

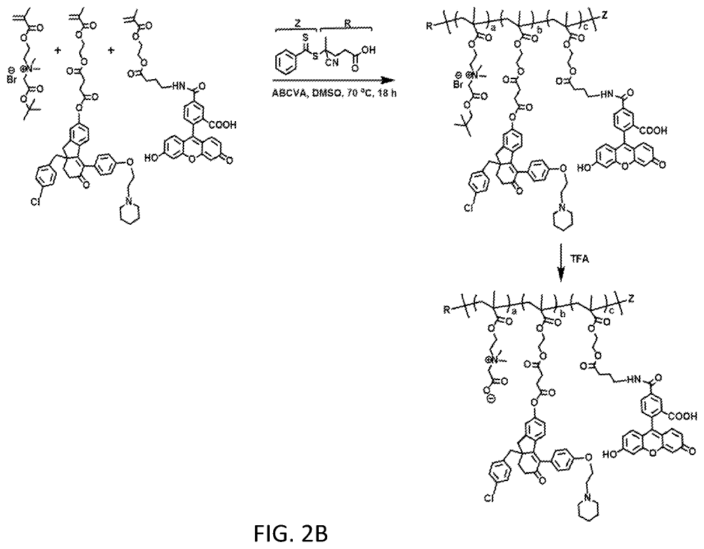

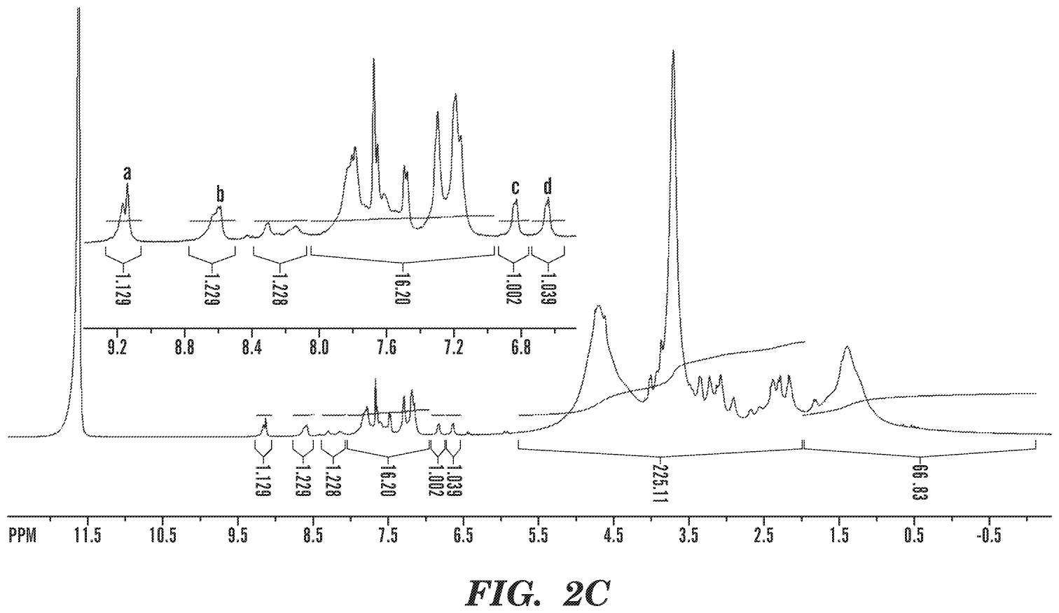

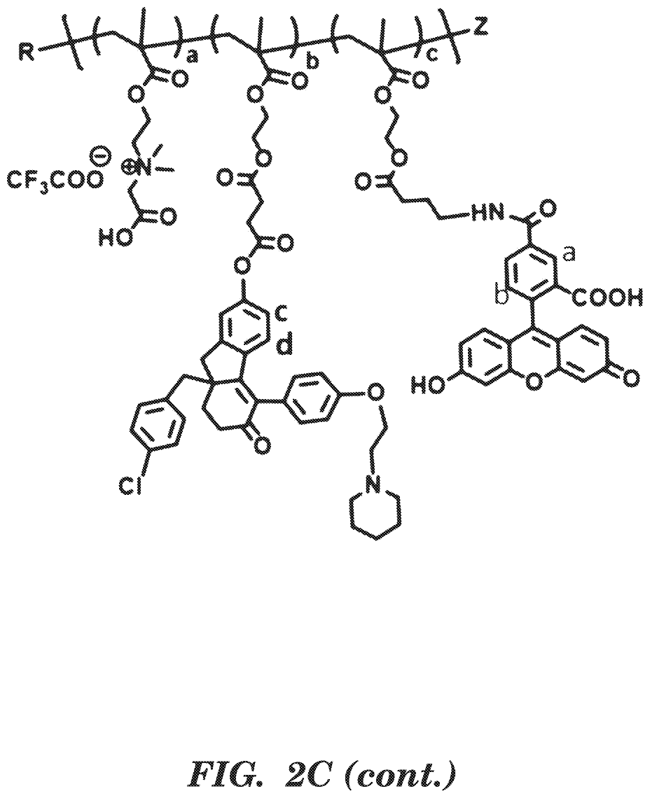

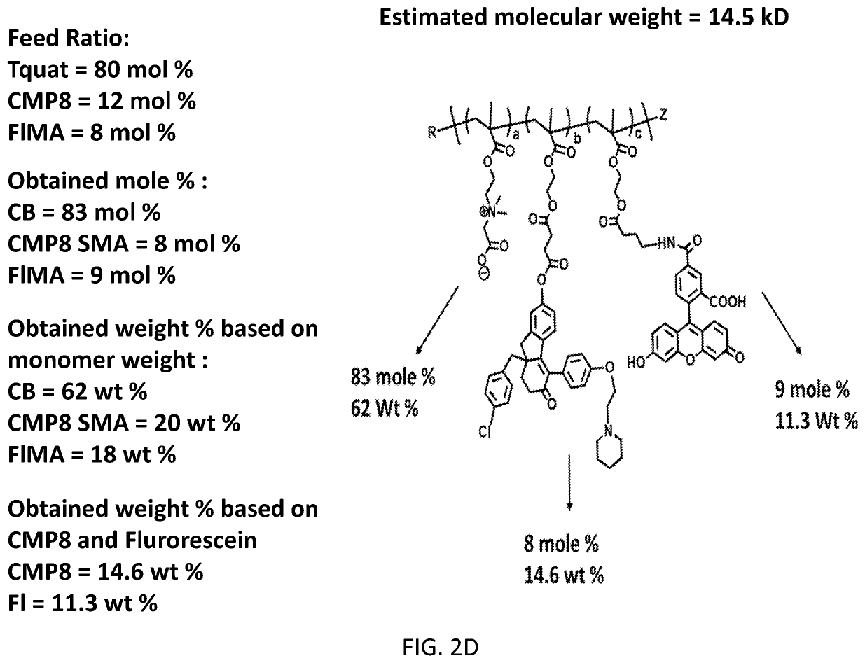

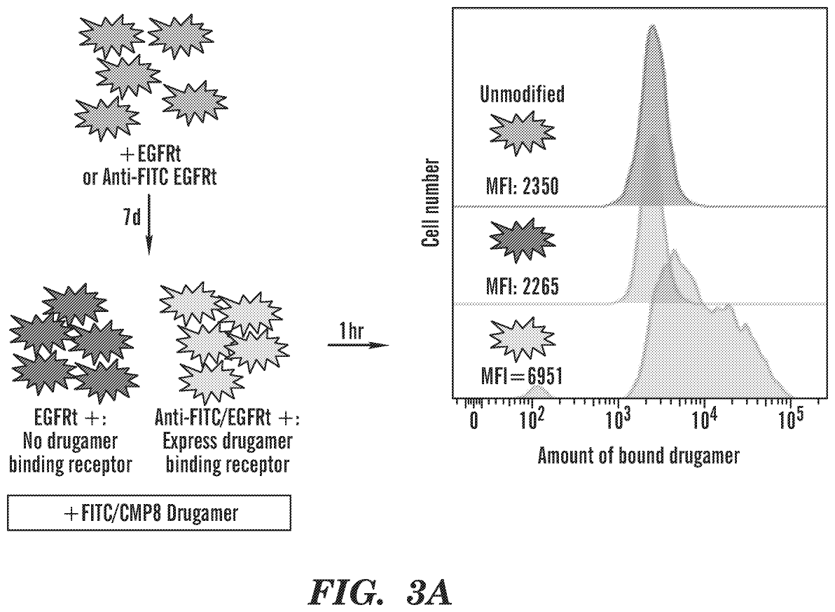

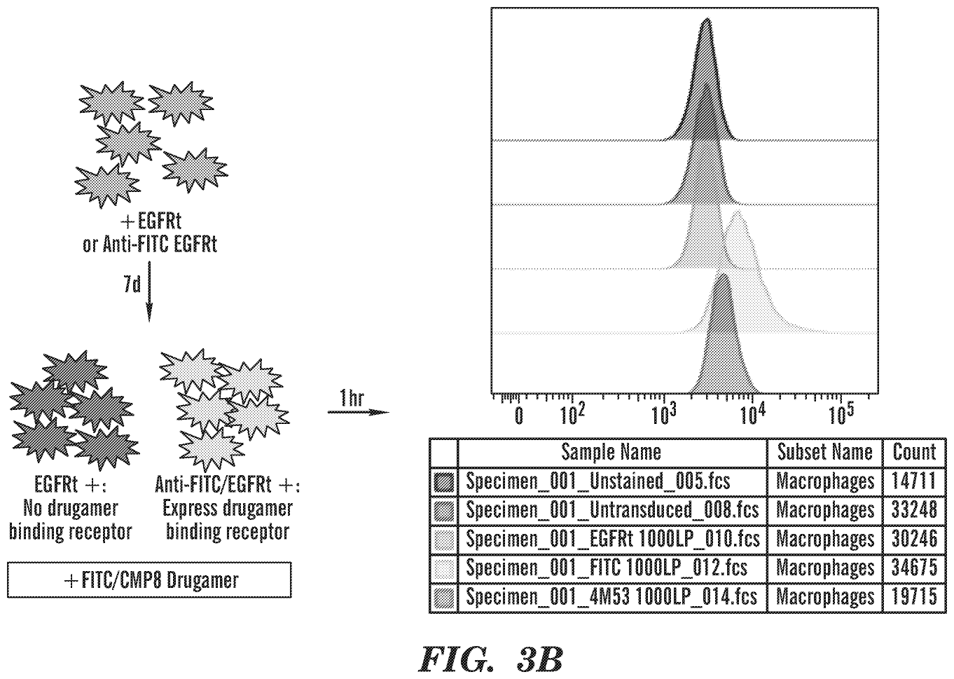

### DETAILED DESCRIPTION

Provided herein are engineered cells and methods for engineering cells to deliver a therapeutic agent, e.g., a small molecule, peptide or other drug, to a cell or tissue to be treated. The methods and compositions described herein, in part, take advantage of the ability of cells to home to or be directed to a desired location in the body and thereby achieve localized effects that minimize the potential for off-target effects of the therapeutic agent. When the cell itself has or is otherwise engineered to have its own therapeutic effect, e.g., through immune effector activity or stimulation of such activity as but one example, engineered cells as described herein can add to or synergize with the therapeutic agent's effect to treat a targeted indication. In some embodiments, a cell composition as described herein comprises a cell genetically engineered to express a desired gene product, and a therapeutic agent, wherein the presence of the therapeutic agent drives the expression of the desired gene product. In one embodiment, expression of the desired gene product can, in turn, act on the cell itself to induce a desired function or cell phenotype. Once the release of the therapeutic agent from the cell is exhausted, the desired gene product is no longer expressed. Such compositions permit the delivery of a desired gene product in an inducible and/or self-limiting manner, thereby expanding the utility of the compositions to, for example, short-term treatment regimens etc. Guidance and considerations necessary to practice this technology are set out in the following.

### Therapeutic Agent Delivery Using Cells Carrying Copolymer Drug Compositions

In one embodiment, the compositions and methods described herein relate to cells modified to carry drug-containing copolymers. In the following, the drug copolymer compositions (or, alternatively, “copolymer drug compositions”) are described, as are the cells and methods of using them to treat disease.

### Drug Copolymer Compositions

The drug copolymer compositions useful in the methods and compositions described herein advantageously have high therapeutic agent content and therefore are powerful as therapeutic agent-dense delivery systems. The drug copolymer compositions are also advantageously stable to physiological conditions encountered in the circulatory system and deliver their cargo (e.g., a small molecule drug) at effective release rates.

The high therapeutic agent density of the drug copolymer compositions results from the methods used in preparing the carriers. In some embodiments, the drug copolymer compositions are prepared by conventional conjugation processes involving conjugation of a version of the therapeutic agent, including a pro-drug, to a pre-formed polymer having a plurality of pendant side chains comprising reactive groups. In other embodiments, the drug copolymer compositions are prepared by polymerization processes that include copolymerization of a polymerizable prodrug monomer with one or more other monomers. By virtue of introducing the therapeutic agent into the drug copolymer compositions by polymerization of a polymerizable prodrug monomer, the drug copolymer compositions described herein offer significantly greater therapeutic agent density compared to conventional polymeric drug carriers.

In some embodiments, in addition to constitutional units that include releasable therapeutic agents, the drug copolymer compositions useful in the methods and compositions described herein also include constitutional units that include stabilizing groups. The stabilizing groups are hydrophilic groups that are readily hydrated under physiological conditions. The stabilizing groups include uncharged hydrophilic groups and substantially electronically neutral groups. As discussed in detail below, uncharged hydrophilic groups include polyether groups, such as poly(alkylene oxide)s (e.g., poly(ethylene oxide), PEG) and polyhydroxyl groups, such as saccharides (e.g., mono- and polysaccharides); and substantially electronically neutral groups include zwitterionic groups (carboxy-, sulfo- and phosphobetaines) and ampholyte groups (constitutional units that include positively charged groups or groups that become positively charged under physiological conditions, and constitutional units that include negatively charged groups or groups that become negatively charged under physiological conditions). Like the incorporation of the therapeutic agents, the stabilizing groups are introduced into the drug copolymer compositions, as described herein, by polymerization processes that involve copolymerization of a suitable stabilizing group monomer with a polymerizable prodrug monomer.

Generally, the various polymers included as constituent moieties of the compounds described herein can comprise one or more repeat units-monomer (or monomeric) residues-derived from a process which includes polymerization. Such monomeric residues can optionally also include structural moieties (or species) derived from post-polymerization (e.g., derivatization) reactions. Monomeric residues are constituent moieties of the polymers, and accordingly, can be considered as constitutional units of the polymers. Generally, a polymer as described herein can comprise constitutional units which are derived (directly or indirectly via additional processes) from one or more polymerizable monomers.

The polymer can be a copolymer, derived from polymerization of two or more different monomers having different chemical compositions. Polymers which are copolymers include random copolymer chains (e.g., terpolymers) or block copolymer chains (e.g., diblock copolymer, triblock copolymer, higher-ordered block copolymer, etc.). Any given block copolymer chain can be conventionally configured and effected according to methods known in the art.

In some embodiments, the polymer is a linear polymer, or a non-linear polymer. Non-linear polymers can have various architectures, including for example branched polymers, brush polymers, star-polymers, dendrimer polymers, and can be cross-linked polymers, semi-cross-linked polymers, graft polymers, and combinations thereof.

Polymerization can be carried out by methods including, but not limited to, Atom Transfer Radical Polymerization (ATRP), nitroxide-mediated living free radical polymerization (NMP), ring-opening polymerization (ROP), degenerative transfer (DT), or Reversible Addition Fragmentation Transfer (RAFT). In specific embodiments, a polymer can be a prepared by controlled (living) radical polymerization, such as reversible addition-fragmentation chain transfer (RAFT) polymerization. Such methods and approaches are generally known in the art. Alternatively, a polymer can be a prepared by conventional polymerization approaches, including conventional radical polymerization approaches.

In some embodiments, a polymer is prepared by a method other than by stepwise coupling approaches involving a sequence of multiple individual reactions (e.g., such as known in the art for peptide synthesis or for oligonucleotide synthesis).

In some embodiments, polymers prepared by controlled radical polymerization, such as reversible addition-fragmentation chain transfer (RAFT) polymerization, include moieties other than the monomeric residues (repeat units). For example, and without limitation, such polymers can include polymerization-process-dependent moieties at the α-end or at the ω-end of the polymer chain Typically, for example, a polymer chain derived from controlled radical polymerization such as RAFT polymerization may further comprise a radical source residue covalently coupled with the α-end thereof. For example, the radical source residue can be an initiator residue, or the radical source residue can be a leaving group of a reversible addition-fragmentation chain transfer (RAFT) agent. Typically, as another example, a polymer derived from controlled radical polymerization such as RAFT polymerization may further comprise a chain transfer residue covalently coupled with the ω-end thereof. For example, a chain transfer residue can be a thiocarbonylthio moiety having a formula —SC(═S)Z, where Z is an activating group. Typical RAFT chain transfer residues are derived from radical polymerization in the presence of a chain transfer agent selected from xanthates, dithiocarbamates, dithioesters, trithiocarbonates, and pyrazole carbodithioates. The process-related moieties at α-end or at the ω-end of the polymer or between blocks of different polymers can comprise or can be derivatized to comprise functional groups, e.g., suitable for covalent linking, etc.

In various embodiments, any monomer suitable for providing the polymers described herein can be used in the methods and compositions described herein. In some embodiments, monomers suitable for use in the preparation of polymers provided herein include, by way of non-limiting example, one or more of the following monomers: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysillpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-arylmaleimide, N-phenylmaleimide, N-alkylmaleimide, N-butylimaleimide, N-vinylpyrrolidone, N-vinylcarbazole, butadiene, isoprene, chloroprene, ethylene, propylene, 1,5-hexadienes, 1,4-hexadienes, 1,3-butadienes, 1,4-pentadienes, vinylalcohol, vinylamine, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, vinylnaphthalene, vinyl pyridine, ethylvinylbenzene, aminostyrene, vinylimidazole, vinylpyridine, vinylbiphenyl, vinylanisole, vinylimidazolyl, vinylpyridinyl, vinylpolyethyleneglycol, dimethylaminomethylstyrene, trimethylammonium ethyl methacrylate, trimethylammonium ethyl acrylate, dimethylamino propylacrylamide, trimethylammonium ethylacrylate, trimethylanunonium ethyl methacrylate, trimethylammonium propyl acrylamide, dodecyl acrylate, octadecyl acrylate, or octadecyl methacrylate monomers, or combinations thereof.

Monomers modified to comprise drug, ligand or stabilizing moieties are useful for generating co-polymers that comprise the drug, ligand and an optional stabilizing moiety. Further details regarding drug copolymer compositions useful in the methods and compositions described herein are provided below.

### Copolymers

In one embodiment, the compositions described herein provide a copolymer comprising a first constitutional unit having a pendant group comprising a therapeutic agent covalently coupled to the copolymer by a cleavable linkage. In other embodiments, the drug copolymer compositions comprise a second constitutional unit having a copolymer-stabilizing pendant group selected from the group consisting of a poly(ethylene oxide) group and a zwitterionic group.

In one embodiment, the compositions described herein provide drug copolymer compositions including a copolymer comprising:
(a) a first constitutional unit having a pendant group comprising a therapeutic agent covalently coupled to the copolymer by a cleavable linkage;
(b) a second constitutional unit having a pendant anionic group; and
(c) a third constitutional group having a pendant cationic group.

In certain embodiments of the drug copolymer compositions, the cleavable linkage is cleavable by hydrolysis. Representative cleavable linkages include ester, acetal, hemiacetal, hemiacetal ester, disulfide, hydrazide groups and self-immolating linkages. In certain embodiments, the cleavable linkage is an aliphatic ester (e.g., —CH₂—C(═O)—O—). In other embodiments, the cleavable linkage is a phenyl ester (e.g., —C₆H₄—C(═O)—O—).

In some embodiments, the cleavable linkage is a self-immolating linkage. In some embodiments, the cleavable linkage comprises a self-immolating linkage comprising a structure selected from

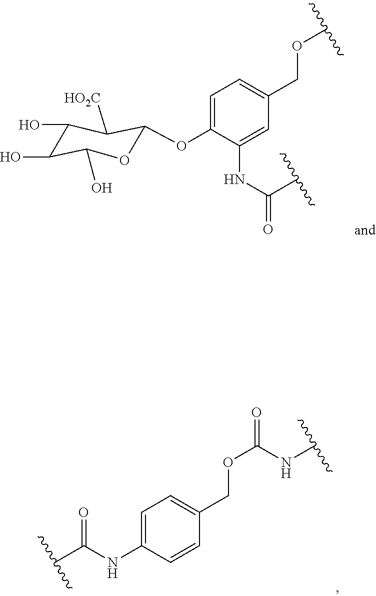 [SMILES: *OCc1ccc(OC2OC(O)C(O)C(O)C2C(=O)O)c(NC(*)=O)c1 ; *NC(=O)OCc1ccc(NC(*)=O)cc1]

each of which can be optionally substituted. Other non-limiting examples of self-immolating groups are those disclosed in WO2005082023, EP1912671, and WO2015038426; the relevant disclosure of each document is incorporated herein by reference.

In other embodiments, the cleavable linkage is a self-immolating linkage comprising a structure selected from the following:

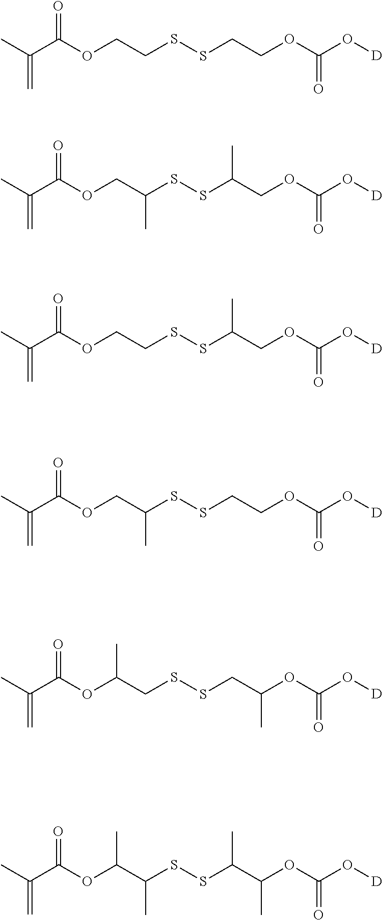

For further guidance regarding self-immolating linkages for use with the methods and compositions described herein, one of skill in the art can refer to e.g., Danial et al. (2016) “Combination anti-HIV therapy via tandem release of prodrugs from macromolecular carriers” Polym Chem 7:7477-7487, which is herein incorporated by reference in its entirety.

In certain embodiments, the cleavable linkage is cleavable by enzymatic action. Representative cleavable linkages include amino acid sequences cleavable by enzymatic (e.g., peptidase) action including valine-citrulline-para-aminobenzoic acid, valine-alanine and phenylalanine-lysine.

In other embodiments, the cleavable linkage is cleaved by beta-glucuronidase.

The drug copolymer compositions useful in the compositions and methods described herein release therapeutic agents. In certain embodiments, the therapeutic agent is a small molecule therapeutic agent (i.e., having a molecular weight less than about 800 g/mole). In other embodiments, the therapeutic agent is a peptide therapeutic agent. Representative therapeutic agents releasable by the polymeric carriers are described below.

The drug copolymer compositions useful in the compositions and methods described herein have a high therapeutic agent density. For the poly(ethylene oxide) and zwitterionic group-containing copolymers described above, the ratio of the number of first constitutional units to the number of second constitutional units is from about 1:1 to about 1:2. For the polyampholyte containing copolymers described above, the ratio of the number of first constitutional units to the number of second and third constitutional units is from about 1:1 to about 1:2.

For the drug copolymer compositions that include poly(ethylene oxide) groups, the poly(ethylene oxide) group has at least five ethylene oxide repeating units (i.e., —(CH₂CH₂O)ₙ—, where n ≥5). In certain embodiments, the poly(ethylene oxide) group has from five (5) to thirty (30) ethylene oxide repeating units (i.e., —(CH₂CH₂O)ₙ—, where n=5-30).

For the drug copolymer compositions that include zwitterionic groups, representative zwitterionic groups include carboxybetaine groups, sulfobetaine groups, and phosphobetaine groups.

For the drug copolymer compositions that include ampholyte groups, the carriers include anionic groups (negatively charged groups) that include an oxyanion (e.g., —CO₂⁻, —SO₃⁻) or an oxygen-containing acid group that becomes deprotonated under physiological conditions (e.g., CO₂H), and include cationic groups (positively charge groups) that include a nitrogen-containing group that becomes protonated under physiological conditions (e.g., primary, secondary, or tertiary amine) or a nitrogen-containing group having a permanent positive charge. For the drug copolymer compositions that include ampholyte groups, the number of second and third constitutional units may be substantially the same.

For the copolymers described above, in certain embodiments the copolymer is a random copolymer and, in other embodiments, the copolymer is a block copolymer (e.g., a diblock copolymer or a triblock copolymer). In certain embodiments, when the copolymer is a poly(ethylene oxide) or zwitterionic containing block copolymer, the block copolymer has a first block comprising the first constitutional unit comprising the therapeutic agent and a second block comprising the second constitutional unit comprising the copolymer-stabilizing pendant group. In other embodiments, when the copolymer is an ampholyte containing block copolymer, the diblock copolymer has a first block comprising the first constitutional unit comprising the therapeutic agent covalently attached to the first block (e.g., via a pendant group), and having a second block comprising the second and third constitutional units comprising the anionic and cationic groups, respectively.

In some embodiments, the drug copolymer compositions as described herein comprise at least one ligand that specifically binds a heterologous ligand-binding polypeptide. In certain instances, the ligand is incorporated into the drug copolymer compositions by copolymerization of a ligand monomer with a suitable stabilizing group monomer and a suitable polymerizable prodrug monomer. As used herein, a prodrug monomer is a molecule comprising a polymerizable group covalently linked to a drug moiety via a cleavable linking moiety in such manner that the drug can be released upon cleavage of the linking group. In other instances, the ligand is introduced into the copolymer by virtue of performing the copolymerization of a suitable stabilizing group monomer and a suitable polymerizable prodrug monomer in the presence of a ligand-comprising chain transfer group. Suitable ligands include small molecules that are one of a binding pair (e.g., ligand is an antigen and ligand binding partner is an antibody or functional fragment thereof). Representative ligands include (i) fluorescent proteins (e.g., fluorescein, rhodamine, etc.), (ii) affinity ligands (e.g., biotin or biotin acceptor domain (e.g., GLNDIFEAQKIEWHE), 9-cis retinoic acid, 8-aryl hydrocarbon, or sialic acid), (iii) peptide tags, such as polyhistidine (HHHHHH), c-Myc (EQKLISEEDL), human influenza agglutinin (HA) (YPYDVPDYA), FLAG (DYKDDDDK), thrombin fragment (LVPRGS), V5 (GKPIPNPLLGLDST), SB1 (PRPSNKRLQQ), Protein C fragment (EDQVDPRLIDGK), SV40 nuclear localization signal (PKKKRKVG), VSVG (YTDIEMNRLGK), Factor Xa (IDGR), or T7 (MASMTGGQQMG), (iv) small proteins, such as calmodulin binding protein, and (v) small molecule binders of intracellular proteins, such as CBP bromodomain ligands (see e.g., J Am Chem Soc (2014) 136(26), pg 9308-9319; DOI:10.1021/ja412434f).

Representative monomers suitable for the preparation of drug copolymers useful in the methods and compositions described herein are described in detail below.

The drug copolymer compositions include the following random copolymers. In certain embodiments, the drug copolymer composition comprises a random copolymer having the formula (I):

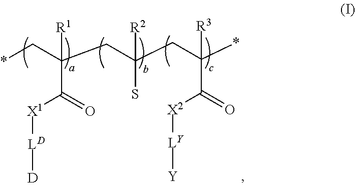

wherein
R¹, R², and R³ are independently selected from hydrogen and methyl,
S is a copolymer-stabilizing group,
X¹ and X² are independently 0 or NH,
D is a therapeutic agent or therapeutic agent residue,
Y is a ligand
L^{D} is a linking group comprising one or more cleavable linkages,
L^{Y} is a linking group optionally comprising one or more cleavable linkages,
a is an integer from about 5 to about 500,
b is an integer from about 5 to about 500,
c is an integer from 1 to about 500, and
each * represents the copolymer terminus.

In other embodiments, the drug copolymer composition comprises a random copolymer having the formula (II):

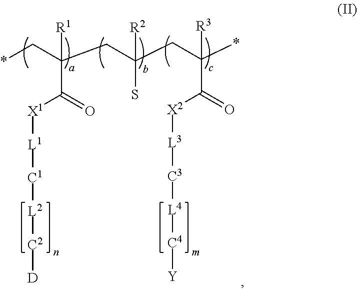

wherein
R¹, R², and R³ are independently selected from hydrogen and methyl,
S is a copolymer-stabilizing group,
X¹ and X² are independently 0 or NH,
D is a therapeutic agent or therapeutic agent residue,
Y is a ligand,
C¹ is a cleavable linkage,
L¹ is a linking group that covalently couples C¹ to X¹,
C² at each occurrence is an independent cleavable linkage,
L² is a linking group that covalently couples C¹ to C²,
C³ is a cleavable linkage,
L³ is a linking group that covalently couples C³ to X²,
C⁴ at each occurrence is an independent cleavable linkage,
L⁴ is a linking group that covalently couples C³ to C⁴,
n and m are independently 0, 1, 2 or 3,
a is an integer from about 5 to about 500,
b is an integer from about 5 to about 500,
c is an integer from 1 to about 500, and
each * represents the copolymer terminus.

In some embodiments, the drug copolymer composition comprises a random copolymer having the formula (III):

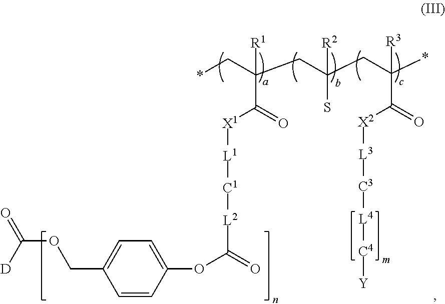

wherein
R¹, R², and R³ are independently H or CH₃,
S is a copolymer-stabilizing group,
and X² are independently O or NH,
D is a therapeutic agent or therapeutic agent residue,
Y is a ligand,
C¹ is a cleavable linkage,
L¹ is a linking group that covalently couples C¹ to X¹,
L² is a linking group,
C³ is a cleavable linkage,
L³ is a linking group that covalently couples C³ to X²,
C⁴ at each occurrence is an independent cleavable linkage,
L⁴ is a linking group that covalently couples C³ to C⁴,
n is 0 or 1,
m is 0, 1, 2 or 3,
a is an integer from about 5 to about 500,
b is an integer from about 5 to about 500,
c is an integer from 1 to about 500, and
each * represents the copolymer terminus.

Representative embodiments of drug copolymer compositions comprising copolymers of formulae (I)-(III) are described below.

In certain embodiments for copolymers of formulae (I)-(III), a is an integer from about 5 to about 500, b is an integer from about 5 to about 500, and c is an integer from 1 to about 500.

In certain embodiments, X¹ is O. In other embodiments, X² is O.

In certain embodiments, L¹ is a linker group comprising a carbon chain having from two to ten carbon atoms and optionally from two to four oxygen or nitrogen atoms. In certain embodiments, L¹ is —(CH₂)ₙ—, where n is 2-10. In other embodiments, L¹ is —(CH₂CH₂O)ₙ—, where n is 2-4.

In certain embodiments, L² is a linker group comprising a carbon chain having from two to ten carbon atoms and optionally from two to four oxygen or nitrogen atoms. In other embodiments, L² is —(CH₂)ₙ— where n is 2-10. In further embodiments, L² is —(CH₂CH₂O)ₙ— where n is 2-4.

C¹ and C² are functional groups cleavable by hydrolysis or enzymatic action. In certain embodiments, C¹ and C² are independently selected from ester, acetal, hemiacetal, hemiacetal ester, disulfide, self-immolating linkages, and hydrazine groups. In certain embodiments, C¹ and C² are independently selected from aliphatic ester (e.g., —CH₂—C(═O)—O—) and phenyl ester (e.g., —C₆H₄—C(═O)—O—) groups. For phenyl ester linkages, it will be appreciated that the phenyl group can be substituted with one, two, three, or four groups to adjust the rate of phenyl ester cleavage. In general, the electron withdrawing groups increase the rate of cleavage and electron donating groups decrease the rate of cleavage. Representative phenyl group substituents include C1-C6 alkyl groups (e.g., methyl, ethyl), C1-C6 alkoxy groups (e.g., methoxy, ethoxy), halo groups (e.g., fluoro, chloro, bromo), carbonyl containing groups (e.g., —C(═O)—CH₃, —C(═O)—OCH₃, —C(═O)—NH₂). In certain embodiments, the cleavable linkage comprises an amino acid sequence cleavable by enzymatic action. Representative cleavable linkages include amino acid sequences cleavable by enzymatic (e.g., peptidase) action including valine-citrulline-para-aminobenzoic acid, valine-alanine and phenylalanine-lysine.

In other embodiments, the cleavable linkage is cleaved by beta-glucuronidase.

In some embodiments, C¹ and/or C² are stable in the circulation system and are cleavable under physiological conditions at the target site.

In certain embodiments, L³ is a linker group comprising a carbon chain having from two to ten carbon atoms and optionally from two to four oxygen or nitrogen atoms. In certain embodiments, L³ is —(CH₂)ₙ—, where n is 2-10. In other embodiments, L¹ is —(CH₂CH₂O)ₙ—, where n is 2-4.

In certain embodiments, L⁴ is a linker group comprising a carbon chain having from two to ten carbon atoms and optionally from two to four oxygen or nitrogen atoms. In other embodiments, L⁴ is —(CH₂)ₙ— where n is 2-10. In further embodiments, L⁴ is —(CH₂CH₂O)ₙ— where n is 2-4.

C³ and C⁴ are functional groups cleavable by hydrolysis or enzymatic action. In certain embodiments, C³ and C⁴ are independently selected from ester, acetal, hemiacetal, hemiacetal ester, disulfide, and hydrazide groups as well as self-immolating linkages. In certain embodiments, C³ and C⁴ are independently selected from aliphatic ester and phenyl ester groups.

As noted above, the drug copolymer compositions as described herein release therapeutic agents. In certain embodiments, the therapeutic agent is a small molecule therapeutic agent (i.e., having a molecular weight less than about 800 g/mole). In other embodiments, the therapeutic agent is a peptide therapeutic agent. Representative therapeutic agents releasable by the polymeric carriers, as disclosed herein, are described below.

The drug copolymer compositions have a high therapeutic agent density. For the polymers of formulae (I)-(III), in certain embodiments, a:b is from about 2:1 to about 1:2. In certain embodiments, a:b is from about 2:1 to about 1:1. In other embodiments, a:b is about 1:1.

For certain embodiments of the drug copolymer compositions of formulae (I)-(III) where S is a poly(ethylene oxide), the drug monomer and hydrophilic monomer are less than about 200 constitutional units total (a+b≤200), and in other embodiments, about 15-30 units total (a+b=15-30). For certain embodiments of the polymers of formulae (I)-(III) where S is a zwitterionic group, the drug monomer and hydrophilic monomer is less than about 400 units total (a+b≤400), and in other embodiments, about 25-50 units total (a+b=25-50). Optimum control over the polymerization is observed in these ranges.

Alternatively, as described herein, the copolymer comprises a drug-containing block with an additional stabilizing polymer block for stabilization in aqueous solution (i.e., copolymer-stabilizing group). In this embodiment, the drug-containing block can include higher relative amounts of the drug-containing constitutional unit (e.g., from about 50 to approaching 100 mole or weight % of the block) than the other units. As described herein, the drug-containing block can include additional constitutional units to impart desirable properties (e.g., to modulate the drug release rate). In certain embodiments, the drug-containing block includes 100 mole or weight % drug-containing constitutional units.

In one embodiment, two or more discrete copolymers that release one or more drugs at different rates can be delivered by a single cell. This has the advantage of, for example, providing rapid initial release (e.g., a bolus dose) to establish an effective level of drug in a given cellular or tissue microenvironment, combined with slower, sustained release of a drug over time in the same locale. In one embodiment, the drug is the same, and different discrete copolymer compositions or constitutional units that release the same drug at different rates are bound to the surface of the same cell via either the same or different heterologous ligand-binding polypeptides on the cell. In another embodiment, two or more different drugs are released from the discrete drug copolymer compositions on the same cell. Co-polymer drug release kinetics can be tailored by one of ordinary skill in the art using known principles.

When it is desirable for the drug copolymer composition as described herein to serve as a therapeutic agent depot, the drug-containing block can include significantly greater amounts of the drug (e.g., from about 50 to approaching 100 mole or weight % of the block). In certain embodiments, these blocks can include from 50-99, 50-95, 50-90, 50-80, 50-70 mole or weight percent of drug-containing constitutional unit.

In certain embodiments, for the polymeric carriers of formulae (I)-(III), the copolymer-stabilizing group S comprises a poly(ethylene oxide) group. In certain embodiments, S comprises a poly(ethylene oxide) group having at least five ethylene oxide repeating units (i.e., —(CH₂CH₂O)ₙ—, where n≥5). In certain embodiments, S comprises a poly(ethylene oxide) group having from five (5) to thirty (30) ethylene oxide repeating units (i.e., —(CH₂CH₂O)ₙ—, where n=5-30). In certain embodiments, S is

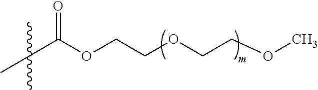

wherein m is an integer from 5 to 30.

In some embodiments, S comprises a poly(ethylene oxide) group having a molecular weight of 1000 Daltons or more (e.g., 2000 Da or more, 3000 Da or more, 4000 or more, 5000 or more, or 7000 or more) and/or 10 kDa or less (e.g., 7000 Da or less, 5000 Da or less, 4000 Da or less, 3000 Da or less, or 2000 Da or less).

In certain embodiments, for the polymers of formulae (I)-(II), copolymer-stabilizing group S comprises a zwitterionic group. In certain embodiments, S comprises a zwitterionic group selected from the group consisting of a carboxybetaine group, a sulfobetaine group, and a phosphobetaine group. In certain embodiments, S is selected from

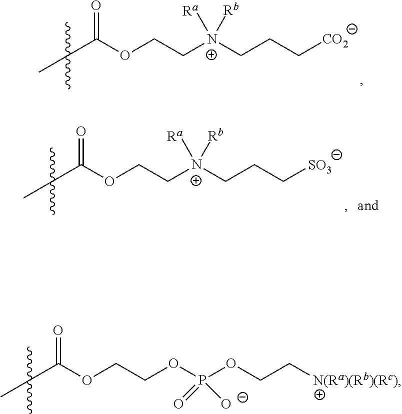

wherein R^{a}, R^{b}, and R^{c} are independently selected from hydrogen and C1-C6 alkyl.

In some aspects, provided herein are methods for making the drug copolymer compositions as described herein. As noted above and described herein, the drug copolymer compositions as described herein are prepared by copolymerization of a polymerizable prodrug monomers and monomers that include stabilizing groups containing monomer (e.g., by a controlled polymerization such as RAFT polymerization). The polymerization process can be one that provides a random copolymer or a diblock copolymer. The copolymer can be further subject to chain extension to provide a triblock copolymer from a diblock, or a star, branched, or dendrimer-like, higher order copolymer. Chain extension can be carried out to with suitable monomers or comonomers to provide blocks such as endosomolytic blocks, or hydrophobic blocks, that include the therapeutic agent to be released.

In some embodiments, the controlled polymerization is a RAFT polymerization. In certain embodiments, the RAFT polymerization is performed with a chain transfer reagent that is a xanthate, dithiocarbamate, dithioester, trithiocarbonate or a pyrazole carbodithioate.

In certain embodiments, the chain transfer reagent is of the formula (IV):

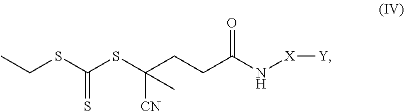

wherein X is a linking group, and Y is a ligand that specifically binds a heterologous ligand-binding polypeptide.

In certain exemplary embodiments, a representative chain transfer reagent comprising a ligand has the structure of formula (V):

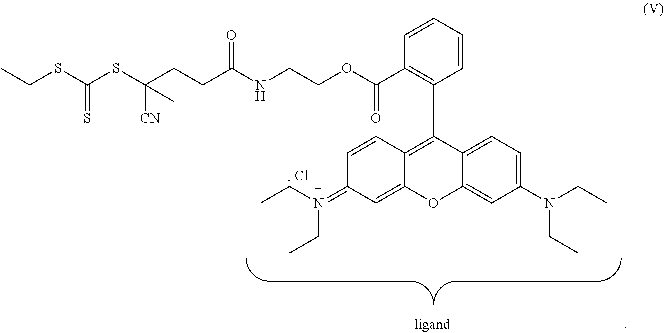

### Therapeutic Agents

The drug copolymer composition useful in the methods and compositions described herein is a macromolecular prodrug that releases one or more therapeutic agents. The therapeutic agent can be one or more of many different types of therapeutic agent (e.g., an antibiotic agent, an antimalarial agent, an anti-viral agent, a chemotherapeutic agent, a kinase inhibitor, an immunomodulator, etc.).

In some embodiments, the therapeutic agent is modified in such a way that hydrolysis or enzymatic cleavage provides the parent/active therapeutic agent (with reference to formulae (I)-(III), D is a therapeutic agent). In some embodiments, the therapeutic agent is a small molecule comprising a functional group (e.g., OH, SH, COOH, NH₂, or NHR) that can be covalently linked to the monomer via a cleavable linkage. In some embodiments, cleavage from the polymer does not provide the original (i.e., parent/active) therapeutic agent, but rather releases a modified therapeutic agent, sometimes referred to in the art as a prodrug, that can undergo further modification in a physiological environment such that the modified therapeutic agent can then release the active therapeutic agent in an active form at a different rate than the initial cleavage rate (with reference to formulae (I)-(III), D is a therapeutic agent residue). In some embodiments, even though an active therapeutic agent has been modified to provide a polymerizable prodrug monomer, release of the modified therapeutic agent can still provide a therapeutically active molecule and have the desired therapeutic activity.

In some embodiments, the therapeutic agent is selected from an antibiotic agent, an anti-viral, a kinase inhibitor, a growth factor receptor inhibitor, a chemotherapeutic, an estrogen receptor (ER) ligand, a Toll-Like Receptor (TLR) antagonist, an indoleamide 2,3dioxygenase inhibitor, a TGFβ receptor I (TβRI) inhibitor, an oligonucleotide therapeutic agent, and a cyclic dinucleotides (CDNs) STING agonist.

Examples of antibiotic agents include amikacin, gentamicin, neomycin, netilmicin, tobramycin, paromomycin, streptomycin, spectinomycin, geldanamycin, herbimycin, rifaximin, loracarbef, mertapenem, doripenem, imipenem, meropenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriazxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin methicillin, nafcillin, oxicillin, penicillin, piperacillin, temocillin, ticarcillin, piperacillin, ticarcillin, bacitracin, colistin, polymyxin B, xacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilamide, sulfasalazine, sulfisoxazole, trimethoprim-sulfamethoxazole, sulfonamidochrysoidine, demeclocycline, doxycycline, minocycline, oxytetracycline, tetracycline, clofazimine, dapsone, capreomycin, cycloserine, ethambutol, ethionamide, isoniazid, pyrazinamide, rifampin, rifabutin, rifapentine, streptomycin, arsphenamine, chloramphenicol, fosfomycin, fusidic acid, metronidazole, mupirocin, platensimycin, quinupristin, thiamphenicol, tigecycline, tinidazole, and trimethoprim. In some embodiments, the antibiotic agent is ciprofloxacin, meropenem, doxycycline, and/or ceftazidime.

Examples of kinase inhibitors include, for example, afatinib, axitinib, bevacizumab, bosutinib, cetuximab, crizotinib, dasatinib, erlotinib, fostamatinib, gefitinib, ibrutinib, imatinib, lapatinib, lenvatinib, nilotinib, panitumumab, pazopanib, pegaptanib, ranibizumab, roxolitinib, sorafenib, sunitinib, SU6656, trastuzumab, tofacitinib, and vemurafenib. In some embodiments, the kinase inhibitor is dasatinib.

In some embodiments, the therapeutic agent is an estrogen receptor (ER) ligand. Examples of an ER ligand are 4-hydroxytamoxifen, CMP8 (9a-(4-Chlorobenzyl)-7-hydroxy-4-[4-(2-piperidin-1-ylethoxy)phenyl]-1,2,9,9a-tetrahydro-3H-fluoren-3-one), fulvestrant, and raloxifene.

In some embodiments, the therapeutic agent is a chemotherapeutic agent, such as a vinca alkaloid or a taxane. Examples of chemotherapeutic agents include illudin, aminitin, gemcitabine, etoposide, docetaxel, camptothecin, and paclitaxel.

In some embodiments, the drug copolymer compositions of the present invention comprise a single therapeutic agent. In other embodiments, the drug copolymer compositions are combination-therapy compositions comprising two or more different therapeutic agents. In one embodiment, such combination-therapy drug copolymer compositions comprise a potent TGFβ receptor I (TβRI) inhibitor (e.g., Galunisertib (LY2157299)) and a Toll-Like-Receptor (TLR) antagonist (e.g., resiquimod).

In some embodiments, the therapeutic agent is an immunomodulator, such as a corticosteroid (e.g., betamethasone, budesonide, cortisone, dexamethasone, hydrocortisone, methylprednisolone, prednisolone, and/or prednisone) and can be used in the treatment of e.g., autoimmune disease.

In some embodiments, the therapeutic agent is an immunomodulator useful in the treatment of e.g., allergy. Such immunomodulators include, but are not limited to, STAT-6 inhibitors, AP-1 inhibitors, and NFκB inhibitors.

In some embodiments, the therapeutic agent is an immunomodulator useful in the treatment of e.g., chronic inflammation. Such immunomodulators include, but are not limited to, azathioprine or aminosalicylates e.g., mesalamine (PENTASA™, APRISO™, ASACOL™)

In some embodiments, the therapeutic agent is an anti-viral agent or an anti-retroviral agent. Non-limiting examples of anti-viral agents include e.g., abacavir (ZIAGEN™), acyclovir (ZOVIRAX™) adefovir (HEPSERA™), amantadine (SYMMETREL™), amprenavir (AGENERASE™), rintatolimod (AMPLIGEN™), umifenovir (ARBIDOL™), atazanavir (REYATAZ™), ATRIPLA™ (Efavirenz/emtricitabine/tenofovir), balvir, cidofovir (VISTIDE™), COMBIVIR™ (amivudine/zidovudine), dolutegravir (TIVICAY™), darunavir (PREZISTA™), delavirdine (RESCRIPTOR™), didanosine (VIDEX™), docosanol (ABREVA™), edoxudine, efavirenz (SUSTIVA™), emtricitabine (EMTRIVA™), enfuvirtide (FUZEON™), entecavir (BARACLUDE™) ecoliever, famciclovir (FAMVIR™), fomivirsen (VITRAVENE™), fosamprenavir (LEXIVA™ TELZIR™), foscarnet (FOSCAVIR™), fosfonet, fusion inhibitors (anti-retroviral, e.g., maraviroc (SELZENTRY™, CELSENTRI™), enfuvirtide (FUZEON™)) ganciclovir (CYTOVENE™, CYMEVENE™, VITRASERT™), ibacitabine, inosine pranobex (IMUNOVIR™), idoxuridine, imiquimod (ALDARA™), indinavir (CRIXIVAN™), inosine, integrase strand transfer inhibitors (anti-retrovirals; dolutegravir (TIVICAY™), elvitegravir (VITEKTA™), raltegravir (ISENTRESS™), BI 224436, bictegravir, cabotegravir, MK-2048), interferon type III, interferon type II, interferon type I, interferon, iamivudine (EPIVIR™), lopinavir, loviride, maraviroc (SELZENTRY™, CELSENTRI™), moroxydine, methisazone, nelfinavir (VIRACEPT™), nevirapine (VIRAMUNE™), nexavir, nitazoxanide (ALINIA™, NIZONIDE™), nucleoside analogues, novir, oseltamivir (TAMIFLU™), peginterferon alfa-2a (PEGASYS™), penciclovir (DENAVIR™), peramivir (RAPIVAB™), pleconaril, podophyllotoxin (PODOFILOX™), raltegravir (ISENTRESS™), a reverse transcriptase inhibitor, ribavirin (TRIBAVIRIN™), rimantadine (FLUMADINE™), ritonavir (NORVIR™), pyramidine, saquinavir (INVIRASE™, FORTOVASE™), sofosbuvir (SOVALDI™), stavudine (ZERIT™), synergistic enhancer (antiretroviral), telaprevir (INCIVEK™, INCIVO™), tenofovir, tenofovir disoproxil (VIREAD™), tipranavir (APTIVUS™), trifluridine (VIROPTIC™, LONSURF™), TRIZIVIRA™ (abacavir/lamivudine/zidovudine), tromantadine (VIRU-MERZ™), TRUVADA™ (emtricitabine/tenofovir), valaciclovir (VALTREX™), valganciclovi (VALCYTE™), vicriviroc, vidarabine, viramidine, dideoxycytosine (zalcitabine, HMD™), zanamivir (RELENZA™), clevudine, telbivdine or zidovudine (anti-retroviral, RETROVIR™).

In some embodiments, the drug copolymer compositions as described herein comprise a single therapeutic agent. In other embodiments, the drug copolymer compositions are combination-therapy compositions comprising two or more different therapeutic agents. In one embodiment, such combination-therapy drug copolymer compositions comprise a potent TGFβ receptor I (TβRI) inhibitor (e.g., Galunisertib (LY2157299)) and a Toll-Like-Receptor (TLR) antagonist (e.g., resiquimod). In some other embodiments such combination-therapy drug copolymer compositions comprise a lamivudine and zidovudine.

### Prodrug Monomers

The methods and compositions described herein provide prodrug monomers and related copolymers. In certain embodiments, the prodrug monomers include poly(ethylene glycol) constitutional units.

In some embodiments, the polymer comprises one or more residues derived from a monomer of formula (VI):

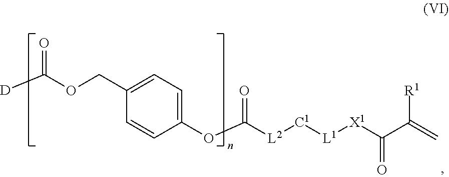

wherein:

R¹ is H or CH₃;

X¹ is O or NH; C¹ is a group cleavable by hydrolysis or enzymatic action;

L1 and L² are linking groups;

n is 0 or 1;

D is a therapeutic agent or therapeutic agent prodrug.

In some embodiments of formula (VI), L¹ comprises a carbon chain having from two to ten carbon atoms and optionally from two to four oxygen or nitrogen atoms. In certain embodiments, L1 is —(CH₂)ₙ—, wherein n is 2-10. In other embodiments, L1 is —(CH₂CH₂O)ₙ—, wherein n is 1-30.

In some embodiments of formula (VI), L² comprises a carbon chain having from two to ten carbon atoms and optionally from two to four oxygen or nitrogen atoms. In certain embodiments, L² is —(CH₂)ₙ—, wherein n is 2-10.

In certain embodiments of formula (VI), C¹ is a functional group selected from ester, acetal, hemiacetal, hemiacetal ester, and hydrazide groups. In other embodiments, C¹ is an ester (—C(O)O—) group.

In certain instances of formula (VI), R₁ is CH₃. In other instances, X¹ is O.

In some embodiments of formula (VI), the monomer has a structure of formula (VII):

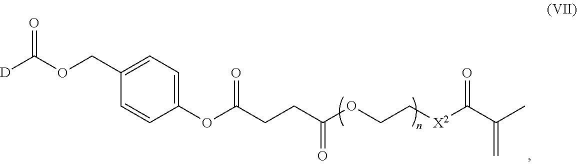

wherein n is 1-30, X² is NH or O, and D is a therapeutic agent or therapeutic agent residue.

In certain embodiments, the polymer comprises one or more residues derived from a monomer of formula (VIII):

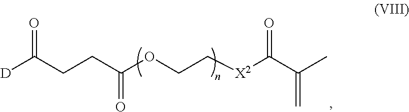

wherein n is 1-30, X² is NH or O, and D is a therapeutic agent or therapeutic agent residue.

In some embodiments of formulae (VII) or (VIII), X² is O and n is 1.

In some embodiments, the polymer comprises one or more residues derived from a monomer selected from those illustrated in the Figures and/or working Examples provided herein.

### Representative Prodrug Monomers

The preparation and properties of representative ciprofloxacin prodrug monomers and related copolymers having poly(ethylene glycol) constitutional units are described in the working Examples. The representative ciprofloxacin prodrug monomers and related copolymers have cleavable linkers (aliphatic ester and phenolic ester groups) that efficiently release ciprofloxacin at therapeutically effective rates.

### Polymer Definitions

The following definitions relate to polymers in general and are useful in understanding the copolymers described herein.

The term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

The term “repeat unit” corresponds to the smallest constitutional unit, the repetition of which constitutes a regular macromolecule (or oligomer molecule or block).

The term “end group” (in certain embodiments, * in formulae (I) and (II)) refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

A “monomer” is a polymerizable compound that, on polymerization, contributes one or more constitutional units in the structure of the polymer.

The term “polymer” refers to the product that is the result of polymerization of a single monomer or several monomers, some of which may be the same or different.

The term “homopolymer” refers to the product of polymerization of the same monomer.

The term “copolymer” refers to a polymer that is the result of polymerization of two or more different monomers. The number and the nature of each constitutional unit can be separately controlled in a copolymer. The constitutional units can be disposed in a purely random, an alternating random, a regular alternating, a regular block, or a random block configuration unless expressly stated to be otherwise. A purely random configuration can, for example, be: x-x-y-z-x-y-y-z-y-z-z-z . . . or y-z-x-y-z-y-z-x-x . . . . An alternating random configuration can be: x-y-x-z-y-x-y-z-y-x-z . . . , and a regular alternating configuration can be: x-y-z-x-y-z-x-y-z . . . .

The term “block copolymer” refers to a polymer formed of two or more covalently joined blocks of shorter homopolymers, i.e., a structure in which distinct sub-combinations of constitutional units are joined together. In certain instances, a “block” refers to a segment or portion of a homopolymer having a particular characteristic (e.g., a hydrophilic segment) or a certain composition distinct from the composition of the other blocks of the polymer.

An exemplary diblock copolymer is a polymer that comprises two blocks. A schematic generalization of such a diblock copolymer can look like: [AₐB_{b}C_{c} . . . ]ₘ—[XₓY_{y}Z_{z} . . . ]ₙ, wherein each capital letter stands for a constitutional unit, each subscript to a constitutional unit represents the mole fraction of that unit in the particular block, the three dots indicate that there may be more (or there may also be fewer) constitutional units in each block, and m and n indicate the molecular weight of each block in the diblock copolymer. As suggested by the schematic, the number and the nature of each constitutional unit are separately controlled for each block. It is understood that the schematic is not meant and should not be construed to infer any relationship whatsoever between the number of constitutional units or the number of different types of constitutional units in each of the blocks. Nor is the schematic meant to describe any particular arrangement of the constitutional units within a particular block.

Representative embodiments describing prodrug monomers and related copolymers prepared from the monomers are provided in the Examples herein below.

### Cells for Drug Delivery

Methods and compositions described herein rely, in part, upon cells to deliver drugs or therapeutic agents. For therapeutic purposes, the cells can be any appropriate mammalian cell, but are preferably of the same species as the subject to be treated. In some embodiments, the cell is allogeneic to the subject to be treated; in others, the cell is autologous to the subject.

Cells for drug delivery as described herein are modified to express a heterologous ligand-binding polypeptide that specifically binds a ligand attached to or incorporated into a drug copolymer composition. Heterologous ligand binding polypeptides are described further herein below. As noted, any mammalian cell type that can be transduced to express a heterologous ligand-binding polypeptide that permits loading with a ligand:drug copolymer composition can be adapted to deliver a drug or therapeutic agent from a drug copolymer. Examples include fibroblasts, T cells, macrophages, epithelial cells (including but not limited to fibroblasts, T cells, Natural Killer cells, and monocytes), monocytic derived cell populations (including but not limited to macrophages and dendritic cells), osteoclasts, secretory endothelial cells, hematopoietic stem cells, B cells, among others.

An important class of cells useful in the methods and compositions described herein is immune cells, which can have not only the capacity to traffic to or be directed to a given location or microenvironment, e.g., a tumor microenvironment or a site of infection or inflammation, but also the ability to directly affect a target cell or to influence the activities of one or more types of cell in such microenvironment. Various immune cells can have direct effector functions and/or can produce and release cytokines that, for example, recruit or influence other effector cells. The following discusses general considerations for, and cells useful in immunotherapy as applicable to the methods and compositions described herein. T cells and other immune cell types, such as macrophages, which have intrinsic effector functions, have advantages for the treatment of, for example, cancer and infection (e.g., chronic infection) using the approaches described herein. The following description accordingly refers to the isolation and modification of T lymphocytes. However, it should be understood that the compositions and methods described can be adapted and applied to any of a variety of different cell types. That is, the description as centered on T cells is illustrative, and the methods described should not be viewed as limited to use in immune cells generally or T cells specifically.

A mammalian immune system uses two general mechanisms to actively protect the body against invading environmental pathogens. One is a non-specific (or innate) inflammatory response. The other is a specific, acquired (or adaptive) immune response. Innate responses are fundamentally the same for each insult or injury while each adaptive response is custom tailored to a specific pathogen. Each adaptive response increases in intensity with each subsequent exposure, which is why they are called specific and adaptive responses.

Adaptive immunity is mediated by B- and T-lymphocytes, which are classes of specialized immune cells. The ability of subpopulations of B- and T-lymphocytes to recognize and respond against antigens expressed by pathogens accounts for the specificity of adaptive immune responses. Additionally, B- and T-lymphocytes are able to replicate themselves upon exposure to antigens. This ability of the B- and T-lymphocytes to replicate, following exposure to antigens accounts for an increase in intensity of the adaptive immune responses with repeated exposure to those antigens. Antigen-stimulated B- and T-lymphocytes are also very long-lived, which accounts for an adaptive immunologic memory.

B-lymphocytes produce, secrete, and mediate their functions through the actions of antibodies. B-lymphocyte-dependent immune responses are referred to as “humoral immunity” because antibodies are detected in body fluids (i.e., the humors), such as blood and secretions.

T-lymphocytes mediate their functions through the activities of effector T-lymphocytes. T-lymphocyte-dependent immune responses are referred to as “cell-mediated immunity” because cells, e.g., T-lymphocytes and macrophages, as opposed to antibodies, mediate effector activities of this arm of the immune system. The local actions of effector T-lymphocytes are amplified through synergistic interactions between effector T-lymphocytes and secondary effector cells, such as macrophages. Effector T-lymphocytes produce cytokines that activate macrophages to kill pathogens. Cytokines increase macrophages' ability to phagocytose and digest and/or kill pathogens. Cell-mediated immunity plays a major role in resistance to viruses, fungi, parasites, cancers, and bacteria that have the ability to live within cells of the innate immune system and sometimes also within other cells in the body.

A variety of medical interventions that augment the body's adaptive immune response(s) to pathogens have been developed. Medical interventions make use of the fact that acquired immune responses can be artificially manipulated. Those medical interventions are classified either as active or passive. Active immunological interventions may include, for example, exposing individuals to a weakened or inactivated pathogen that induces acquired immunity without causing disease and, additionally, protects the individual against later exposure to the same pathogen (e.g., vaccination or immunization).

Adaptive protective immunity can be passively transferred from one genetically identical individual to another, for example, in experimental model systems. Passive transfer has been used to establish that T-lymphocytes mediate viral immunity, immunity to obligate intracellular pathogens, and cancer immunity. T-lymphocytes transferred from an immune individual to a non-immune individual provide immune protection for the non-immune individual.

Passive transfer of immune T-lymphocytes between individuals can be accomplished in genetically identical animal models but is impractical as a medical intervention in humans unless the recipient is severely immune compromised because the genetic differences between individual humans would lead to T-lymphocytes being rapidly rejected by the recipient's immune system. That is, the recipient's immune system recognizes the donor T-lymphocytes as non-self, develops an immune response against them, and rapidly eliminates them from the body. However, passive transfer of T-lymphocytes in the same individual, (i.e., auto transplantation of T-lymphocytes or autologous adoptive transfer of native or modified T-lymphocytes) is feasible and safe as a medical intervention. Autotransplantation of bone marrow or peripheral blood containing T-lymphocytes as a source of stem cells following high dose chemotherapy or radiation is routinely performed as a medical intervention.

In some embodiments, the T cells are obtained from the subject to be treated. In other embodiments, the lymphocytes are obtained from allogeneic human donors, preferably healthy human donors. T lymphocytes can be collected in accordance with known techniques and enriched or depleted by known techniques such as affinity binding to antibodies in, e.g., flow cytometry and/or affinity selection.

Affinity selection refers to the selection of a specific molecule or cell having a selectable cell surface marker by binding to the molecule or marker or an epitope present thereupon with a binding affinity agent, which allows for one to select the desired molecule or cell of interest Affinity selection can be performed using, for example, antibodies, conjugated antibodies, lectins, aptamers, and/or peptides. The affinity agent can be immobilized, for example, on a solid support, e.g., a plastic or polycarbonate surface, plate, well, bead, particle or magnetic particle, among others. In some embodiments separation of a CD8+ population of T-cells and/or a CD4+ population of T-cells from a mixed population of T-cells is performed by affinity selection for T-cells having an epitope present on CD8 and/or CD4. In other embodiments, anti-CD8 or anti-CD4 antibodies or binding portions thereof are used to isolate or enrich a population comprising the cells of interest.

After enrichment and/or depletion steps, in vitro expansion of the desired T lymphocytes can be carried out in accordance with known techniques (see e.g., U.S. Pat. No. 6,040,177), or variations thereof that will be apparent to those skilled in the art. In some alternatives, the T cells are autologous T cells obtained from the patient. Preferably, the T cells are derived from thymocytes (naturally arising in humans); also specifically contemplated are those that are derived from engineered precursors, such as iPS cells.

For example, a desired T cell population or subpopulation can be expanded by adding an initial T lymphocyte population to a culture medium in vitro, and then adding to culture medium feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture for a time sufficient to expand the numbers of T cells. The non-dividing feeder cells can comprise gamma-irradiated PBMC feeder cells. In some embodiments, the PBMC are irradiated with gamma rays in the range of 3000 to 3600 rads to prevent cell division. The order of addition of the T cells and feeder cells to the culture media can be reversed, if desired. The culture can typically be incubated under conditions of temperature and the like that are suitable for the growth of T lymphocytes. For the growth of human T lymphocytes, for example, the temperature will generally be at least 25 degrees Celsius, at least 30 degrees C., or at least 37 degrees C.

The T lymphocytes expanded can include CD8+ cytotoxic T lymphocytes (CTL) and CD4+ helper T lymphocytes that are specific for an antigen present on a human tumor or a pathogen. Optionally, the expansion method can further comprise the step of adding non-dividing EBV-transformed lymphoblastoid cells (LCL) as feeder cells. LCL can be irradiated with gamma rays in the range of 6000 to 10,000 rads. The LCL feeder cells can be provided in any suitable amount, such as a ratio of LCL feeder cells to initial T lymphocytes of at least 10:1. Optionally, the expansion method can further comprise the step of adding anti-CD3 and/or anti CD28 antibody to the culture medium (e.g., at a concentration of at least 0.5 ng/ml). Optionally, the expansion method can further comprise the step of adding IL-2 and/or IL-15 to the culture medium (e.g., wherein the concentration of IL-2 is at least 10 units/ml). After isolation of T lymphocytes, both cytotoxic and helper T lymphocytes can be sorted into naive, memory, and effector T cell subpopulations either before or after expansion.

In some embodiments, CD8+ T cells obtained by standard methods are further sorted into naive, central memory, and effector memory cells by identifying cell surface antigens that are associated with each of those types of CD8+ T cells. In some embodiments, memory T cells are present in both CD62L+ and CD62L− subsets of CD8+ peripheral blood lymphocytes. PBMC are sorted into CD62L− CD8+ and/or CD62L+CD8+ fractions after staining with anti-CD8 and anti-CD62L antibodies. In some alternatives, the expression of phenotypic markers of central memory (TCM) include CD45RO, CD62L, CCR7, CD28, CD3, and/or CD127 and the cells are negative or low for granzyme B and/or CD45RA. In some alternatives, central memory T cells are CD45RO+, CD62L+, or CD8+ T cells. In some alternatives, effector (TE) are negative for CD62L, CCR7, CD28, and/or CD127, and positive for granzyme B and/or perforin. In some embodiments, naive CD8+ T lymphocytes are characterized by the expression of phenotypic markers of naive T cells including CD62L, CCR7, CD28, CD3, CD 127, and/or CD45RA.

Cytotoxic T lymphocytes (CTL) are T lymphocytes that express CD8 on their surface (e.g., a CD8+ expressing T cell, also referred to as a CD8+ T cell or a CD8 T cell, all of which can be used interchangeably). In some embodiments, such cells are also referred to as “memory” T cells, that are antigen-experienced. (Strictly from a nomenclature standpoint, a CD4-expressing T cell is also interchangeably referred to herein as a CD4+ T cell or a CD4 T cell; similar conventions apply when referring to other markers).

Central memory T cells (TCM) are antigen experienced CTLs that express CD62L or CCR-7 and/or CD45RO on their surface, and do not express or have decreased expression of CD45RA as compared to naive cells. In some alternatives, central memory cells are positive for expression of CD62L, CCR7, CD28, CD127, CD45RO, and/or CD95, and/or have decreased expression of CD54RA as compared to naive cells.

Effector memory T cells (or TEM) are antigen experienced T cells that do not express or have decreased expression of CD62L on their surface as compared to central memory cells, and do not express or have decreased expression of CD45RA as compared to a naive cell. In some alternatives, effector memory cells are negative for expression of CD62L and/or CCR7, as compared to naive cells or central memory cells, and have variable expression of CD28 and/or CD45RA.

Naive T cells are non-antigen-experienced T lymphocytes that express CD62L and/or CD45RA, and/or do not express CD45RO− as compared to central or effector memory cells. In some embodiments, naive CD8+ T lymphocytes are characterized by the expression of phenotypic markers of naive T cells including CD62L, CCR7, CD28, CD127, and/or CD45RA.

Effector (TE) T cells are antigen-experienced cytotoxic T lymphocyte cells that do not express or have decreased expression of CD62L, CCR7, CD28, and/or are positive for granzyme B and/or perforin, as compared to central memory or naive T cells.

Lymphoid precursor cells can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8, and are therefore classed as double-negative (CD4-CD8-) cells. As they progress through their development, they become double-positive thymocytes (CD4+CD8+), and finally mature to single-positive (CD4+CD8− or CD4−CD8+) thymocytes that are then released from the thymus to peripheral tissues. About 98% of thymocytes die during the development processes in the thymus by failing either positive selection or negative selection, whereas the other 2% survive and leave the thymus to become mature immunocompetent T cells.

CD4+ T helper cells are sorted into naive, central memory, and effector cells by identifying cell populations that have particular cell surface antigens. CD4+ lymphocytes can be obtained by standard methods. In some embodiments, naive CD4+ T lymphocytes are CD45RO−, CD45RA+ and CD62L+. In some embodiments, central memory CD4+ cells are CD62L+ and/or CD45RO+. In some embodiments, effector CD4+ cells are CD62L− and/or CD45RO−.

In some embodiments, populations of CD4+ and CD8+ that are antigen specific can be obtained by stimulating naive or antigen specific T lymphocytes with antigen. As but one example, antigen-specific T cell lines or clones can be generated to cytomegalovirus antigens by isolating T cells from infected subjects and stimulating the cells in vitro with the same antigen. Naive T cells can also be used. Any number of antigens from tumor cells can be utilized as targets to elicit T cell responses. In some embodiments, adoptive cellular immunotherapy compositions are useful in the treatment of a disease or disorder including a solid tumor (e.g., breast cancer, melanoma, among others), hematologic malignancy or other cancer.

In some embodiments, each of the CD4 or CD8 T lymphocytes can be sorted into naive, central memory, effector memory or effector cells prior to transduction as described herein. In other embodiments, each of the CD4 or CD8 T lymphocytes can be sorted into naive, central memory, effector memory, or effector cells after transduction.

Hematopoietic stem cells (HSCs) are precursor cells that can give rise to myeloid cells such as, for example, macrophages, monocytes, macrophages, neutrophils, basophils, eosinophils, erythrocytes, megakaryocytes/platelets, dendritic cells and lymphoid lineages (such as, for example, T-cells, B-cells, NK-cells). HSCs have a heterogeneous population in which three classes of stem cells exist, which are distinguished by their ratio of lymphoid to myeloid progeny in the blood (L/M).

Whether a cell or cell population is positive for a particular cell surface marker can be determined by flow cytometry using staining with a specific antibody for the surface marker and an isotype matched control antibody. A cell population “negative” for a marker refers to the absence of significant staining of the cell population with the specific antibody above the isotype control, whereas “positive” refers to uniform staining of the cell population above the isotype control. In some embodiments, a decrease in expression of one or markers refers to loss of 1 log 10 in the mean fluorescence intensity and/or decrease of percentage of cells that exhibit the marker of at least 20% of the cells, 25% of the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 20 and 100% when compared to a reference cell population. In some embodiments, a cell population positive for one or markers refers to a percentage of cells that exhibit the marker of at least 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 50 and 100% when compared to a reference cell population.

### Induced Pluripotent Stem Cells

Induced pluripotent stem (iPS) cell technology has the advantage of providing isogenic cells for cell therapy that do not provoke an immune response to treat and/or prevent a disease, such as cancer or autoimmune disease, among others. It is contemplated that iPS cells can provide a source of cells, including cells autologous to the subject to be treated, that can be genetically modified and differentiated (or differentiated and genetically modified) to a cell type useful for delivery of a drug copolymer as described herein. iPS cells are artificially derived from a non-pluripotent cell, typically an adult somatic cell. This was first demonstrated by Yamanaka et al., who transfected mouse fibroblasts with four genes (Oct4, Sox2, c-Myc, Klf4) to obtain iPS cells in vitro. Subsequently, iPS cells have been derived from human adult somatic cells. (Takahashi et al. Cell, 131:861-872 (2007); Yu et al. Science, 318:1917-1920, 2007).

Depending on the subject to be treated, the iPS cell can be a mammalian cell, for example a mouse, human, rat, bovine, ovine, horse, hamster, dog, guinea pig, or non-human primate cell. For example, if the ultimate goal is to generate therapeutic cells for transplantation into a patient, cells from that patient are desirably used to generate the iPS cells (e.g., autologous transplant). In one embodiment, the iPS cell is a human iPS cell.

Somatic cells useful for creating iPS cells can be obtained from any suitable source and can be any differentiated cell type. “Somatic cells,” as that term is used herein, refer to any cells forming the body of an organism, excluding germline cells. In certain embodiments, the somatic cells are obtained from blood, synovial fluid or from a tissue (e.g., skin). Exemplary somatic cells for reprogramming include, but are not limited to, blood cells, peripheral blood mononuclear cells (PBMC), or fibroblasts.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte, a cardiomyocyte, an immune cell, and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Essentially any method known in the art can be used for reprogramming somatic cells into iPS cells. When a composition is to be administered to a human, it is often preferred that the methods of reprogramming do not make lasting or permanent changes to the genome of the iPS cell, for example, by integrating a nucleic acid overexpressing a particular reprogramming factor. Exemplary methods include reprogramming using modified RNA, plasmids, non-integrating vectors, proteins or small molecules.

Reprogramming to an iPS phenotype can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors. These methods of re-programming may be preferred for cells to be used for therapeutic purposes, as they are less likely to provoke genomic damage likely to promote, e.g., cancer.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27− 275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from Biomol International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods and compositions described herein, isolated clones can be tested for the expression of a stem cell marker. Such expression in a cell derived from a somatic cell identifies the cell as an induced pluripotent stem cell. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry. Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; and other general markers for pluripotency, as known in the art.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Methods for cell culturing, developing, and differentiating pluripotent stem cells can be carried out with reference to standard literature in the field and are not described in detail herein. Those skilled in the art will appreciate that, except where explicitly required otherwise, iPS cells include primary tissue and established lines that bear phenotypic characteristics of iPS cells, and derivatives of such lines that still have the capacity of producing progeny of each of the three germ layers.

Differentiation of iPS Cells into Desired Cells

Methods are known in the art for differentiating iPS cells into a wide range of cell types, including immune cell types including antigen-specific T cells (see, e.g., Kaneko, Methods Mol. Biol. 1393: 67-73 (2016); Chang et al., PloS One 9(5): e97335 (2014); describes a broad repertoire of T cells derived from human iPS cells) and macrophages (see, e.g., Lachmann et al., Stem Cell Repts. 4(2): 282-296 (2015)). Methods for differentiating iPS cells to other cell types of interest for delivery of drug copolymer compositions are known to those of skill in the art.

One aspect of the technology described herein requires that the cells employed for delivery of a therapeutic agent encode a heterologous ligand-binding polypeptide that permits loading of a drug copolymer comprising the ligand onto the cell surface. Heterologous ligand-binding polypeptides for this purpose are described herein below.

### Heterologous Ligand-Binding Polypeptides

Heterologous ligand binding polypeptides that facilitate drug loading onto a cell as described herein can include any ligand-binding polypeptide domain that specifically binds a ligand that can be attached to or incorporated onto or into a drug copolymer composition. In order to avoid inadvertent localization of the cell bearing the receptor to a location other than that desired, it can be useful to use a ligand that is not normally expressed in the subject for this purpose. For example, while one might consider using streptavidin as a heterologous ligand binding polypeptide due to its strong binding to biotin under conditions commonly encountered in vivo, the natural occurrence of biotin in vivo may lead to the cells being drawn to unintended areas.

A class of heterologous ligand-binding polypeptides well-suited to the purpose of facilitating loading of ligand-containing drug copolymer compositions onto a cell includes the antigen-binding domains of antibodies that bind to artificial or synthetic antigen ligands. Examples of artificial or synthetic antigen ligands include (i) fluorescent proteins (e.g., fluorescein, rhodamine, etc.), (ii) affinity ligands (e.g., biotin or biotin acceptor domain (e.g., GLNDIFEAQKIEWHE), 9-cis retinoic acid, 8-aryl hydrocarbon, or sialic acid), (iii) peptide tags, such as polyhistidine (HHHHHH), c-Myc (EQKLISEEDL), human influenza agglutinin (HA) (YPYDVPDYA), FLAG (DYKDDDDK), thrombin fragment (LVPRGS), V5 (GKPIPNPLLGLDST), SB1 (PRPSNKRLQQ), Protein C fragment (EDQVDPRLIDGK), SV40 nuclear localization signal (PKKKRKVG), VSVG (YTDIEMNRLGK), Factor Xa (IDGR), or T7 (MASMTGGQQMG), (iv) small proteins, such as calmodulin binding protein, and (v) small molecule binders of intracellular proteins, such as CBP bromodomain ligands (see e.g., J Am Chem Soc (2014) 136(26), pg 9308-9319; DOI:10.1021/ja412434f). Antibodies that specifically bind these ligands are known in the art and/or can be purchased commercially from e.g., Sigma-Aldrich, abcam, Cell Signaling Technologies and New England Biolabs, among others.

Antibodies for use in the methods and compositions described herein can be raised through any conventional method, such as through injection of immunogen into mice and subsequent fusions of lymphocytes to create hybridomas. Such hybridomas may then be used either (a) to produce antibody directly, or (b) to clone cDNAs encoding antibody fragments for subsequent genetic manipulation. To illustrate one method employing the latter strategy, mRNA is isolated from the hybridoma cells, reverse-transcribed into cDNA using antisense oligo-dT or immunoglobulin gene-specific primers, and cloned into a plasmid vector. Clones are sequenced and characterized. They may then be engineered according to standard protocols to combine the heavy and light chains of each antibody, separated by a short peptide linker, into a bacterial or mammalian expression vector to produce a recombinant antibody which can then be expressed according to well-established protocols in mammalian cells (Kufer et al, 2004; Antibody Engineering: A Practical Approach, McCafferty, Hoogenboom and Chiswell Eds, IRL Press 1996). Antibodies, or other proteinaceous affinity molecules such as peptides, may also be created through display technologies that allow selection of interacting affinity reagents through the screening of very large libraries of, for example, immunoglobulin domains or peptides expressed by bacteriophage (Antibody Engineering: A Practical Approach, McCafferty, Hoogenboom and Chiswell Eds, IRL Press 1996). Antibodies useful in the methods and compositions described herein can also be humanized through grafting of human immunoglobulin domains, made from transgenic mice or bacteriophage libraries that have human immunoglobulin genes/cDNAs and/or can be purchased commercially from e.g., Sigma-Aldrich, abcam, Cell Signaling Technologies and New England Biolabs, among others.

Monoclonal antibodies that specifically bind a number of suitable antigens are known to, or can be raised by, one of ordinary skill in the art. The ordinarily-skilled artisan can readily identify and isolate the nucleic acid sequences encoding the antigen-binding variable domains of a given antibody from DNA of a hybridoma that expresses the antibody. For example, primers for the amplification and cloning of the VH and VL domains of a mammalian antibody are available and can be used on DNA from the hybridoma to amplify and clone the relevant domains. Techniques for the assembly of the cloned VH and VL domains with a spacer to generate a single chain antibody (most commonly, a single-chain Fv fragment or scFv) are well known in the art. One approach for expressing the heterologous ligand-binding polypeptide on the surface of a cell simply replaces the extracellular domain of a naturally-occurring cell surface protein (e.g., EGFR, VEGFR, PDGFR, scavenger receptors (e.g., CD206, CD163), PD-L1, Toll like receptors, Cluster of Differentiation proteins, immunoglobulin containing proteins, SLAM family proteins (e.g., 2B4CD150, CD319 etc.), non-internalizing cytokine and TNF family receptors, or any protein engineered to be retained in the cell membrane and modular scFv for ligand binding added) with an scFv specific for the chosen ligand, while another approach grafts the scFV-encoding sequence, with a peptide spacer as appropriate, onto a sequence encoding a membrane insertion sequence/transmembrane polypeptide domain, to anchor the resulting polypeptide to the cell surface.

In some embodiments of the methods and compositions described herein, a heterologous ligand-binding polypeptide can comprise proteinaceous structures other than antibodies that are able to bind to protein targets specifically, including but not limited to avimers (Silverman et al, 2005), ankyrin repeats (Zahnd et al., 2007) and adnectins (as described in U.S. Pat. No. 7,115,396), and other such proteins with domains that can be evolved to generate specific affinity for antigens, collectively referred to as “antibody-like molecules.” Modifications of proteinaceous heterologous ligand-binding polypeptides through the incorporation of unnatural amino acids during synthesis can be used to improve their properties (see Datta et al., 2002; and Liu et al., 2007). Such modifications can have several benefits, including the addition of chemical groups that facilitate subsequent conjugation reactions.

In certain embodiments, the heterologous polypeptide modulates an activity of a target cell, for example, influences the polarization of an immune cell. In one embodiment, a small molecule or drug that influences polarization of an immune cell includes, but is not limited to, an IDO inhibitor (e.g., INCB24360), a CSF1 inhibitor (e.g., BLZ945), a RON inhibitor (e.g., BMS-777607), a TLR9 agonist (e.g., VTX-2337), a CCR5 agonist (e.g., maraviroc), a CXCR1/CXCR2 inhibitor (e.g., reparixin), a CXCR4 blocker/antagonist (e.g., plerixafor), a CCR2 blocker/antagonist (e.g., PF-6309), an EP4 receptor blocker/antagonist (e.g., RQ-1586), a P2Y11 inhibitor (e.g., NF340), or an IL-13 inhibitor (e.g., tadalafil). In another embodiment, a drug or agent that influences macrophage or T cell polarization includes, but is not limited to, PI103, and resiquimod.

In some embodiments, the heterologous ligand-binding polypeptide is a peptide aptamer. A peptide aptamer is a peptide molecule that specifically binds to a target protein, and often interferes with the function of that target protein (Kolonin et al., Proc. Natl. Acad. Sci. USA 95:14266 (1998). Peptide aptamers consist of a variable peptide loop attached at both ends of a protein scaffold. Such peptide aptamers can often have a binding affinity comparable to that of an antibody (nanomolar range). Due to the highly selective nature of peptide aptamers, they can be used not only to target a specific protein, but also to target specific functions of a given protein (e.g., a signaling function).

Peptide aptamers are usually prepared by selecting the aptamer for its binding affinity with the specific target from a random pool or library of peptides. Peptide aptamers can be isolated from random peptide libraries by yeast two-hybrid screens (Xu et al., Proc. Natl. Acad. Sci. USA 94:12473 (1997). They can also be isolated from phage libraries (Hoogenboom et al., Immunotechnology 4:1 (1998) or chemically generated peptides/libraries.

### Heterologous Receptors

Other modifications to a cell useful to deliver a copolymer drug composition as described herein can include, for example, modification to express a heterologous receptor that binds a cell-surface ligand on a target cell. Expression of such a receptor can direct the cell to localize to and bind a chosen target cell. When the receptor binds a tumor antigen, as but one example, the cell expressing the heterologous receptor can be directed to the location of a cell or tumor expressing the tumor antigen. As with heterologous ligand-binding polypeptides discussed above, such heterologous receptors can include the antigen-binding domains of an antibody (e.g., as an scFv) that specifically binds the chosen cell-surface ligand or tumor antigen. Further, such heterologous receptors can be generated by simply replacing the extracellular domain of a naturally-occurring cell-surface receptor with sequence encoding a chosen scFv and a spacer as appropriate. Alternatively, such a receptor can be assembled from known sequences encoding an (optional) intracellular domain, a transmembrane domain, a spacer as appropriate and the chosen scFv sequence. Other ligand-binding moieties that specifically bind a cell-surface ligand on a target cell can include, for example, aptamers, avimers, ankyrin repeats, adnectins and other such antibody-like molecules as discussed above for heterologous ligand binding molecules.

In some embodiments, the heterologous receptor ligand is a peptide. In some embodiments, the peptide chain is a bispecific peptide. Peptides can readily be made and screened to create affinity reagents that recognize and bind to macromolecules such as proteins (see, for example, “Phage display of combinatorial peptide and protein libraries and their applications in biology and chemistry”. Current Topics in Microbiology and Immunology, vol. 243 1999, p. 87-105).

In some embodiments, a heterologous receptor ligand can be one or more oligosaccharides. Certain oligosaccharides are known ligands for certain extracellular or cell surface receptors. For example, Collins et al. describe a synthetic sialoside with affinity for cellular protein CD22. (“High-Affinity Ligand Probes of CD22 Overcome the Threshold Set by cis Ligands to Allow for Binding, Endocytosis, and Killing of B Cells” Collins et al., J. Immunol. 777:2994-3003, 2006).

### Chimeric Antigen Receptors

As noted above, in one embodiment, the cell is a T cell, modified not only to express a heterologous ligand binding polypeptide that permits binding of a drug copolymer composition to the cell, but also to express a chimeric antigen receptor that binds to a chosen cell-surface antigen expressed by a target cell. The chimeric antigen receptor re-directs the T cell's effector activity to a target cell expressing the chosen cell-surface antigen; engagement of the chimeric antigen receptor with the target cell surface antigen triggers the T cell's effector function against that target cell. In some embodiments, chimeric antigen receptors comprise: a ligand binding domain that specifically binds and/or targets a tumor cell surface molecule; a polypeptide spacer region; a transmembrane domain; and an intracellular signaling domain. In some embodiments, the ligand binding domain is a single-chain antibody fragment (scFv) that includes the variable heavy (VH) and variable light (VL) chains of a monoclonal antibody (mAb). Costimulatory signals can also be provided through the chimeric receptor by fusing the costimulatory domain of CD28 and/or 4-1BB to the CO3C chain. Chimeric receptors are specific and/or target cell surface molecules independent from HLA, thus overcoming the limitations of TCR-recognition including HLA-restriction and low levels of HLA-expression on tumor cells. Numerous variations on the general chimeric antigen receptor approach to re-targeting T cell effector functions are known in the art and can be applied within the scope of the methods and compositions described herein.

It has been recognized that in some instances, the length of the spacer between the ligand-binding domain and the transmembrane domain can affect the efficiency of binding by the ligand-binding domain to the target ligand and the efficiency of treatment via, e.g., CAT-T cells. A spacer as described herein can refer to a polypeptide chain that can range in length from a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. A spacer can comprise any 20 amino acids, for example, in any order to create a desirable length of polypeptide chain in a chimeric antigen receptor, which includes the amino acids arginine, histidine, lysine, leucine, aspartic acid, glutamic acid, serine, threonine, asparagine, glutamine, cysteine, glycine, proline, alanine, valine, isoleucine, methionine, phenylalanine, tyrosine and/or tryptophan. A spacer sequence can be a linker between an scFv and a transmembrane domain of a chimeric antigen receptor. In some alternatives of a method of making genetically modified T-cells which have a chimeric antigen receptor, the vector used in genetically making the T cells further comprises a sequence encoding a spacer as described herein.

It has been shown that the length of the spacer region affects the in vivo efficacy of T cells modified to express the chimeric receptor (CAR T cells) and can be customized for individual target molecules for optimal tumor or target cell recognition. As one example, a CD171 specific, targeting chimeric receptor with a spacer domain of 229 amino acids had less in vivo antitumor activity than a CD171-specific, targeting chimeric receptor with a short spacer region comprised of 15 amino acids or less (but not less than 1 or 2 amino acids).

In some embodiments, the chimeric receptor nucleic acid comprises a polynucleotide coding for a customizable spacer region selected from a library of polynucleotides coding for spacer regions. In some embodiments, a spacer length is selected based upon the location of the binding region and/or epitope, affinity of the antibody for the binding region and/or epitope, and/or the ability of the T cells expressing the chimeric receptor to proliferate in vitro and/or in vivo in response to antigen recognition.

Typically, a spacer region is found between the ligand binding domain and the transmembrane domain of the chimeric receptor. In some embodiments, a spacer region provides for flexibility of the ligand binding domain. In some embodiments, a spacer region has at least 10 to 229 amino acids, 10 to 200 amino acids, 10 to 175 amino acids, 10 to 150 amino acids, 10 to 125 amino acids, 10 to 115 amino acids, 10 to 100 amino acids, 10 to 75 amino acids, 10 to 50 amino acids, 10 to 40 amino acids, 10 to 30 amino acids, 10 to 20 amino acids, or 10 to 15 amino acids, or a length within a range defined by any two of the aforementioned amino acid lengths. In some embodiments, a spacer region has 15 amino acids or less (but not less than 1 or 2 amino acids), 119 amino acids or less (but not less than 1 or 2 amino acids), or 229 amino acids or less (but not less than 1 or 2 amino acids).

In some embodiments, the spacer region is derived from a hinge region of an immunoglobulin-like molecule. In some embodiments, a spacer region comprises all or a portion of the hinge region from a human IgG1, human IgG2, a human IgG3, or a human IgG4, or modified variant thereof, and can contain one or more amino acid substitutions or deletions. In some embodiments, a portion of the hinge region includes the upper hinge amino acids found between the variable heavy chain and the core, and the core hinge amino acids including a polyproline region. Typically, the upper hinge region has 3, 4, 5, 6, 7, 8, 9, or 10 amino acids.

In some embodiments, hinge region sequences can be modified at one or more amino acids in order to avoid undesirable structural interactions such as dimerization. In another embodiment, the spacer region comprises a portion of a modified human hinge region from IgG4.

In some embodiments, all or a portion of the hinge region is combined with one or more domains of a constant region of an immunoglobulin. For example, a portion of a hinge region can be combined with all or a portion of a CH2 or CH3 domain or variant thereof. In some embodiments, the spacer region does not include the 47-48 amino acid hinge region sequence from CD8 alpha, a full length Fc receptor, and/or the spacer region consisting of an extracellular portion of the CD28 molecule.

In some embodiments, a short spacer region has 15 amino acids or less (but not less than 1 or 2 amino acids) and comprises all or a portion of a IgG4 hinge region sequence or variant thereof, an intermediate spacer region has 119 amino acids or less (but not less than 1 or 2 amino acids) and comprises all or a portion of a IgG4 hinge region sequence and a CH3 region or variant thereof, and a long spacer has 229 amino acids or less (but not less than 1 or 2 amino acids) and comprises all or a portion of a IgG4 hinge region sequence, a CH2 region, and a CH3 region or variant thereof.

A polynucleotide coding for a spacer region can be readily prepared by synthetic or recombinant methods from the amino acid sequence. In some alternatives, a polynucleotide coding for a spacer region is operably linked to a polynucleotide coding for a transmembrane region. In some embodiments, the polynucleotide coding for the spacer region may also have one or more restriction enzyme sites at the 5′ and/or 3′ ends of the coding sequence in order to provide for easy excision and replacement of the polynucleotide with another polynucleotide coding for a different spacer region. In some embodiments, the polynucleotide coding for the spacer region is codon optimized for expression in mammalian cells, preferably humans.

In some embodiments, a library of polynucleotides, each coding for different spacer region is provided. In some embodiments, the spacer region is selected from the group consisting of a hinge region sequence from IgG1, IgG2, IgG3, or IgG4 or portion thereof, a hinge region sequence from IgG1, IgG2, IgG3, or IgG4 in combination with all or a portion of a CH2 region or variant thereof, a hinge region sequence from IgG1, IgG2, IgG3, or IgG4 in combination with all or a portion of a CH3 region or variant thereof, and a hinge region sequence from IgG1, IgG2, IgG3, or IgG4 in combination with all or a portion of a CH2 region or variant thereof, and a CH3 region or variant thereof. In some embodiments, a short spacer region is a modified IgG4 hinge sequence having 15 amino acids or less (but not less than 1 or 2 amino acids), an intermediate sequence is a IgG4 hinge sequence with a CH3 sequence having 119 amino acids or less (but not less than 1 or 2 amino acids); or a IgG4 hinge sequence with a CH2 and CH3 region having 229 amino acids or less (but not less than 1 or 2 amino acids).

### Further Cell Modifications

In various embodiments, the genetically engineered cell comprising the drug copolymer composition is further modified. For example, the cell can also include modifications that enhance or improve the efficacy of therapy by promoting the viability and/or function of transferred cells, or that provide a genetic marker to permit selection and/or evaluation of in vivo survival or migration, or that incorporate functions that enhance or improve the safety of cell-mediated therapy or immunotherapy, for example, by making the cell susceptible to negative selection in vivo as described by Lupton S. D. et al., Mol. and Cell Biol, 11:6 (1991); and Riddell et al., Human Gene Therapy 3:319-338 (1992). In some embodiments, expression of a desired gene product from a genetically engineered cell as described herein is inducibly controlled by the release of a therapeutic agent also delivered by the cell. Since the expression of the desired gene product stops when the therapeutically agent from the delivered cells is exhausted, treatment with such cells can be terminated at a desired time point and/or the expression of the gene product from the cell is self-limiting. The various modifications can be carried out in accordance with known techniques (see, e.g., U.S. Pat. No. 6,040,177 to Riddell et al.) or variations thereof that will be apparent to those skilled in the art based upon the present disclosure.

### Introducing Genetic Modifications to Cells

The methods and compositions described herein rely, in part, upon the ability to genetically modify cells to express, e.g., a heterologous ligand-binding polypeptide to permit binding of a drug copolymer composition to the cells, and in certain embodiments, to express a heterologous receptor that binds a cell-surface ligand on a target cell and/or to express a heterologous polypeptide that influences an activity of a target cell. Methods of introducing heterologous genetic material to cells are well known, and include, as non-limiting examples, calcium phosphate precipitation, liposome-mediated transfection, viral vector transduction and electroporation. The approach best suited to a give cell type will be known to those of ordinary skill in the art.

The genetic modification can be stably integrated into the genome or can be maintained episomally, e.g., on a plasmid or other episomal vector.

In some embodiments, a sequence directing the expression of a transgene can be placed under the control of naturally-occurring regulatory elements in the cell. In other embodiments, constructs for the expression of a heterologous polypeptide will generally include regulatory elements, including, promoters, enhancers, etc. that direct the expression of the encoded sequences. Such regulatory elements can include, where desired, cell-type specific regulatory elements. A gene under the control of a set of regulatory elements is generally referred to as “operably linked” to those elements. Typically, an expression vector comprises a transcription promoter, a gene encoding sequence, and a transcription terminator.

Genetic modification can be targeted, e.g., to a specific location of the genome, via the widely practiced CRISPR approach or one of the many variations thereof. Alternatively, genetic modifications can be randomly inserted into the genome; care should be taken to evaluate cells resulting from random integration of genetic modifications for their ability to cause tumors before administration to a subject for therapy.

Common techniques for genetic modification of mammalian cells use viral vectors including, for example, adenoviral vectors, adeno-associated viral (AAV) vectors, lentiviral vectors and retroviral vectors that infect the desired cell type, and viral vector transduction is the preferred approach for modifying, e.g., T cells, among others. Hematopoietic and lymphoid cells can be transduced, for example, via viral vectors, as well as via calcium phosphate precipitation, protoplast fusion and electroporation. Primary T cells have been successfully transduced by electroporation and by retroviral or lentiviral infection, which provide high transduction efficiencies. Retroviral or lentiviral integration takes place in a controlled fashion and results in the stable integration of one or a few copies of the new genetic information per cell.

An expression vector, or a vector, as described herein, is a nucleic acid molecule encoding a gene that is expressed in a host-cell. Typically, an expression vector comprises a transcription promoter, a gene encoding sequence, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

In some embodiments it may be useful to include in the transduced cells a positive marker that permits the selection of cells in vitro. The positive selectable marker may be a gene that upon being introduced into the host cell expresses a dominant phenotype permitting positive selection of cells carrying the gene. Genes of this type are known in the art, and include, inter alia, hygromycin-B phosphotransferase gene (hph), which confers resistance to hygromycin B, the amino glycoside phosphotransferase gene (neo or aph) from Tn5, which codes for resistance to the antibiotic G418, the dihydrofolate reductase (DHFR) gene, which codes for resistance to the antifolate inhibitor WR99210, and the adenosine deaminase gene (ADA), which permits selection by growth in the presence of low concentrations of the ADA inhibitor 2′-deoxycoformycin, with cytotoxic concentrations of adenosine, among others.

In some embodiments, the transduced cells comprise a transgene that expresses a desired gene product. Further, in some embodiments, the transgene is inducibly expressed in the presence of a therapeutic agent. The therapeutic agent can be administered separately from the cell composition or can be attached to the cells to be delivered as described herein. It is further contemplated herein that the release of a therapeutic agent from a cell composition as described herein can be controlled based on the desired delivery rate and/or length of treatment desired.

In some embodiments, the cell composition as described herein is transduced with a nucleic acid encoding a cytokine (e.g., macrophage cytokine such as IL-1, IL-6, IL-7, IL-12, IL-15, IL-17, IL-18, IL-21, IL-23, GM-CSF, TNFa, Type I and II interferons) among others, or with a nucleic acid encoding checkpoint blockades (PD-1, CTLA-4, B7-H4), CD28 agonist, 41BBL and/or 2B4, among others.

In other embodiments, the cell composition as described herein is transduced with a nucleic acid encoding an immune stimulatory viral or bacterial protein/peptide (e.g., TLR5 agonist (e.g., entolimod, HMGB1, HSP90, DAMPs, PAMPs, etc.).

In another embodiment, a cell composition as described herein can be used for the treatment of e.g., autoimmune disease and can be transduced to express one or more TNF inhibitors (e.g., ENBREL™ (etanercept), REMICADE™ (infliximab), HUMIRA™ (adalimumab), golimumab OR certolizumab pegol).

In another embodiment, a cell composition as described herein can be used for the treatment of e.g., chronic inflammation (e.g., Crohn's disease) and can be transduced to express an NFκB inhibitor, a STAT6 inhibitor, an NKG2D blocker, or a TNF receptor signaling blocker.

In another embodiment, a cell composition as described herein can be used for e.g., regenerative medicine/wound healing and can be transduced to express, for example, VEGF, EGF, FGF, or G-CSF.

In another embodiment, a cell composition as described herein can be used, for example, to administer a corrective gene or for gene therapy to treat and/or prevent disease. As but one example, a cell composition for use in the treatment of cystic fibrosis can be transduced to express e.g., cystic fibrosis transmembrane conductance regulator (CFTR).

Loading an Engineered Cell with Drugamer

Cells engineered to express a heterologous ligand-binding polypeptide on their surface can be loaded with drug copolymer compositions as described herein by any of several approaches. In one approach, the cells are contacted in vitro with a drug copolymer that comprises a cognate ligand for the heterologous ligand binding polypeptide, such that the drug copolymers become bound to and displayed upon the surface of the cell. A step of washing to remove unbound drug copolymer or, alternatively, selecting cells that display the drug copolymer can be performed if necessary or so desired. In another approach, the cells are injected or otherwise administered to a subject, and the drug copolymer composition is separately administered to the subject. Administration can be intravenous, but other routes as applicable to the circumstances can also be used. In this approach, the cells become associated with the drug copolymer as they encounter the copolymer in the subject's system. The choice of which approach to use will depend upon the indication being treated and on the drug being administered—if, for example, systemic toxicity with the drug is a serious issue, pre-loading the cells may be the preferred choice.

Inducible Gene Expression from Transgene

In some embodiments, the cell compositions described herein comprise a transgene and a system for inducible expression of a gene product from the transgene. The concept of inducible gene expression is well known in the art and/or could be envisioned by one of skill in the art. As such, the mechanisms of inducible gene expression are not described at length herein.

An inducible promoter/system useful in the methods and systems as disclosed herein can be induced by one or more physiological conditions, such as changes in pH, temperature, cell surface binding, and the concentration of one or more extrinsic or intrinsic inducing agents. The extrinsic inducer or inducing agent can comprise amino acids and amino acid analogs, nucleic acids, protein transcriptional activators and repressors, cytokines, hormones, and combinations thereof.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), doxacycline- or tetracycline-dependent regulatory systems, etc.

### Inducible Gene Product

Essentially any desired gene product can be inducibly expressed from a cell composition comprising a transgene, as described herein. The desired gene product can be e.g., an miRNA, an siRNA, an shRNA, a peptide, a protein, a ligand, an aptamer, an mRNA, among others.

A desired gene product will generally be one that comprises a desired function such as e.g., a reporter molecule and/or program, a product that mediates targeted cell killing, a gene therapy, an activating agent that acts reciprocally on the cell composition, or an agent that modulates a targeted cell microenvironment (e.g., a tumor microenvironment). An illustrative example of a gene product delivered to mediate targeted cell killing comprises expression of a pro-apoptotic factor (e.g., Bax and/or Bak that induce cytochrome c release from mitochondria), which can be delivered to a cell targeted for cell death (e.g., a tumor cell).

### Treatment Approaches

In one aspect, treatment of e.g., cancer, autoimmune disease or chronic infection can involve the administration of cells engineered to express on their cell surface at least one heterologous ligand-binding polypeptide, together with at least one copolymer drug composition, the at least one copolymer drug composition comprises a ligand that specifically binds the heterologous ligand-binding polypeptide, such that the at least one copolymer drug composition is displayed on the cell. The administered cell, together with the drug copolymer, can then track to a desired location. Such tracking can be an inherent function of the cell, as but one example being tracking of a neuronal stem cell to the brain, or tracking of a T cell to a cell that expresses a ligand recognized by a T cell receptor. Such tracking can alternatively be provided by another heterologous polypeptide expressed by the genetically engineered cell, such as a heterologous receptor that binds a given cell-surface protein expressed by a desired target cell.

The drug can be released from the drug copolymer in the microenvironment of the engineered cell. In this manner, the drug can achieve a high local concentration, while the drug accumulates to only minimal levels outside the vicinity of the engineered cell in vivo. The treatment approach described herein provides a versatile platform for treatment of cancer, autoimmune disease and infection, among other indications, by permitting not only the delivery of a drug to a desired location, but, when the cells themselves have effector functions either inherently or due to further genetic engineering, by delivering further functionality, such as immunosuppression, immunostimulation, enzyme activity to convert an inactive pro-drug to an active form at the desired site, or the direction of cytokine attack on the target cell, among others. Combinations of drug and biologic factors delivered to the location targeted by an engineered cell as described herein can provide additive and/or synergistic therapeutic effects.

In one embodiment, cells can be re-loaded with drug copolymer in vivo, e.g., by administering a dose of drug copolymer composition to an individual who has been treated with cells originally bearing drug copolymer, but in which the drug has substantially all been released or otherwise depleted. The heterologous ligand binding polypeptide expressed on the cells can bind and sequester or accumulate the drug copolymer at the cells' location to re-load the cells. In this manner, one can maintain or restore localized drug delivery even after the initial load of drug copolymer composition has declined or ceased.

In one embodiment, cells can be genetically engineered to express a desired gene product that is inducibly controlled by a drug. For example, in the presence of the drug expression of the desired gene product can be expressed, while in the absence of the drug such expression is inhibited. An alternative design can also be used in which the drug represses the expression of a transgene, such that expression of the transgene only occurs once the drug is exhausted. One application of such a design would use, for example a toxin-encoding transgene, such as an endonuclease—encoding transgene that would kill the cell when the drug is exhausted, thereby permitting removal of the cell when it is no longer needed. One of skill in the art will appreciate that there are multiple inducible expression systems known in the art and that can be applied to the methods and compositions as described herein.

In some embodiments, the drug that inducibly controls expression of the desired gene product is administered separately from the cell composition, thereby permitting the expression of the desired gene product to be turned on and/or off as necessary or as part of a treatment regime. This method permits rapid control of gene expression from the administered cells, including pulsed expression of the gene product.

The drugamer platform permits a broad range of adaptations to suit a particular application. In one aspect, for example, it is contemplated that a drugamer can, either on its own and by nature of the specific polymerization approach, or by combination with one or more additional agents, e.g., cross-linkers or viscosity increasing agents, form a gel. Such a gelled or gel-forming drugamer can include, for example, a small molecule or other drug agent in the drugamer polymer that regulates, for example, an activity of an engineered cell. Administration of the gelled or gel-forming drugamer at a site at which engineered cells are administered can establish a very location-specific niche in which activity of an engineered cell, e.g., expression and delivery of a biologic or recruitment of immune effector cells, among others, is specifically promoted. In one embodiment of this aspect, a drug comprised by the drugamer regulates expression of a transgene in the engineered cell. In one embodiment, the expression of the transgene is promoted only when the drug remains present, such that the depletion of the drugamer in the vicinity of the engineered cell limits the expression of the transgene to a desired or tunable timeframe. In these embodiments, it is contemplated that the engineered cell need not express a heterologous ligand-binding polypeptide that binds a ligand on the drugamer—the gelled or gel-forming drugamer can provide the drugamer's drug to the administered cells in a localized manner that does not require loading of the drugamer onto the cell via a drugamer ligand. However, it is also specifically contemplated that an engineered cell according to this aspect can further express a heterologous ligand binding polypeptide and be loaded with another drugamer or drugamers via interaction with a ligand on the drugamer(s).

### Cancers

Some non-limiting examples of cancer that can be treated using the methods and compositions described herein include, but are not limited to, carcinoma, lymphoma, blastoma, sarcoma, and leukemia. Other exemplary cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments, the carcinoma or sarcoma includes, but is not limited to, carcinomas and sarcomas found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. The types of carcinomas include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

In one embodiment of the methods and compositions described herein, the subject having the tumor, cancer or malignant condition is undergoing, or has undergone, treatment with a conventional cancer therapy. In some embodiments, the cancer therapy is chemotherapy, radiation therapy, immunotherapy or a combination thereof.

Exemplary anti-cancer agents that can be used in the methods and compositions described herein include alkylating agents such as thiotepa and CYTOXAN™; cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas, such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2− ethylhydrazide; procarbazine; PSK; polysaccharide complex (JHS Natural Products™, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vinde sine; dacarbazine; mannomustine; mitobronitol; mitolactol; pipobroman; gacyto sine; arabinoside (“Ara-C”); cyclophosphamide; thiotepa; taxoids, e.g., TAXOL™ paclitaxel (Bristol-Meyers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™, gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™, vinorelbine; novantrone; teniposide; edatrexate; daunomycin; aminopterin; xeloda; ibandronate; irinotecan (CAMPTOSAR™, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX™); lapatinib (TYKERB™); inhibitors of PKC-alpha, Raf, H-Ras, EGFR (e.g., erlotinib (TARCEVA™)) and VEGF-A that reduce cell proliferation, and pharmaceutically acceptable salts, acids or derivatives of any of the above. In addition, the methods of treatment can further include the use of radiation.

Such therapies can either directly target a tumor (e.g., by inhibition of a tumor cell protein or killing of highly mitotic cells) or act indirectly, e.g., to provoke or accentuate an anti-tumor immune response by modulating the tumor microenvironment.

Immune Checkpoint Inhibitors: The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals. In some embodiments, a subject or patient is treated with at least one inhibitor of an immune checkpoint protein.

Cytotoxic T-lymphocyte associated antigen 4 (CTLA-4) is an immune checkpoint protein that downregulates pathways of T-cell activation (Fong et al., Cancer Res. 69(2):609-615, 2009; Weber Cancer Immunol. Immunother, 58:823-830, 2009). Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells, thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In one embodiment, the anti-CTLA-4 antibody is ipilimumab (also known as 10D1, MDX-D010) a fully human monoclonal IgG antibody that binds to CTLA-4. Ipilimumab is marketed under the name YERVOY™ and has been approved for the treatment of unresectable or metastatic melanoma.

Further examples of checkpoint molecules that can be attached to a modified T cell lymphocyte include, but are not limited to, PDL2, B7-H3, B7-H4, BTLA, HVEM, GALS, VISTA, KIR, 2B4 (belongs to the CD2 family of molecules and is expressed on all NK, γδ, and memory CD8+ (αβ) T cells), CD160 (also referred to as BY55), A2aR, TIGIT, DD1-□, TIM-3, Lag-3, and various B-7 family ligands. B7 family ligands include, but are not limited to, B7-1, B7-2, B7-DC, B7-H1, B7-H2, B7-H3, B7-H4, B7-H5, B7-H6 and B7-H7.

Another immune checkpoint protein is programmed cell death 1 (PD-1). PD-1 limits the activity of T cells in peripheral tissues at the time of an inflammatory response to infection and limits autoimmunity. PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD1 blockade can be accomplished by a variety of mechanisms including antibodies that bind PD1 or its ligand, PD-L1. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In certain embodiments the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade. Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94).

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

Also specifically contemplated herein are agents that disrupt or block the interaction between PD-1 and PD-L1, such as a high affinity PD-L1 antagonist.

MEK and/or ERK inhibitors: In some embodiments of the methods described herein, an ERK inhibitor is delivered using an engineered cell as described herein to a subject having cancer. ERK is the only known substrate for MEK1 and MEK2. Phosphorylation of ERK results in translocation to the nucleus where it phosphorylates nuclear targets and regulates various cellular processes such as proliferation, differentiation, and cell cycle progression (J. L. Yap et al., Chem. Med. Chem. 2011 6:38).

The term “ERK inhibitors” as used herein relates to compounds capable of fully or partially preventing, or reducing or inhibiting ERK1/2 signaling activity. Inhibition can be effective at the transcriptional level, for example by preventing or reducing or inhibiting mRNA synthesis of ERK1 or ERK2 mRNA, for example, human ERK1 (NCBI reference NP-002737) or human ERK2 (NCBI reference NP-620407). Exemplary small molecule ERK inhibitors include, but are not limited to SCH772984, 3-(2-aminoethyl)-5-))4-ethoxyphenyl)methylene)-2,4-thiazolidinedione (PKI-ERK-005), CAY10561 (CAS 933786-58-4; CAYMAN CHEMICAL), and VTXX11e.

As used herein, the term “MEK inhibitors” refers to compounds capable of fully or partially preventing or reducing or inhibiting MEK signaling activity. Inhibition can be effective at the transcriptional level, for example, by preventing or reducing or inhibiting mRNA synthesis of mRNA encoding human MEK1 (NCBI reference NP-002746), or human MEK2 (NCBI reference NP109587). Exemplary small molecule inhibitors of MEK include, but are not limited to PD 98059, a highly selective inhibitor of MEK1 and MEK2 with IC50 values of 4 μM and 50 μM respectively (Runden E et al., J Neurosci 1998, 18(18) 7296-305), trametinib (GSK 120212), cobimetinib (XL518), MEK 162, RO5126766, GDC-0623, PD0325901 (Pfizer), Selumetinib, a selective MEK inhibitor (Astrazeneca/Array Biopharma, also known as AZD6244), ARRY-438162 (Array Biopharma), PD198306 (Pfizer), AZD8330 (Astrazeneca/Array Biopharma, also called ARRY-424704), PD184352 (Pfizer, also called CI-1040), PD 184161 (Pfizer), α-[Amino[(4-aminophenyl)thio]methylene]-2-(trifluoromethyl)benzeneacetonitrile (SL327), 1,4-Diamino-2,3-dicyano-1,4-bis(2− aminophenylthio)butadiene (U0126), Ro 09-2210 (Roche), RDEA1 19 (Ardea Biosciences), and ARRY-704 (Astrazeneca).Also contemplated are combination treatments for use with the modified T cells and methods described herein, the treatments comprising an ERK inhibitor and a MEK inhibitor.

Also specifically contemplated herein are inhibitors that inhibit or reduce the function of signaling pathway members upstream of ERK. Any of these upstream elements, if targeted, can also cause resistance that can be compensated by providing an ERK inhibitor. Exemplary pathway members include, but are not limited to, Ras, NF1, RASGAP1, RASGAP2, SPRY, GRB2, SOS, PAK1, KSR1, and KSR2.

Exemplary Ras kinase inhibitors include, for example, BMS-214662 (Bristol-Meyers Squibb), SCH 66336 (also known as Ionafarnib; Schering-Plough), L-778,123 (Merck), R115777 (also known as ZARNESTRA™ or Tipifarnib; Johnson & Johnson), and 6-[(4-chloro-phenyl)-hydroxy-(3-methyl-3H-imidazol-4-yl)-methyl]-4-(3-ethynyl-phenyl)-1-methyl-1H-quinolin-2-one (Osi Pharmaceuticals, Inc.). Additional Ras inhibitors are known to those of skill in the art and are not described in detail herein. Similarly, inhibitors of NF1, RASGAP1, RASGAP2, SPRY, GRB2, SOS, PAK1, KSR1, and KSR2 are known to those of skill in the art and are not described herein.

In some embodiments, one of skill in the art may choose to modulate the tumor microenvironment, for example, to improve tumor associated antigen presenting cell functions, reverse the phenotype and function of polarized innate immune cells that suppress T cells functions, and/or inhibit termination or prevention of cytotoxic immune cell activation.

Thus, in one embodiment, a combination of a GM-CSF (biologic), resiquimod (TLR7/8 agonist drugamer), and galunisertib can be used to modulate the tumor microenvironment. GM-CSF can restore impaired expression of the antigen-presenting proteins MHC class II and CD80/86. Antigen presentation is further augmented in response to cytokines produced by macrophages in response to resiquimod.

In another embodiment, a combination of PD-L1 scFvFc (biologic) and resiquimod (drugamer) can be used to modulate the tumor microenvironment. Binding of the PD-L1 scFvFc biologic to the surface of tumor and TAM PD-L1 will directly reduce suppression of T cell activation, and synergize with pro-inflammatory macrophage derived proteins produced in response to resiquimod that will improve ADCC. This combination is expected to show improved killing of both antigen expressing tumor cells by T cells, as well as those binding the scFvFc by NK cells, which express CD16.

### Autoimmune Disease

The term “autoimmune disease” as used herein is defined as a disorder that results from an inappropriate and excessive response to a self-antigen. Examples of autoimmune diseases include, but are not limited to, Addison's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

In some embodiments of the methods, compositions and treatments described herein, the autoimmune disease(s) to be treated or prevented include, but are not limited to, rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus), gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, and psoriasis. Autoimmune disease has also been recognized to encompass atherosclerosis and Alzheimer's disease.

In some embodiments of the methods of treating chronic immune conditions as described herein, the subject being administered the compositions described herein has or has been diagnosed with host versus graft disease (HVGD). In a further embodiment, the subject being treated with the methods described herein is an organ or tissue transplant recipient. In other embodiments of the methods of treating chronic immune conditions as described herein, the methods are used for increasing transplantation tolerance in a subject. In some such embodiments, the subject is a recipient of an allogenic transplant. The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

### Infectious Disease

Typically, “infectious disease” or inflammatory conditions related to infectious disease are from a microbial infection, for example, a bacterial infection, a eukaryotic parasitic infection, a viral infection, or a fungal infection or are related to systemic inflammatory response syndrome (SIRS). The infectious disease or inflammatory conditions that are related to infectious disease may be from bacteremia, viremia, or fungemia, or from septicemia due to any class of microbe.

In some embodiments, a clinical indicator can be used to assess infectious disease or inflammatory conditions related to infectious disease, for example, a clinical indicator can be selected from the group consisting of blood chemistry, urinalysis, X-ray or other radiological or metabolic imaging technique, other chemical assays, and physical findings.

In some embodiments, the subject may have presumptive signs of a systemic infection including at least one of: elevated white blood cell count, elevated temperature, elevated heart rate, and elevated or reduced blood pressure, relative to medical standards. In other embodiments, the inflammatory conditions related to infectious disease can be inflammatory conditions arising from at least one of blunt or penetrating trauma, surgery, endocarditis, urinary tract infection, bacterial infection, viral infection, fungal infection, pneumonia, or dental or gynecological examinations or treatments.

### Therapeutic Compositions

Following in vitro cell culture, isolation, or differentiation as described herein, engineered cells are prepared for treatment and/or implantation. The cells are suspended in a physiologically compatible carrier, such as cell culture medium (e.g., Eagle's minimal essential media), phosphate buffered saline, or a T cell lymphocyte specific medium. The volume of cell suspension to be implanted will vary depending on the site of implantation, treatment goal, and cell density in the solution.

Pharmaceutically acceptable carriers and diluents include saline, aqueous buffer solutions, solvents and/or dispersion media. The use of such carriers and diluents is well known in the art. The solution is preferably sterile and fluid. Solutions for use with the compositions and methods described herein can be prepared by incorporating the cells as described herein in a pharmaceutically acceptable carrier or diluent and, as required, other ingredients.

In other embodiments, the engineered cell composition comprises at least 1,000, at least 10,000, at least 100,000, at least 10⁶, at least 10⁷, at least 10⁸, at least 10⁹, at least 10¹⁰ engineered cells or more comprising a therapeutic agent on their surface. In one embodiment, the engineered cell composition comprises at least 100,000 engineered cells comprising a therapeutic agent on their surface. It will be appreciated by one of skill in the art that the number of cells can be tailored or optimized for efficacious treatment of the subject in need thereof. As such, one of skill in the art can perform dose escalation studies, if desired.

It will be appreciated by one of skill in the art that a cell composition useful for treating autoimmune disease, infectious disease or cancer does not need to be a pure, homogeneous culture of e.g., T lymphocytes. Accordingly, in one embodiment, the composition administered comprises at least 2% engineered cells (e.g., engineered T lymphocytes). In other embodiments, the composition comprises at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more engineered cells as described herein.

The cells can be administered to a subject by any appropriate route that results in delivery of the cells to a desired location in the subject where at least a portion of the cells remain viable. It is preferred that at least 5% remain viable. In other embodiments, at least 10%, at least 20%, at least 30%, at least 40%, or at least 50%, at least 60%, at least 70%, at least 80%, at least 90%, at least 95%, at least 98%, at least 99% or more of the cells remain viable after administration into a subject. The period of viability of the cells after administration to a subject can be as short as a few hours, e.g., twenty-four hours, to a few days, to as long as a few weeks to months.

To accomplish these methods of administration, the engineered cell composition(s) can be inserted into a delivery device that facilitates introduction by injection or implantation of the cells into the subject. Typically, the cells are injected into the target area as a cell suspension. Alternatively, the engineered cells can be embedded in a solid or semisolid support matrix when contained in such a delivery device.

Support matrices in which the engineered cells as described herein can be incorporated or embedded include matrices which are recipient-compatible and which degrade into products that are not harmful to the recipient. Natural and/or synthetic biodegradable matrices are examples of such matrices. Natural biodegradable matrices include, for example, collagen matrices. Synthetic biodegradable matrices include synthetic polymers such as polyanhydrides, polyorthoesters, and polylactic acid. These matrices provide support and protection for the cells in vivo.

In some embodiments, administration of a composition comprising engineered cells is repeated after a given interval of time (e.g., one day, three days, one week, two weeks, three weeks, one month or more. Repeated treatments can be performed, for example, to establish or maintain a threshold level of engraftment necessary to continue effective treatment, as necessary, of autoimmune disease, infectious disease or cancer. In some embodiments, the method is repeated twice, three times, four times, five times or more.

### Efficacy Measurement

The term “effective amount” as used herein refers to the amount of a population of engineered cells needed to alleviate at least one or more symptoms of autoimmune disease, infectious disease or cancer, and relates to a sufficient amount of a composition to provide the desired effect. An effective amount as used herein also includes an amount sufficient to prevent or delay the development of a symptom of the disease, alter the course of a symptom of the disease (for example but not limited to, slow the progression of a symptom of the disease, such as tumor growth), or reverse a symptom of the disease. It is understood that for any given case, an appropriate “effective amount” can be determined by one of ordinary skill in the art using routine experimentation. Given the intricacies of the brain and the unpredictable nature of cell engraftment, the “effective amount” of cells may vary among different patients, however one can easily determine in hindsight if the amount of cells administered was indeed an ‘effective amount.” Thus, further treatments can be modified accordingly.

The efficacy of treatment can be determined by the skilled clinician. However, a treatment is considered “effective treatment,” as the term is used herein, if any one or all of the symptoms, or other clinically accepted symptoms or markers of an autoimmune disease, infectious disease or cancer are reduced, e.g., by at least 10% following treatment with a composition comprising engineered cells as described herein. Methods of measuring these indicators are known to those of skill in the art and/or described herein.

In one embodiment, effective treatment is determined by a reduction in the dose of a conventional pharmacological treatment, required to maintain adequate control of symptoms of autoimmune disease, infectious disease or cancer.

In some embodiments, the subject is further evaluated using one or more additional diagnostic procedures, for example, by medical imaging, physical exam, laboratory test(s), clinical history, family history, gene test, BRCA test, and the like. Medical imaging is well known in the art. As such, the medical imaging can be selected from any known method of imaging, including, but not limited to, ultrasound, computed tomography scan, positron emission tomography, photon emission computerized tomography, and magnetic resonance imaging.

The present invention may be as described in any one of the following numbered paragraphs.

1. A composition comprising:

a) a genetically engineered cell that expresses on its cell surface at least one heterologous ligand-binding polypeptide; and

b) at least one copolymer drug composition, wherein each of the at least one copolymer drug composition comprises a ligand that specifically binds the heterologous ligand-binding polypeptide described in (a), such that the at least one copolymer drug composition is displayed on the surface of the genetically engineered cell,

wherein the engineered cell further comprises a nucleic acid construct encoding a polypeptide, operably linked to a regulatory nucleic acid sequence that renders expression of the polypeptide modulated by the presence or absence of a drug comprised by the copolymer drug composition.

2. The composition of paragraph 1, wherein the at least one heterologous ligand-binding polypeptide comprises an antigen binding domain of an antibody that binds the ligand comprised by a copolymer drug composition.

3. The composition of paragraph 1 or 2, wherein the genetically-engineered cell further expresses a heterologous receptor that binds a cell-surface ligand on a target cell.

4. The composition of paragraph 1, 2, or 3, wherein the genetically engineered cell is a T cell, a macrophage or a stem cell.

5. The composition of paragraph 4, wherein the stem cell is a hematopoietic stem cell or a neuronal stem cell.

6. The composition of paragraph 3, wherein the heterologous receptor that binds a cell-surface ligand on a target cell binds a tumor antigen expressed on a target cell.

7. The composition of paragraph 3, wherein the heterologous receptor that binds a cell-surface ligand on a target cell comprises a chimeric T cell receptor.

8. The composition of paragraph 3, wherein the heterologous receptor that binds a cell-surface ligand on a target cell comprises the antigen-binding domain of an antibody.

9. The composition of any one of paragraphs 1-8, wherein the drug comprised by the at least one copolymer drug composition comprises a small molecule drug.

10. The composition of paragraph 9, wherein the drug comprised by the copolymer drug composition is selected from the group consisting of doxycycline and tetracycline.

11. The composition of any one of paragraphs 1-10, wherein the drug comprised by the copolymer drug composition is required for the expression of the polypeptide

12. The composition of paragraph 11, wherein the copolymer drug composition comprises 4− hydroxytamoxifen, or CMP-8.

13. The composition of any one of paragraphs 1-12, wherein the nucleic acid construct comprises a sequence encoding a riboswitch that responds to a drug comprised by the copolymer drug composition.

14. The composition of any one of paragraphs 1-13, wherein the nucleic acid construct is regulated by chemically-induced dimerization, and wherein the copolymer drug composition comprises a chemical inducer of the chemically-induced dimerization.

15. The composition of paragraph 14, wherein:

a) the chemically-induced dimerization comprises FKBP homodimerization and the copolymer drug composition comprises FK1012;

b) the chemically-induced dimerization comprises FKBP dimerization with Calcineurin A and the copolymer drug composition comprises FK506;

c) the chemically-induced dimerization comprises FKBP dimerization with CyP-Fas and the copolymer drug composition comprises FKCsA;

d) the chemically-induced dimerization comprises FKBP dimerization with the FRB domain of mTOR and the copolymer drug composition comprises rapamycin;

e) the chemically-induced dimerization comprises GyrB homodimerization and the copolymer drug composition comprises couermycin; or

f) the chemically-induced dimerization comprises GAI heterodimerization with GID1 and the copolymer drug composition comprises gibberellin.

16. The composition of any one of paragraphs 1-15, wherein the expression of the polypeptide is repressed by the drug comprised by the copolymer drug composition.

17. The composition of paragraph 16, wherein the polypeptide promotes the death of the genetically engineered cell.

18. The composition of any one of paragraphs 1-18, wherein the polypeptide modulates an activity of a target cell.

19. The composition of paragraph 18, wherein the polypeptide comprises an immunomodulator, an inhibitor of a growth factor or a growth factor receptor

20. The composition of paragraph 19, wherein the immunomodulator comprises an immune checkpoint inhibitor, a cytokine, a chemokine, or a polypeptide that influences macrophage or T cell polarization.

21. The composition of paragraph 20, wherein the immunomodulator comprises an inhibitor of an immune checkpoint polypeptide selected from the group consisting of PD-1, PD-L1, TIM-3, CTLA4, TIGIT, KIR, LAG3, and/or DD1-α.

22. The composition of paragraph 20, wherein the immunomodulator comprises a cytokine or chemokine selected from the group consisting of: IL-1, IL-6, IL-7, IL-12, IL-15, IL-17, IL-18, IL-21, IL-23, GM-CSF, TNFa, Type I and II interferons, checkpoint blockades (PD-1, CTLA-4, B7-H4), CD28 agonist, 41BBL, and 2B4.

23. The composition of any one of paragraphs 1-22, wherein the polypeptide comprises an antigen-binding domain of an antibody.

24. The composition of any one of paragraphs 1-23, which comprises at least two different copolymer drug compositions as recited in (b).

25. The composition of paragraph 24, wherein the at least two different copolymer drugs both bind to and/or act on the regulatory nucleic acid sequence.

26. The composition of paragraph 24, wherein only one of the at least two different copolymer drugs binds to the regulatory nucleic acid sequence.

27. The composition of paragraph 26, wherein

(i) another of the at least two different copolymer drugs is a therapeutic agent, or

(ii) one of the at least two different copolymer drugs is a therapeutic agent.

28. The composition of paragraph 27, wherein the therapeutic agent comprises a kinase inhibitor, a growth factor receptor inhibitor, a chemotherapeutic, an estrogen receptor (ER) ligand, a Toll-Like Receptor (TLR) antagonist, an indoleamide 2,3dioxygenase inhibitor, a TGFβ receptor I (TβRI) inhibitor, and a cyclic dinucleotides (CDNs) STING agonist.

29. The composition of any one of paragraphs 1-28, further comprising a gel or matrix comprising one or more agents that acts upon the genetically engineered cell or a target cell thereof.

30. A method of administering a polypeptide of interest to an individual in need thereof, the method comprising administering to the individual a composition of paragraph 1, wherein a drug comprised by the copolymer drug composition is released from the copolymer after the composition is administered, and wherein that drug induces expression of the polypeptide wherein the expression of the polypeptide continues only while that drug is present.

31. The method of paragraph 30, wherein the at least one heterologous ligand-binding polypeptide comprises an antigen binding domain of an antibody that binds the ligand comprised by a copolymer drug composition.

32. The method of paragraph 30 or 31, wherein the genetically engineered cell is contacted with the at least one copolymer drug composition before the genetically engineered cell is administered.

33. The method of any one of paragraphs 30-32, wherein the genetically-engineered cell further expresses a heterologous receptor that binds a cell-surface ligand on a target cell.

34. The method of paragraph 33, wherein the target cell is a tumor cell and wherein the polypeptide of interest promotes tumor cell death.

35. The method of paragraph 34, wherein the polypeptide of interest comprises a toxin, an immunomodulator, a TRAIL polypeptide, an inhibitor of a growth factor or a growth factor receptor.

36. The method of any one of paragraphs 30-35, wherein the cell comprises at least two different copolymer drug compositions as recited in (b).

37. The method of paragraph 36, wherein at least one of the at least two different copolymer drug compositions comprises a kinase inhibitor, a growth factor receptor inhibitor, a chemotherapeutic, an estrogen receptor (ER) ligand, a Toll-Like Receptor (TLR) antagonist, an indoleamide 2,3dioxygenase inhibitor, a TGFβ receptor I (TβRI) inhibitor, and a cyclic dinucleotides (CDNs) STING agonist.

38. The method of any one of paragraphs 30-37, wherein the genetically engineered cell is a T cell, a macrophage or a stem cell.

39. The method of paragraph 38, wherein the stem cell is a hematopoietic stem cell or a neuronal stem cell.

40. The method of paragraph 38, wherein the heterologous receptor that binds a cell-surface ligand on a target cell binds a tumor antigen expressed on a target tumor cell.

41. The method of paragraph 33, wherein the heterologous receptor that binds a cell-surface ligand on a target cell comprises a chimeric T cell receptor.

42. The method of paragraph 33, wherein the heterologous receptor that binds a cell-surface ligand on a target cell comprises the antigen-binding domain of an antibody.

43. The method of any one of paragraphs 30-42, wherein the drug comprised by the at least one copolymer drug composition comprises a small molecule drug.

44. The method of paragraph 43, wherein the small molecule drug is selected from the group consisting of doxycycline and tetracycline.

45. The method of any one of paragraphs 30-44, wherein a drug comprised by the copolymer drug composition is required for the expression of the polypeptide of interest.

46. The method of any one of paragraphs 30-45, wherein the polypeptide of interest modulates an activity of a target cell.

47. The method of any one of paragraphs 30-46, wherein the polypeptide of interest comprises an immunomodulator, an inhibitor of a growth factor or a growth factor receptor.

48. The method of paragraph 47, wherein the immunomodulator comprises an immune checkpoint inhibitor, a cytokine, a chemokine, or a polypeptide that influences macrophage or T cell polarization.

49. The method of paragraph 47 wherein the immunomodulator comprises an inhibitor of an immune checkpoint polypeptide selected from the group consisting of PD-1, PD-L1, TIM-3, CTLA4, TIGIT, KIR, LAG3, and DD1-α.

50. The method of paragraph 47, wherein the immunomodulator comprises a cytokine or chemokine selected from the group consisting of IL-1, IL-6, IL-7, IL-12, IL-15, IL-17, IL-18, IL-21, IL-23, GM-CSF, TNFa, Type I and II interferons, checkpoint blockades (PD-1, CTLA-4, B7-H4), CD28 agonist, 41BBL, and 2B4.

51. The method of any one of paragraphs 30-50, wherein the polypeptide of interest comprises an antigen-binding domain of an antibody.

52. The method of any one of paragraphs 30-51, wherein the genetically engineered cell is administered in a gel or matrix comprising one or more agents that acts upon the genetically engineered cell or a target cell thereof.

53. A method of limiting the duration of a cell-mediated therapy in an individual in need thereof, the method comprising administering to the individual a composition comprising:

a) a genetically engineered cell that expresses on its cell surface at least one heterologous ligand-binding polypeptide; and

b) at least one copolymer drug composition, wherein each of the at least one copolymer drug composition comprises a ligand that specifically binds a heterologous ligand-binding polypeptide described in (a), such that the at least one copolymer drug composition is displayed on the surface of the genetically engineered cell,

wherein the engineered cell further comprises a nucleic acid construct encoding a first polypeptide of interest, operably linked to a regulatory nucleic acid sequence that renders expression of the first polypeptide of interest sensitive to the presence or absence of a drug comprised by a copolymer drug composition described in (b),

wherein the engineered cell further comprises a nucleic acid construct encoding a second polypeptide of interest,

wherein a drug comprised by a copolymer drug composition as described in (b) is released from the copolymer after the composition is administered, wherein a drug released from a copolymer composition represses the expression of the first polypeptide of interest,

wherein the second polypeptide of interest is a therapeutic polypeptide, and

wherein the first polypeptide of interest promotes the death of the genetically engineered cell.

54. The method of paragraph 53, wherein the first polypeptide comprises a toxin polypeptide.

55. The method of paragraph 54, wherein the toxin polypeptide comprises a restriction endonuclease, a cytolytic peptide, Apoptosis-Inducing Factor (AIF), a caspase polypeptide, or a diphtheria toxin A fragment.

56. The method of any one of paragraphs 53-55, wherein the drug that represses expression of the first polypeptide is selected from the group consisting of tetracycline and doxycycline.

57. A composition comprising: a) a genetically engineered cell that expresses on its cell surface at least one heterologous ligand-binding polypeptide; and b) a first copolymer drug composition, wherein the first copolymer drug composition comprises a ligand that specifically binds the heterologous ligand-binding polypeptide described in (a), such that the first copolymer drug composition is displayed on the surface of the genetically engineered cell, wherein the engineered cell further comprises a nucleic acid construct encoding a polypeptide, operably linked to a regulatory nucleic acid sequence that renders expression of the polypeptide modulated by the presence or absence of a first drug comprised by the first copolymer drug composition.

58. The composition of paragraph 57, wherein the genetically engineered cell further comprises a second copolymer drug composition.

59. The composition of paragraph 58, wherein the first drug comprised by the first copolymer drug composition is different from the second drug comprised by the second copolymer drug composition.

60. The composition of paragraph 59, wherein the second drug does not bind to or act on the regulatory nucleic acid sequence.

61. The composition of paragraph 60, wherein the second drug comprises a therapeutic agent that acts on a target cell (e.g., a cancer cell, a macrophage), a cellular microenvironment (e.g., tumor microenvironment), etc.

62. Use of a composition of any one of paragraphs 1-29 or 57-60 in the treatment of cancer or an autoimmune disease or disorder.

### EXAMPLES

### Example 1: Synthesis of a Representative CMP8 Drug Copolymer Composition

In this example the synthesis of a representative CMP8 drug copolymer is described.

POLY(tquat-co-CMP8SMA-co-FIMA): CMP8-SMA (100 mg, 1.3×10-4 mol), tquat (317 mg, 9.0×10⁻⁴ mol), FIMA (52 mg, 9.0×10⁻⁵ mol) and CTP (6.3 mg, 2.3×10⁻⁵ mol) were dissolved in 1.4 mL anhydrous DMSO. To this, 204 of freshly prepared ABCVA solution of 65 mg/mL concentration in DMSO (1.3 mg, 4.5×10⁻⁶ mol) was added. The reaction mixture was degassed by purging with nitrogen for 30 min. The reaction flask was sealed and heated at 70° C. for 18 h. The solution was cooled to room temperature and purified by precipitating in ether. The precipitate was washed with ether and then dried under high vacuum overnight. Yield=374 mg.

POLY(CB-co-CMP8SMA-co-FIMA): Tert-butyl protected polymer 300 mg from the previous step was treated with 6 mL trifluoroacetic acid at 4° C. After 5 minutes at 4° C., the reaction mixture was stirred at room temperature for 4.5 hours. Polymer solution was precipitated in ether, and the precipitate was washed with ether and dried under high vacuum. The polymer was further purified using PD-10 desalting columns and then lyophilized for 48 hours. Yield=147 mg.

Target DP: 50

| | | Mol % of | Wt % of | Wt % of |
|---|---|---|---|---|
| Monomer | MW | monomer | monomer | drug |
| tquat | 352 | 83 | 62 | |
| CMA8-SMA | 740 | 8 | 20 | 14.6 |
| FIMA | 574 | 9 | 18 | 11.3 |

### Synthesis of CMP8 Monomer

Mono-2-(methacryloyloxy)ethyl succinate 345 mg (1.5 mmol) and N,N,N′,N′-tetramethyl-O-(1H-benzotriazol-1-yl)uronium hexafluorophosphate (HBTU) 682 mg (1.8 mmol) in 300 mL CH2Cl2 was cooled to 0° C. To this solution, N,N-diisopropylethylamine 525 μL (3 mmol) was added, followed by N,N-dimethylpyridin-4-amine (DMAP) 30 mg (0.25 mmol). After 10 min at 0° C., the reaction mixture was stirred at room temperature for 30 min. CMP8 (660 mg, 1.25 mmol) was introduced and the reaction was continuously stirred at room temperature for 16 h. Solvent was evaporated under reduced pressure and the resulting oily crude residue was purified by silica gel column chromatography using 10% methanol in chloroform. Yield=740 mg (80%).

### Fluorescein Monomer

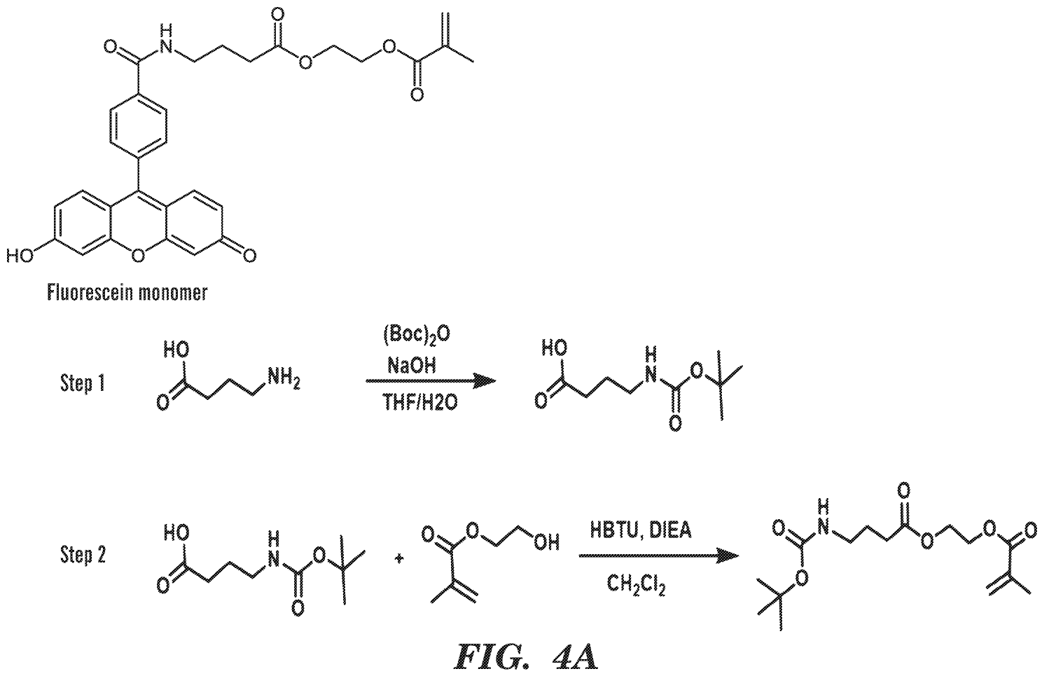

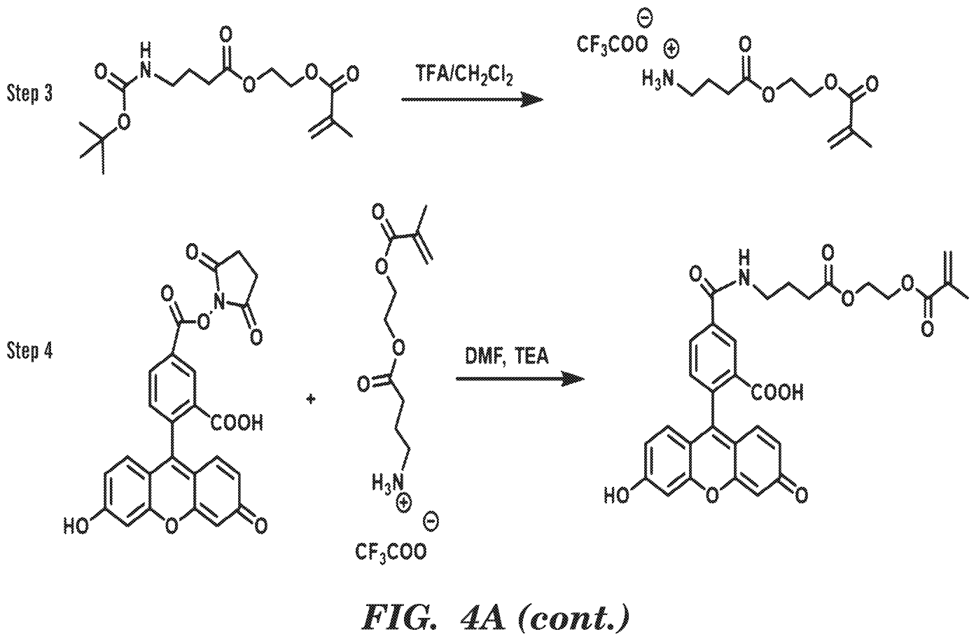

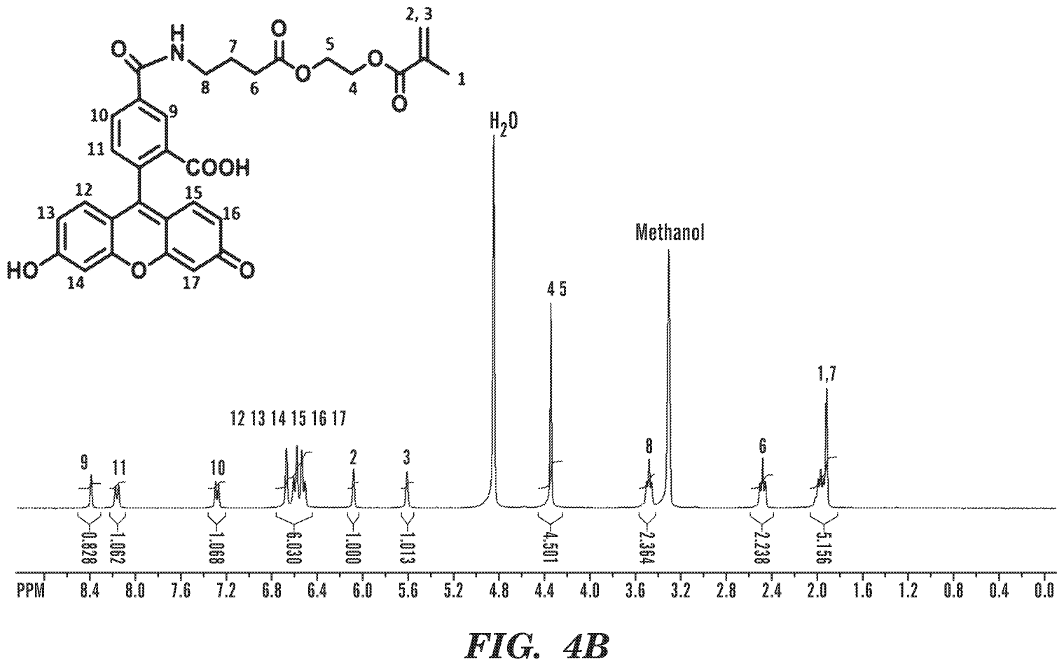

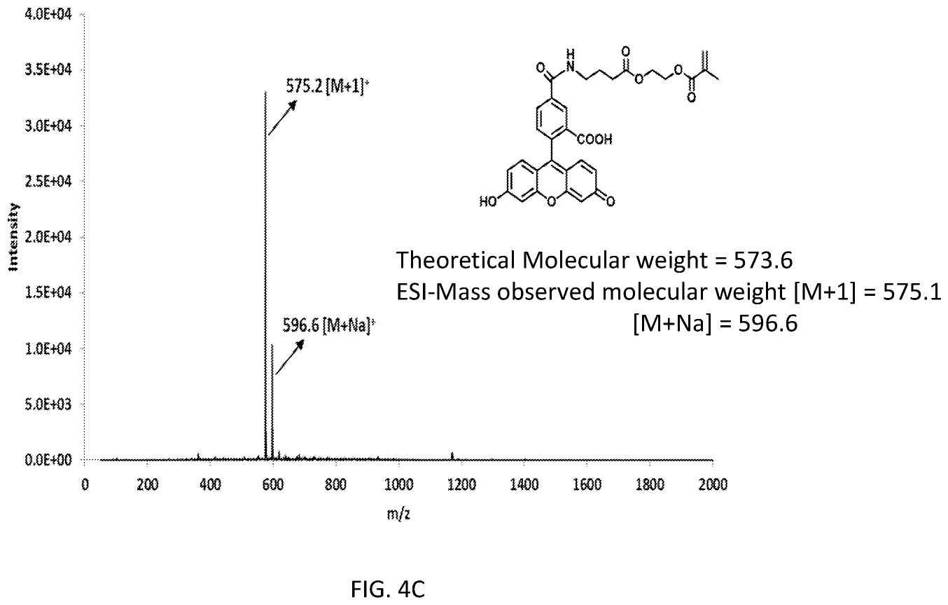

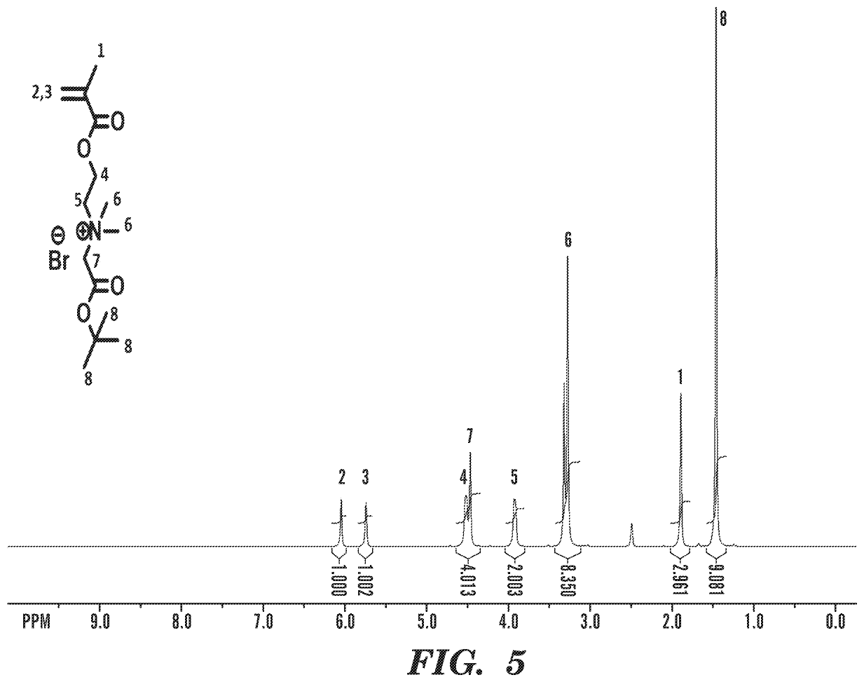

Methacryloyloxyethyl 4-aminobutanoate TFA salt 658 mg (2 mmol) in 10 mL DMF was treated with triethylamine 1.12 mL (8 mmol) under ice-cold condition. After 10 min, ice bath was removed and the mixture was stirred at room temperature for 20 min. 5-Carboxyfluorescein succinimidyl ester 800 mg (1.7 mmol) was added as solid and stirring was continued for 8 h protected from light. Solvent and volatiles were removed under reduced pressure and the residue was purified by silica gel chromatography with 8% methanol/chloroform as eluent. Yield=810 mg (83.07%). The synthesis of fluorescein monomer is shown in FIG. 4A. FIG. 4B shows ¹H-NMR spectrum data for the fluorescein monomer. FIG. 4C shows ESI-Mass spectrum data for the fluorescein monomer.

### Methacryloyloxyethyl 4-(tert-butoxycarbonylamino)butanoate

To 4-(tert-butoxycarbonylamino)butanoic acid 2.03 g (10 mmol) (for synthesis of 4-(ter-Butoxycarbonylamino)butanoic acid see e.g., Angew. Chem. Int. Ed. 2008, 47, 2700-270; data not shown) in 60 mL CH₂Cl₂ was added N,N-dimethylpyridin-4-amine 1.22 g (10 mmol), N—N′-dicyclohexylcarbodimide 2.27 g (11 mmol) and 2-hydroxylethyl methacrylate 1.3 g (10 mmol). The reaction mixture was stirred at room temperature for 5 h. The byproduct dicyclohexylurea was filtered off, and the filtrate was concentrated under reduced pressure. The crude product was purified by silica gel chromatography using 4.5% methanol/chloroform as eluent. Yield: 3.23 g (contains 2-3% DCU).

### Methacryloyloxyethyl 4-aminobutanoate TFA Salt

Methacryloyloxyethyl 4-(tert-butoxycarbonylamino)butanoate 3 g (9.5 mmol) in 20 mL 40% TFA/dichloromethane was stirred at room temperature for 3.5 h. Solvent was removed and the residue was treated with 30% ether/hexane (150 mL). This was centrifuged after keeping at −20° C. for 2 h. Oily product at the bottom was collected by carefully decanting the supernatant. This process was repeated two more times. Product was dried by purging with air. Yield=Quantitative.

### Example 2: Synthesis of Rhodamine Ligand Monomers

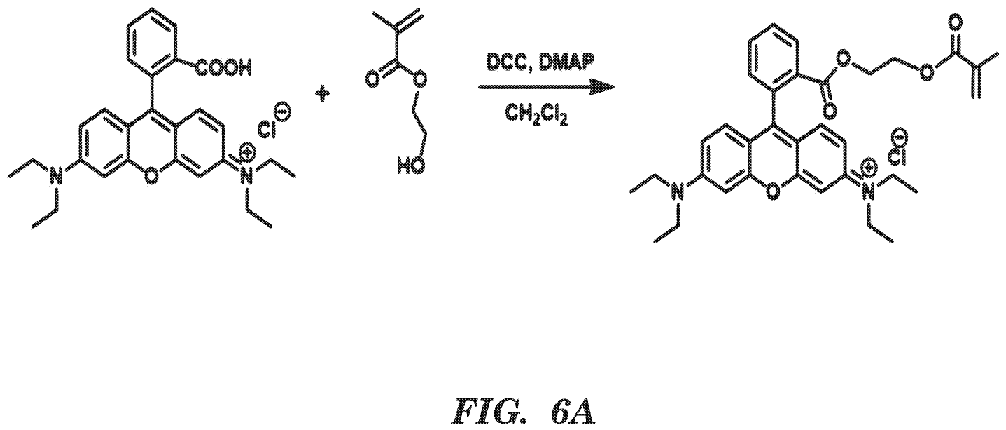

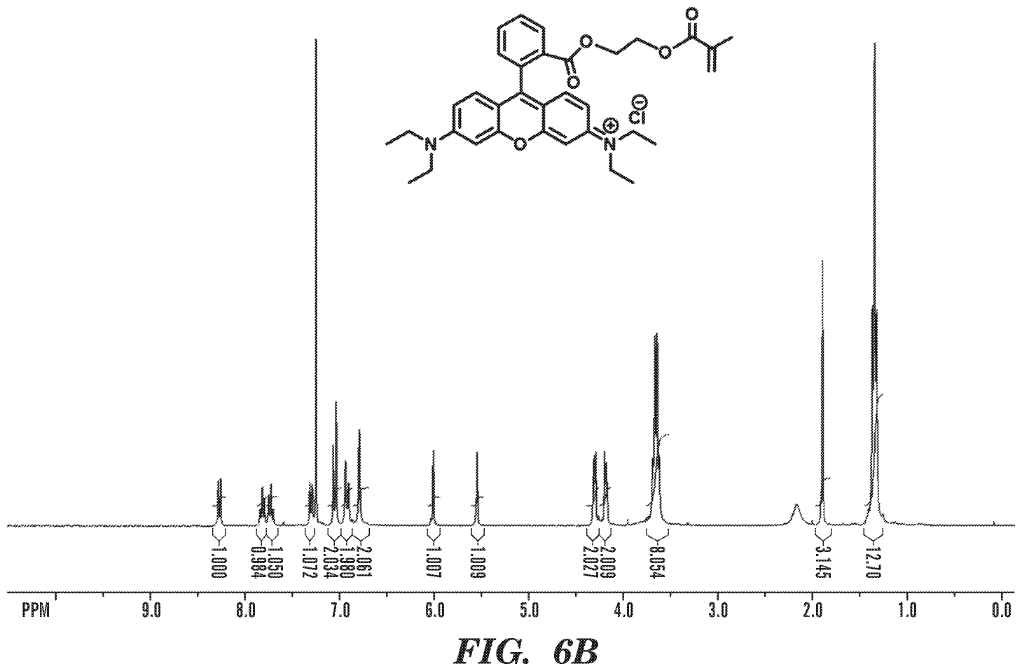

In this example, the synthesis of ligand monomers, in addition to the fluorescein monomer described in Example 1 and useful in embodiments of the technology described herein is described. Rhodamine-HEMA monomer Synthesis is illustrated schematically in FIG. 6A.

To a solution of rhodamine B 5.27 g (11 mmol), N,N′-dicyclohexylcarbodimide 2.88 g (14 mmol) and 4-dimethylaminopyridine 134 mg (1.1 mmol) in 75 mL CH₂Cl₂ was added 2-hydroxyethyl methacrylate 1.82 g (14 mmol) at 0 degrees C. After 30 min, the ice bath was removed and the reaction mixture was stirred at room temperature for 16 h. After filtering off the byproduct dicyclohexylurea, the solvent was evaporated under reduced pressure. The residue was redissolved in 30 mL acetonitrile and the insoluble materials were filtered off. The crude product obtained after evaporating acetonitrile was purified by flash column chromatography using 6% methanol in chloroform. Yield: 5.89 g (91%). An exemplary ¹H-NMR spectrum of Rhodamine monomer is shown in FIG. 6B.

### Example 3: Synthesis of Additional Ligand Monomers

### PI103-SMA Monomer

To a solution of mono-2-(methacryloyloxy)ethyl succinate (SMA) 759 mg (3.3 mmol), N-(3-dimethylaminopropyl)-N′-ethylcarbodimide hydrochloride (EDCI.HCl) 1.26 g (6.6 mmol) and N,N-dimethylpyridin-4-amine (DMAP) 403 mg (3.3 mmol) in 200 mL anhydrous CH2C12 was added PI103 766 mg (2.2 mmol) as solid at 0° C. After 5 minutes at 0° C., the reaction mixture was stirred at room temperature for 3 h. After evaporation of the solvent under reduced pressure, the resulting crude product was purified by silica gel column chromatography using 22% tetrahydrofuran in chloroform as column eluent. Column purified product was dissolved in 8 mL column eluent, precipitated in 20% ether/hexane and kept at −20° C. overnight to complete the precipitation. The precipitate was filtered, washed with cold 20% ether/hexane and dried under high vacuum. Yield=962 mg (78.02%).

### 4-Hydroxytamoxifen-SMA Monomer

To a solution of mono-2-(methacryloyloxy)ethyl succinate (SMA) 414 mg (1.8 mmol) in 50 mL anhydrous CH2Cl2 at 0° C., was added N-(3-dimethylaminopropyl)-N′-ethylcarbodimide hydrochloride (EDCI.HCl) 575 mg (3.0 mmol) and N,N-dimethylpyridin-4-amine (DMAP) 220 mg (1.8 mmol). After stirring the reaction mixture at 0° C. for 15 minutes and at room temperature for 15 minutes, 4-hydroxytamoxifen 465 mg (1.2 mmol) was added as solid and the stirring was continued for 16 h. After evaporation of the solvent under reduced pressure, the resulting crude product was purified by silica gel column chromatography using methanol/dichloromethane/triethylamine (10/89.5/0.5) as column eluent. Yield=691 mg (96.02%) (data not shown).

### 4-(Hydroxymethyl)phenyl (2-(methacryloyloxy)ethyl) succinate

N-(3-Dimethylaminopropyl)-N′-ethylcarbodiimide hydrochloride (20 g, 104 mmol) was added in portions to a stirred, chilled (0-5° C.) solution of mono-2-(methacryloyloxy)ethyl succinate (23.02 g, 100 mmol), 2-mercaptothiazoline (11.90 g, 100 mmol) and 4-dimethylaminopyridine (12.20 g, 100 mmol) in dichloromethane (500 mL). The resulting solution was stirred at 0-5° C. for 30 min, then allowed to slowly warm to room temperature and stirred overnight. The dichloromethane was removed under reduced pressure to yield a highly viscous oil. This was extracted with 200 mL of diethyl ether with mechanical stirring for 30 min, then the ether decanted off. This process was repeated twice more, then twice more with two 100 mL aliquots of ether. A few crystals of di-tert-butyl-4-methylphenol were added then the combined ether extracts were concentrated under reduced pressure to a tan gum which was used in the next step without further purification.

A suspension of 4-hydroxybenzyl alcohol (18.6 g, 150 mmol) in dichloromethane was added portion-wise to a stirred, chilled (0-5° C.) solution of the activated ester from the previous step and 4− dimethylaminopyridine (12.20 g, 100 mmol) in dichloromethane (700 mL). The reaction mixture was stirred at 0-5° C. for 20 min then allowed to warm slowly to room temperature and stirred overnight. It was then washed with 1M hydrochloric acid (3×200 mL), and warm water (40° C.) (3×200 mL). The organic phase was dried (MgSO₄), filtered and concentrated under reduced pressure to a brown semisolid. The crude product was partially purified by flash vacuum chromatography with 1:3 then 1:1 ethyl acetate:petroleum spirit. The chromatographed material was triturated repeatedly with ethyl acetate, the extract concentrated, then triturated repeatedly with diethyl ether and the extract concentrated to give 4-(hydroxymethyl)phenyl (2-(methacryloyloxy)ethyl) succinate containing 19 mol % of 2-mercaptothiazoline (20.982 g, 57% over 2 steps). ¹H NMR (400 MHz, CDCl₃) 7.34 (d, J=8.3 Hz, 2H), 7.05 (d, J=8.3 Hz, 2H), 6.10 (s, 1H), 5.55 (quintet, J=1.5 Hz, 1H), 4.65 (s, 2H), 4.38-4.30 (m, 4H), 2.86 (m, 2H), 2.74 (m, 2H), 1.91 (s, 3H) [mercaptothiazoline peaks 3.92 (dt, J=7, 3 Hz, 2H), 3.52 (dt, J=7, 3 Hz, 2H)] (data not shown). This material was used as is in further procedures.

### 4-((((2-(Ethoxymethyl)-1-(2-hydroxy-2-methylpropyl)-1H-imidazo[4,5-c]quinolin-4-yl)carbamoyl)oxy)methyl)phenyl (2-(methacryloyloxy)ethyl) succinate

To a mixture of resiquimod (3.94 g, 12.5 mmol) in CH₂Cl₂ (100 mL) and MeCN (200 mL) was added 1,1′-carbonyldiimidazole (2.7 g of ca 90% purity, 15 mmol). After two hours a further portion of 1,1′-carbonyldiimidazole (470 mg of ca 90% purity, 2.6 mmol) was added and the mixture stirred for a further two hours. To this mixture was added 4-(hydroxymethyl)phenyl (2-(methacryloyloxy)ethyl) succinate (7.1 g of ca 90% purity, 19 mmol) and the mixture stirred 16 h. The mixture was diluted with CH₂Cl₂ and washed with H₂O and then brine, dried (MgSO₄), filtered and concentrated. Silica chromatography MeOH/CH₂Cl₂ (1:99 then 2.5:97.5) afforded 4.6 g of a mixture containing 44(2− (methacryloyloxy)ethyl)succinyloxy)benzyloxycarbonyl-Resiquimod (3.5 g, 41%) and ethyl acetate (1.1 g). ¹H NMR (400 MHz, CDCl₃) δ 1.17 (t, J=7.0 Hz, 3H), 1.27, br s, 6H), 1.89, (s, 3H), 2.70-2.75 (m, 2H), 2.82-2.89 (m, 2H), 3.57 (q, J=7.0 Hz, 2H), 4.28-4.36 (m, 4H), 4.71 (br s, 2H), 4.83 (br s, 2H), 5.53 (s, 1H), 6.07 (s, 1H), 7.02-7.08 (m, 2H), 7.40-7.48 (m, 3H), 7.50-7.56 (m, 1H), 8.10 (ddd, J=1.1, 8.4, 16.1 Hz, 1H) (data not shown).

### 7-Cyano-7-methyl-4-oxo-9-thioxo-8,10-dithia-3-azadodecanyl-Rhodamine B

Trifluoroacetic acid (6 mL) was added to a cooled (0-5° C.) mixture of t-butoxycarbonylaminoethyl-Rhodamine B (780 mg, 1.25 mmol) and dichloromethane (30 mL) was cooled (0° C.). After 1 h, the mixture was diluted with dichloromethane and washed sequentially with water, saturated aqueous NaHCO₃and then brine, dried (MgSO₄), filtered and concentrated to ca 60 mL. To this mixture was added 2,5-dioxopyrrolidin-1-yl 4-cyano-4-(((ethylthio)carbonothioyl)thio)pentanoate (600 mg, 1.7 mmol) and ⁱPr₂EtN (600 μL). After 1 h, the mixture was diluted with CH₂Cl₂ and washed sequentially with H₂O, saturated aqueous NaHCO₃and then brine, dried (MgSO₄), filtered and concentrated. Purification by silica chromatography MeOH/CH₂Cl₂ (2.5:97.5 then 5:95 then 8:92) afforded 7-cyano-7-methyl-4-oxo-9-thioxo-8,10-dithia-3-azadodecanyl-Rhodamine B (560 mg, 58%). ¹H NMR (400 MHz, CDCl₃) δ 1.28-1.34 (m, 15H), 1.89 (s, 3H), 2.36-2.46 (m, 1H), 2.50-2.72 (m, 3H), 3.26-3.38 (m, 4H), 3.50-3.67 (m, 8H), 4.17 (t, J=5.6 Hz, 2H), 6.71-6.74 (m, 2H), 6.92 (dd, J=2.4, 8.5 Hz, 2H), 7.12-7.19 (m, 3H), 7.67-7.79 (m, 2H), 8.53 (dd, J=1.0, 7.8 Hz, 1H), 9.15 (br s, 1H).

### 5-Carboxyfluorescein 4-(2-(methacryloyloxy)ethoxy)-4-oxobutylamide

### 2-(Methacryloyloxy)ethyl 4-((tert-butoxycarbonyl)amino)butanoate

N-(3-Dimethylaminopropyl)-N-ethylcarbodiimide hydrochloride (4.89 g, 25.5 mmol), 4-dimethylaminopyridine (2.82 g, 23 mmol) and 2-hydroxyethyl methacrylate (2.80 mL, 23 mmol) were added to a stirred solution of t-butoxycarbonylaminobutyric acid (4.71 g, 23 mmol) in dichloromethane (140 mL). The solution was stirred overnight then washed with water (3×100 mL), dried (MgSO₄), filtered through silica and concentrated under reduced pressure to a near colourless oil. This was taken up in dichloromethane and subjected to flash vacuum chromatography (dichloromethane elution) to give 2− (Methacryloyloxy)ethyl 4-((tert-butoxycarbonyl)amino)-butanoate (3.89 g, 48% yield) as a colourless oil. NMR (400 MHz, CDCl₃) 6.11 (t, J=1.2 Hz, 1H), 5.58 (quintet, J=1.5 Hz, 1H), 4.64 (bs, <1H), 4.36-4.29 (m, 4H), 3.20-3.10 (m, 2H), 2.36 (t, J=7.4 Hz, 2H), 1.80 (quintet, J=7.3 Hz, 2H), 1.42 (s, 9H) (data not shown).

### 2-(Methacryloyloxy)ethyl 4-aminobutanoate trifluoroacetic acid salt

2-(Methacryloyloxy)ethyl 4-((tert-butoxycarbonyl)amino)butanoate (3.89 g, 12.3 mmol) in a 40% solution of trifluoroacetic acid in dichloromethane (25 mL) was stirred at room temperature for 3.5 h. The solvent was removed and the residue stirred for 1 h with 33% ether in petroleum spirit, then cooled to −14° C. for 30 min before the supernatant was decanted off. This process was repeated twice more, then the residue concentrated under a stream of air to give 2-(methacryloyloxy)ethyl 4-aminobutanoate trifluoroacetic acid salt (3.37 g, 83%) as a pale tan gum. NMR (400 MHz, CDCl₃) 8.05 (bs, >1H), 6.07 (t, J=1.0 Hz, 1H), 5.56 (quintet, J=1.5 Hz, 1H), 4.33-4.25 (m, 4H), 3.21 (bs, <1H), 3.00 (t, J=7.2 Hz, 2H), 2.44 (t, J=7.0 Hz, 2H), 1.95 (quintet, J=7.2 Hz, 2H), 1.89 (dd, J=1.5, 1.0 Hz) (data not shown).

### 5-Carboxyfluorescein 4-(2-(methacryloyloxy)ethoxy)-4-oxobutylamide

Triethylamine (1.12 mL, 8 mmol) was added to a stirred, chilled (0-5° C.) solution of 2− (methacryl-oyloxy)ethyl 4-aminobutanoate trifluoroacetic acid salt (658 mg, 2 mmol) in dimethylformamide (10 mL) and the resulting mixture stirred for 10 min. The ice bath was then removed and the mixture stirred at room temperature for a further 20 min, then 5-carboxyfluorescein succinimidyl ester (800 mg, 1.7 mmol) added and stirring continued for 8h with protection from the light. The volatiles were removed under reduced pressure and the residue purified by chromatography on silica (eluent 8% methanol in chloroform) to give 5-Carboxyfluorescein 4-(2-(methacryloyloxy)ethoxy)-4-oxobutylamide (810 mg, 83% yield). 1H NMR (400 MHz, CD₃OD) 8.77 (t, J=5.7 Hz, <1H), 8.16 (dd, J=8.1, 1.6 Hz, 1H), 7.26 (dd, J=8.1, 0.6 Hz, 1H), 6.65 (d, J=2.3 Hz, 2H), 6.56 (d, J=8.7 Hz, 2H), 6.50 (dd, J=8.7, 2.3 Hz, 2H), 6.07-6.05 (m, 1H), 5.59 (quintet, J=1.5 Hz, 1H), 4.32 (s, 4H), 3.48-3.42 (m, 2H), 2.44 (t, J=7.3 Hz, 2H), 1.93 (quintet, J=7.3 Hz, 2H), 1.88 (dd, J=1.0, 1.5 Hz, 3H) (data not shown).

### Galunisertib Monomer

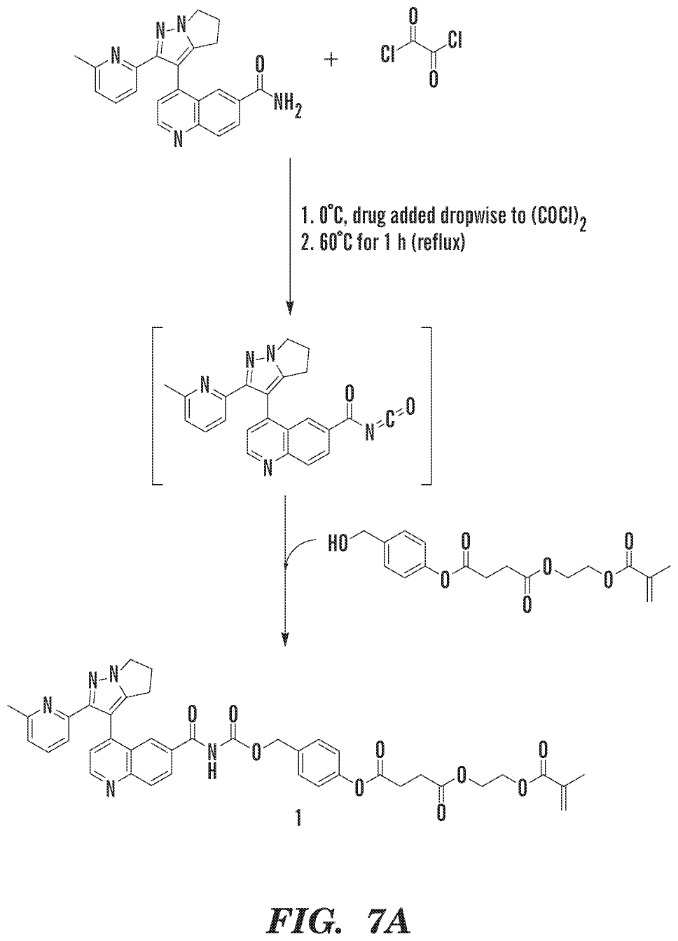

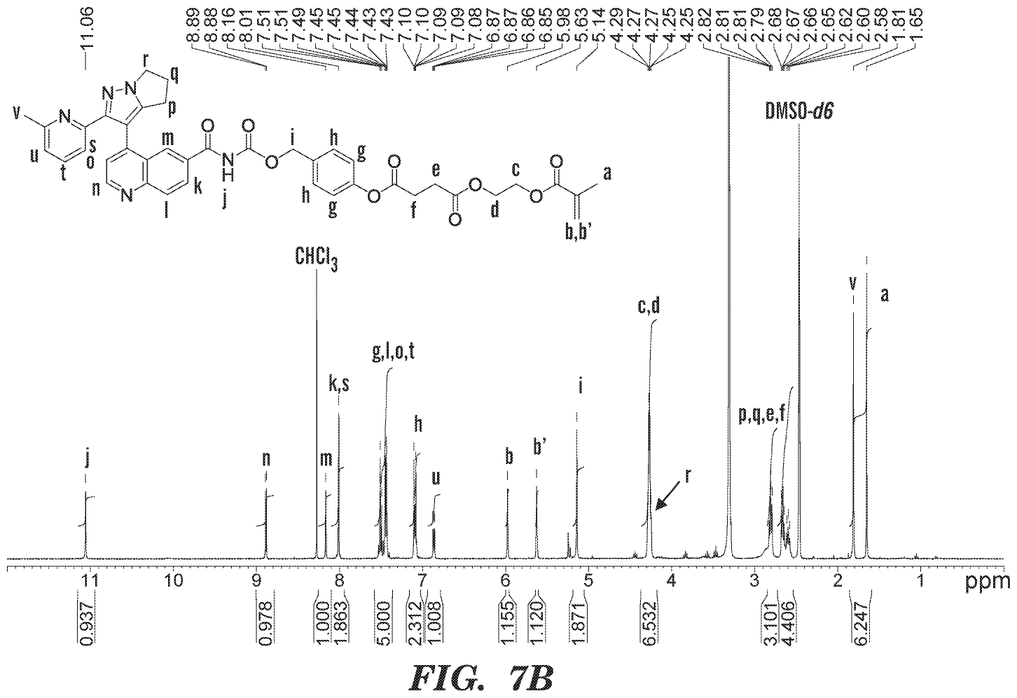

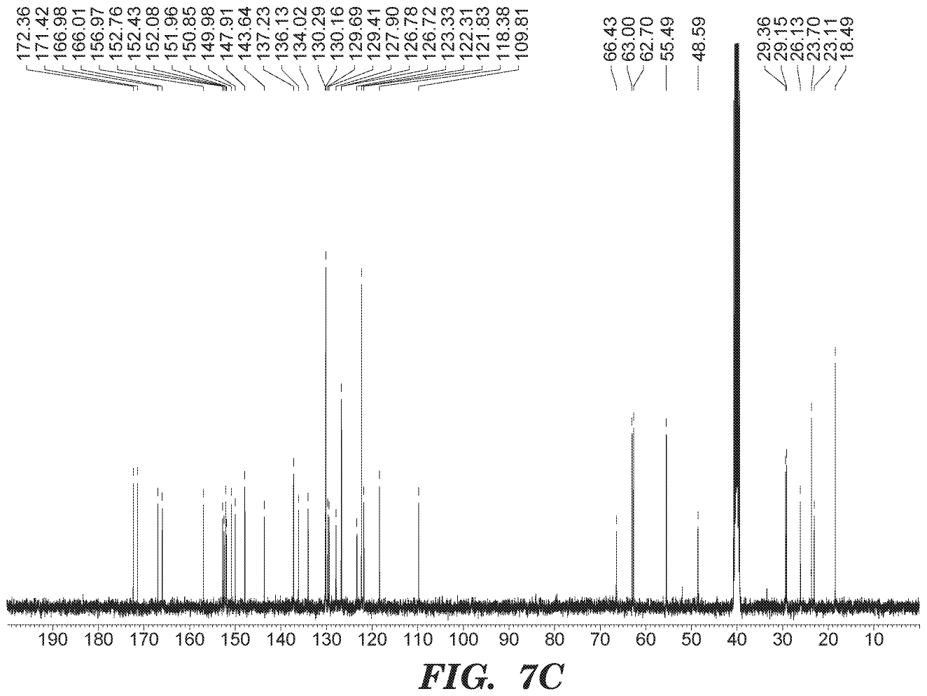

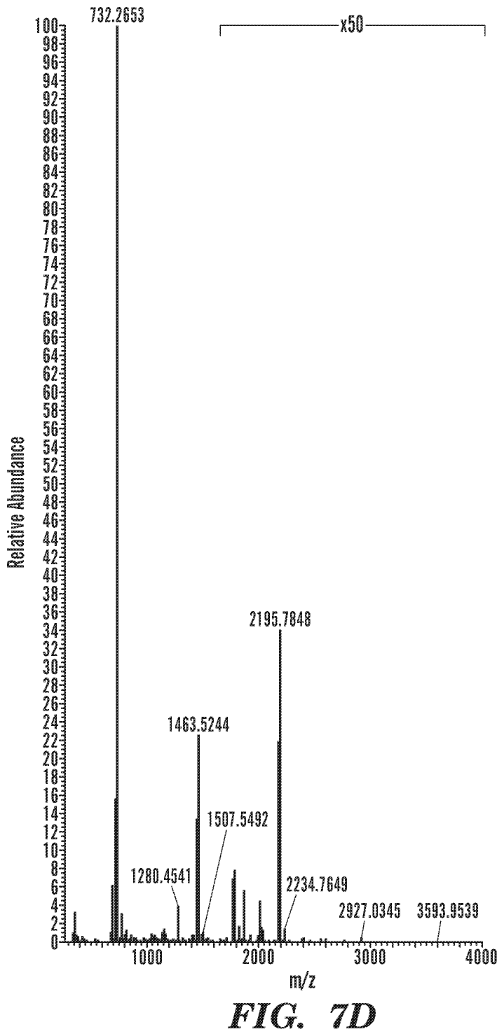

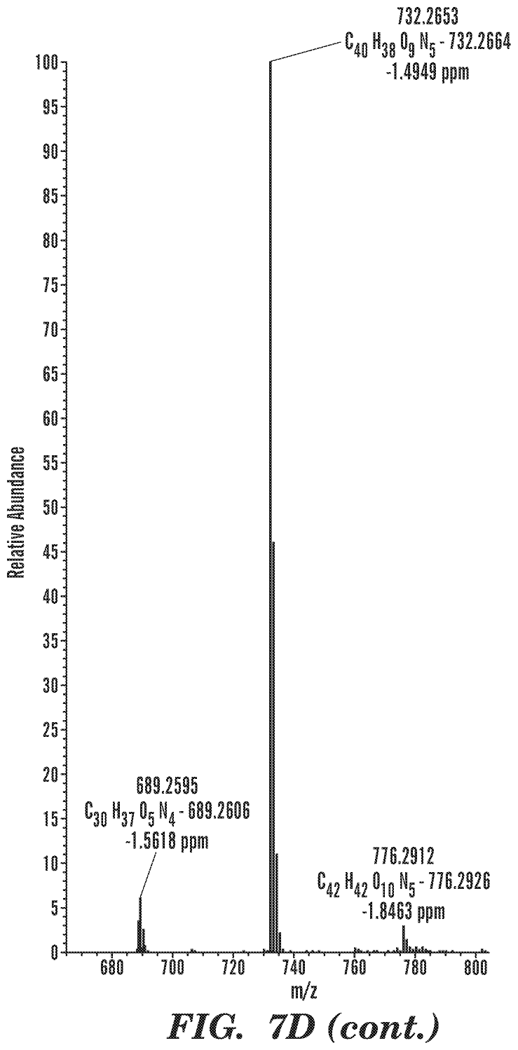

Galunisertib (1.676 g, 4.53 mmol) was dissolved in CHCl3 (42.8 mL) and added dropwise to oxalyl chloride (540.5 uL, 6.35 mmol, 1.4 equiv) in MeCN (12.5 mL) that was placed in an ice-bath. Caution—this reaction is highly exothermic. The solution is then stirred on ice for a further 45 min before refluxing at 60° C. for 2.5 hr. Next, the reaction mixture is once again placed in an ice-bath. The benzylalcohol methacrylate monomer (2.135 g, 6.35 mmol, 1.4 equiv) in CHCl3 (2.0 mL) is then added dropwise to the reaction mixture containing galunisertib-carboxyisocyanate. After addition of the benzylalcohol methacrylate monomer is complete the reaction mixture was allowed to stir for 2 hours at room temperature. The reaction is then diluted with CHCl₃ (60 mL), quenched with a saturated solution of KHCO₃ (100 mL). The organic layer is collected, washed with water (100 mL), with brine (100 mL), dried over NaSO₄ and filtered. Rotary evaporation of the filtrate yielded a viscous brown liquid. Further evacuation of residual solvent yielded a brown solid. Rf=0.4. Dry silica (0.020-0.045 micron) flash chromatography with CHCl₃→CHCl₃/MeOH (92/8 v/v) as eluent. Co-elution of unreacted benzylalcohol methacrylate can be removed by isolation of product from diethyl ether/pentane (1/1 v/v). Centrifugation (5000 rpm) resulted in efficient recovery of precipitated product (90%). NMR spectra and mass spectrometry was performed to confirm synthesis of the monomer (FIGS. 7B-7D).

### Example 4: Synthesis of Poly(DMA-c0-PI103-SMA)

PI103-SMA (90 mg, 1.6×10⁻⁴ mol), DMA (211 mg, 2.1×10⁻³ mol) and ECT (6.0 mg, 2.3×10-5 mol) were dissolved in 750 μL 1,4-dioxane. To this, 10 μL of freshly prepared ABCVA solution of 64 mg/mL concentration in 1,4-dioxane (0.64 mg, 2.3×10⁻⁶ mol) was added. The reaction mixture was degassed by purging with nitrogen for 30 min. The reaction flask was sealed and heated at 70° C. for 6 h. The polymer solution was diluted with tetrahydrofuran (4 mL) and precipitated in ether. The precipitate was washed with ether and then dried under high vacuum overnight. The polymer was further purified using PD-10 desalting columns and then lyophilized for 48 hours. Yield=210 mg (data not shown).

Target DP: 100

| | | Mol % of | Wt % of | Wt % of |
|---|---|---|---|---|
| Monomer | MW | monomer | monomer | drug |
| DMA | 99 | 92 | 67 | |
| PI103-SMA | 560.6 | 8 | 33 | 20.5 |

An exemplary ¹H-NMR spectrum of POLY(DMA-co-PI103-SMA) is shown in FIG. 14B. GPC chromatogram of POLY(DMA-co-PI103-SMA) is shown in FIG. 14C.

### Example 5: Synthesis of a Representative Combination Drug Copolymer

In this example the synthesis of a representative combination drug copolymer (Resiquimod and Galunisertib) is described.

### PEGMA/ResiquimodMA/GalunisertibMA/FluoresceinMA/Rhodamine Polymer MW24K Synthesis

After polymerisation and purification, DP: 29, MW: 23963 (including RAFT agent)

| Monomer | MW | # of units/chain | Mol % | Wt % |
|---|---|---|---|---|
| PEGMA | 950 | 14 | 47 | 55.5 |
| ResiquimodMA | 676.7 | 6.4 | 21.5 | 17.8 |
| GalunisertibMA | 732.3 | 5.4 | 18.1 | 16.5 |
| FluoresceinMA | 559.5 | 3 | 10 | 7 |
| RhodamineCTA | 767.5 | 1 | 3.4 | 3.2 |

Poly(ethylene glycol methacrylate), MW: 950 g/mol, (PEGMA 950) (285 mg, 0.3 mmol), ResiquimodMA (67.7 mg, 0.1 mmol), GalunisertibMA (73.2 mg, 0.1 mmol), FluoresceinMA (27.9 mg, 0.05 mmol), RhodamineCTA (7.7 mg, 0.01 mmol) and 2,2′-Azobis(4-methoxy-2,4-dimethylvaleronitrile) (0.77 mg, 0.0025 mmol) were dissolved in DMF (0.6 ml, 7.8 mmol). The initial monomer to CTA molar ratio ([M]₀:[CTA]₀) was 55:1 and the initial CTA to initiator ratio ([CTA]₀:[I]₀) was 4:1. The molar ratio of PEGMA to ResiquimodMA to GalunisertibMA to FluoresceinMA monomers was 30:10:10:5. The solution was purged with nitrogen for 30 min and was then heated at 30° C. for 20 h. The product was purified via dialysis against acetone/water (9/1 v/v) (72 h). Acetone was removed by rotavapor and water was removed by freeze drying. Successful monomer incorporation of PEGMA 950, ResiquimodMA, GalunisertibMA and FluoresceinMA was determined by ¹H NMR (MeOD), the PEGMA 950 peak is seen at δ3.56 (data not shown). ResiquimodMA peak can be seen at δ8.31 (data not shown). GalunisertibMA peak can be seen at δ8.79 (data not shown). FluoresceinMA peaks can be seen at δ6.51, δ6.65 (data not shown).

### PEGMA/ResiquimodMA/GalunisertibMA/FluoresceinMA/Rhodamine Polymer MW48K Synthesis

After polymerisation and purification, DP: 60, MW: 48477 (including RAFT agent)

| Monomer | MW | # of units/chain | Mol % | Wt % |
|---|---|---|---|---|
| PEGMA | 950 | 26 | 42.5 | 51 |
| ResiquimodMA | 676.7 | 14.2 | 23.2 | 19.8 |
| GalunisertibMA | 732.3 | 12.8 | 20.9 | 19.3 |
| FluoresceinMA | 559.5 | 7.2 | 11.8 | 8.3 |
| RhodamineCTA | 767.5 | 1 | 1.6 | 1.6 |

Poly(ethylene glycol methacrylate), MW: 950 g/mol, (PEGMA 950) (285 mg, 0.3 mmol), ResiquimodMA (101.5 mg, 0.15 mmol), GalunisertibMA (110.2 mg, 0.15 mmol), FluoresceinMA (44.7 mg, 0.08 mmol), RhodamineCTA (7.7 mg, 0.01 mmol) and 2,2′-Azobis(4-methoxy-2,4− dimethylvaleronitrile) (0.77 mg, 0.0025 mmol) were dissolved in DMF (0.6 ml, 7.8 mmol). The initial monomer to CTA molar ratio ([M]₀:[CTA]₀) was 68:1 and the initial CTA to initiator ratio ([CTA]₀:[I]₀) was 4:1. The molar ratio of PEGMA to ResiquimodMA to GalunisertibMA to FluoresceinMA monomers was 30:15:15:8. The solution was purged with nitrogen for 30 min and was then heated at 30° C. for 20 h. The product was purified via dialysis against acetone/water (9/1 v/v) (72 h). Acetone was removed by rotavapor and water was removed by freeze drying. Successful monomer incorporation of PEGMA 950, ResiquimodMA, GalunisertibMA and FluoresceinMA was determined by ¹H NMR (MeOD) (data not shown), the PEGMA 950 peak is seen at δ3.55 (data not shown). ResiquimodMA peak can be seen at δ8.31 (data not shown). GalunisertibMA peak can be seen at δ8.78 (data not shown). FluoresceinMA peaks can be seen at δ6.50, δ6.64 (data not shown).

1. A composition comprising:
a) a genetically engineered cell that expresses on its cell surface at least one heterologous ligand-binding polypeptide; and
b) at least one copolymer drug composition, wherein each of the at least one copolymer drug composition comprises a ligand that specifically binds the heterologous ligand-binding polypeptide described in (a), such that the at least one copolymer drug composition is displayed on the surface of the genetically engineered cell,
wherein the engineered cell further comprises a nucleic acid construct encoding a polypeptide, operably linked to a regulatory nucleic acid sequence that renders expression of the polypeptide modulated by the presence or absence of a drug comprised by the copolymer drug composition.

2. The composition of claim 1, wherein the at least one heterologous ligand-binding polypeptide comprises an antigen binding domain of an antibody that binds the ligand comprised by a copolymer drug composition.

3. The composition of claim 1, wherein the genetically-engineered cell further expresses a heterologous receptor that binds a cell-surface ligand on a target cell.

4. The composition of claim 1, wherein the genetically engineered cell is a T cell, a macrophage or a stem cell.

5.-10. (canceled)

11. The composition of claim 1, wherein the drug comprised by the copolymer drug composition is required for the expression of the polypeptide.

12.-15. (canceled)

16. The composition of claim 1, wherein the expression of the polypeptide is repressed by the drug comprised by the copolymer drug composition.

17. The composition of claim 16, wherein the polypeptide promotes the death of the genetically engineered cell.

18. The composition of claim 1, wherein the polypeptide modulates an activity of a target cell.

19.-22. (canceled)

23. The composition of claim 1, wherein the polypeptide comprises an antigen-binding domain of an antibody.

24.-29. (canceled)

30. A method of administering a polypeptide of interest to an individual in need thereof, the method comprising administering to the individual a composition of claim 1, wherein a drug comprised by the copolymer drug composition is released from the copolymer after the composition is administered, and wherein that drug induces expression of the polypeptide wherein the expression of the polypeptide continues only while that drug is present.

31. (canceled)

32. The method of claim 30, wherein the genetically engineered cell is contacted with the at least one copolymer drug composition before the genetically engineered cell is administered.

33. The method of claim 30, wherein the genetically-engineered cell further expresses a heterologous receptor that binds a cell-surface ligand on a target cell.

34. The method of claim 33, wherein the target cell is a tumor cell and wherein the polypeptide of interest promotes tumor cell death.

35.-45. (canceled)

46. The method of claim 30, wherein the polypeptide of interest modulates an activity of a target cell.

47.-52. (canceled)

53. A method of limiting the duration of a cell-mediated therapy in an individual in need thereof, the method comprising administering to the individual a composition comprising:
a) a genetically engineered cell that expresses on its cell surface at least one heterologous ligand-binding polypeptide; and
b) at least one copolymer drug composition, wherein each of the at least one copolymer drug composition comprises a ligand that specifically binds a heterologous ligand-binding polypeptide described in (a), such that the at least one copolymer drug composition is displayed on the surface of the genetically engineered cell,
wherein the engineered cell further comprises a nucleic acid construct encoding a first polypeptide of interest, operably linked to a regulatory nucleic acid sequence that renders expression of the first polypeptide of interest sensitive to the presence or absence of a drug comprised by a copolymer drug composition described in (b),
wherein the engineered cell further comprises a nucleic acid construct encoding a second polypeptide of interest,
wherein a drug comprised by a copolymer drug composition as described in (b) is released from the copolymer after the composition is administered, wherein a drug released from a copolymer composition represses the expression of the first polypeptide of interest,
wherein the second polypeptide of interest is a therapeutic polypeptide, and
wherein the first polypeptide of interest promotes the death of the genetically engineered cell.

54. The method of claim 53, wherein the first polypeptide comprises a toxin polypeptide.

55. (canceled)

56. The method of claim 53, wherein the drug that represses expression of the first polypeptide is selected from the group consisting of tetracycline and doxycycline.

57. A composition comprising: a) a genetically engineered cell that expresses on its cell surface at least one heterologous ligand-binding polypeptide; and b) a first copolymer drug composition, wherein the first copolymer drug composition comprises a ligand that specifically binds the heterologous ligand-binding polypeptide described in (a), such that the first copolymer drug composition is displayed on the surface of the genetically engineered cell, wherein the engineered cell further comprises a nucleic acid construct encoding a polypeptide, operably linked to a regulatory nucleic acid sequence that renders expression of the polypeptide modulated by the presence or absence of a first drug comprised by the first copolymer drug composition.

58. The composition of claim 57, wherein the genetically engineered cell further comprises a second copolymer drug composition.

59. The composition of claim 58, wherein the first drug comprised by the first copolymer drug composition is different from the second drug comprised by the second copolymer drug composition.

60. The composition of claim 59, wherein the second drug does not bind to or act on the regulatory nucleic acid sequence and/or comprises a therapeutic agent that acts on a target cell or cellular microenvironment.

61.-62. (canceled)


SEQUENCE LISTING (13 sequences)

SEQ ID NO: 1 - protein, 15 aa, Artificial Sequence
  Description of Artificial Sequence Synthetic peptide
GLNDIFEAQKIEWHE

SEQ ID NO: 2 - protein, 6 aa, Artificial Sequence
  Description of Artificial Sequence Synthetic 6xHis tag
HHHHHH

SEQ ID NO: 3 - protein, 10 aa, Artificial Sequence
  Description of Artificial Sequence Synthetic peptide
EQKLISEEDL

SEQ ID NO: 4 - protein, 9 aa, Artificial Sequence
  Description of Artificial Sequence Synthetic peptide
YPYDVPDYA

SEQ ID NO: 5 - protein, 8 aa, Artificial Sequence
  Description of Artificial Sequence Synthetic peptide
DYKDDDDK

SEQ ID NO: 6 - protein, 6 aa, Artificial Sequence
  Description of Artificial Sequence Synthetic peptide
LVPRGS

SEQ ID NO: 7 - protein, 14 aa, Artificial Sequence
  Description of Artificial Sequence Synthetic peptide
GKPIPNPLLGLDST

SEQ ID NO: 8 - protein, 10 aa, Artificial Sequence
  Description of Artificial Sequence Synthetic peptide
PRPSNKRLQQ

SEQ ID NO: 9 - protein, 12 aa, Artificial Sequence
  Description of Artificial Sequence Synthetic peptide
EDQVDPRLIDGK

SEQ ID NO: 10 - protein, 8 aa, Artificial Sequence
  Description of Artificial Sequence Synthetic peptide
PKKKRKVG

SEQ ID NO: 11 - protein, 11 aa, Artificial Sequence
  Description of Artificial Sequence Synthetic peptide
YTDIEMNRLGK

SEQ ID NO: 12 - protein, 4 aa, Artificial Sequence
  Description of Artificial Sequence Synthetic peptide
IDGR

SEQ ID NO: 13 - protein, 11 aa, Artificial Sequence
  Description of Artificial Sequence Synthetic peptide
MASMTGGQQMG